(19) United States
(12) Patent Application Publication
(10) Pub. No.: US 2024/0384296 A1
(43) Pub. Date: Nov. 21, 2024

(54) RECOMBINANT HCMV VECTORS AND USES THEREOF

(71) Applicant: Vir Biotechnology, Inc., San Francisco, CA (US)

(72) Inventors: Ann M. Arvin, San Francisco, CA (US); Janet L. Douglas, San Francisco, CA (US); Emily Marshall, San Francisco, CA (US); Herbert W. Virgin, San Francisco, CA (US)

(21) Appl. No.: 18/687,050

(22) PCT Filed: Aug. 30, 2022
(86) PCT No.: PCT/US2022/075670

§ 371 Date: Feb. 27, 2024

Related U.S. Application Data
(60) Provisional application No. 63/239,298, filed on Aug. 31, 2021.
(60) Provisional application No. 63/356,386, filed on Jun. 28, 2022.

(51) Int. Cl. C12N 15/86; A61K 9/00; A61K 39/00; A61P 31/18; C07K 14/005

(52) U.S. Cl. CPC C12N 15/86; A61K 9/0019; A61P 31/18; C07K 14/005; A61K 39/00; A61K 2039/545; C12N 2710/16143; C12N 2740/16022; C12N 2740/16034; C12N 2740/16222; C12N 2740/16234

(57) ABSTRACT

The disclosure relates to human cytomegalovirus (HCMV) vectors for delivering heterologous antigens and immunogenic compositions comprising the same.

### STATEMENT REGARDING SEQUENCE LISTING

The Sequence Listing associated with this application is provided in XML format in lieu of a paper copy, and is hereby incorporated by reference into the specification. The name of the XML file containing the Sequence Listing is 930485_438WO_SequenceListing.xml. The XML file is 1,189,916 bytes, was created on Aug. 25, 2022, and is being submitted electronically via EFS-Web.

### BACKGROUND

Cytomegalovirus (CMV)-based vaccine vectors have been found to result in strong immune responses to delivered antigens, even for pathogens that have traditionally been able to evade natural immunity and cause repeated or chronic infection. For example, the 68-1 strain of rhesus cytomegalovirus (RhCMV) modified to encode simian immunodeficiency virus (SIV) antigens has been associated with long-lasting protection against SIV challenge (Hansen, S G et al., Immune clearance of highly pathogenic SIV infection. Nature 502, 100-104 (2013); Hansen, S G et al., Profound early control of highly pathogenic SIV by an effector memory T-cell vaccine. Nature 473, 523-527 (2011); Hansen, S G et al., Effector memory T cell responses are associated with protection of rhesus monkeys from mucosal simian immunodeficiency virus challenge. Nat Med. 15, 293-299 (2009)). Subsequent research with CMV vectors revealed that different immune responses could be elicited depending on the specific genetic components of the CMV backbone (Früh, K et al., CD8+ T cell programming by cytomegalovirus vectors: applications in prophylactic and therapeutic vaccination. Curr Opin Immunol. 47, 52-56 (2017); Hansen, S G et al. Cytomegalovirus vectors violate CD8+ T cell epitope recognition paradigms. Science 340, 1237874 (2013)).

The 68-1 of Rhesus cytomegalovirus (RhCMV) has been shown to elicit CD8+ T cells that recognize peptides presented by MHC-II and MHC-E instead of conventional MHC-I. This effect has also been observed in cynomolgus monkey CMV (CyCMV), demonstrating that deletion of the RhCMV and CyCMV homologs of HCMV UL128, UL130, UL146, and UL147 enables the induction of MHC-E-restricted CD8+ T cells (International Application Publication Nos. WO2016/130693A1, WO2018/075591A1). In addition, these vectors elicit MHC-II restricted CD8+ T cells. The induction of MHC-II restricted CD8+ T cells can be eliminated by the insertion of a targeting site for the endothelial cell specific micro RNA (miR) 126 into essential viral genes of these vectors, resulting in “MHC-E only” vectors that exclusively elicit MHC-E restricted CD8+ T cells (International Application Publication No. WO2018/075591A1). In contrast, insertion of the myeloid cell specific miR 142-3p into 68-1 RhCMV has been shown to prevent the induction of MHC-E restricted CD8+ T cells, resulting in vectors that elicit CD8+ T cells exclusively restricted by MHC-II (International Application Publication No. WO2017/087921A1). Deletion of the UL40 homolog Rh67 has also been shown to prevent the induction of MHC-E restricted CD8+ T cells, resulting in “MHC-II-only vectors” (International Application Publication No. WO2016/130693A1). Accordingly, by designing CMV vectors to have particular gene deletions, CMV can be used to deliver antigens and “program” immune responses to those antigens.

According to the World Health Organization, it is estimated that 38 million people worldwide are living with human immunodeficiency virus (HIV) as of 2019, and that approximately 690,000 people will die as a result of HIV/acquired immune deficiency syndrome (AIDS). There is currently no vaccine available for preventing or treating HIV. Additionally, while significant progress has been made in the treatment of HIV/AIDS, people living with HIV still require life-long therapy because existing treatments do not clear latent viral reservoirs (see Erikkson, S et al. Comparative Analysis of Measures of Viral Reservoirs in HIV-1 Eradication Studies. PLOS Pathog 9, e1003174 (2013)). Accordingly, there remains a need for effective preventative or therapeutic vaccines for HIV.

### BRIEF SUMMARY

In some embodiments, the present disclosure provides a recombinant HCMV vector comprising a TR3 backbone and a nucleic acid sequence encoding a heterologous antigen, wherein:
(a) (i) the vector does not express UL18, UL78, UL128, UL130, UL146, or UL147, or orthologs thereof;
   (ii) the vector comprises a nucleic acid sequence encoding UL82, or an ortholog thereof; and
   (iii) the heterologous antigen replaces all or part of UL78 and is operably linked to the UL78 promoter;
(b) (i) the vector does not express UL82, UL128, UL130, UL146, or UL147, or orthologs thereof;
   (ii) the vector comprises a nucleic acid sequence encoding UL18, or an ortholog thereof, and a nucleic acid sequence encoding UL78, or an ortholog thereof; and
   (iii) the heterologous antigen replaces all or part of UL82 and is operably linked to the UL82 promoter; or
(c) (i) the vector does not express UL18, UL82, UL128, UL130, UL146, or UL147, or orthologs thereof;
   (ii) the vector comprises a nucleic acid sequence encoding UL78, or orthologs thereof; and
   (iii) the heterologous antigen replaces all or part of UL82 and is operably linked to the UL82 promoter.

In some embodiments, the heterologous antigen comprises a HIV antigen, e.g., a fusion protein comprising a HIV Gag, a HIV Nef, and a HIV Pol, or immunogenic fragments thereof, or combinations thereof. In some embodiments, the heterologous antigen is or comprises the amino acid sequence according to SEQ ID NO:3 or SEQ ID NO: 4.

In some further embodiments, the present disclosure provides a recombinant HCMV vector comprising a nucleic acid sequence having at least 90%, 91%, 92%, 93%, 94%, 95%, 96%, 97%, 98%, 99%, or 100% identity to the nucleic acid sequence according to SEQ ID NO:7. In some embodiments, the recombinant HCMV vector comprises, consists, or consists essentially of the nucleic acid sequence according to SEQ ID NO:7.

In some further embodiments, the present disclosure provides a recombinant HCMV vector comprising a nucleic acid sequence having at least 90%, 91%, 92%, 93%, 94%, 95%, 96%, 97%, 98%, 99%, or 100% identity to the nucleic acid sequence according to SEQ ID NO:9. In some embodiments, the recombinant HCMV vector comprises, consists, or consists essentially of the nucleic acid sequence according to SEQ ID NO:9.

The present disclosure also provides in some embodiments a recombinant HCMV vector comprising a nucleic acid sequence having at least 90%, at least 91%, at least 92%, at least 93%, at least 94%, at least 95%, at least 96%, at least 97%, at least 98%, at least 99%, or at least 100% identity to the nucleic acid sequence according to SEQ ID NO:5. In some embodiments, the recombinant HCMV vector comprises, consists, or consists essentially of the nucleic acid sequence according to SEQ ID NO:5.

In some embodiments, the present disclosure provides a recombinant HCMV vector comprising a nucleic acid sequence having at least 90%, at least 91%, at least 92%, at least 93%, at least 94%, at least 95%, at least 96%, at least 97%, at least 98%, at least 99%, or at least 100% identity to the nucleic acid sequence according to SEQ ID NO: 6. In some embodiments, the recombinant HCMV vector comprises, consists, or consists essentially of the nucleic acid sequence according to SEQ ID NO:6.

In some embodiments, the present disclosure provides a recombinant HCMV vector comprising a nucleic acid sequence having at least 90%, at least 91%, at least 92%, at least 93%, at least 94%, at least 95%, at least 96%, at least 97%, at least 98%, at least 99%, or at least 100% identity to the nucleic acid sequence according to SEQ ID NO: 8. In some embodiments, the recombinant HCMV vector comprises, consists, or consists essentially of the nucleic acid sequence according to SEQ ID NO:8.

### BRIEF DESCRIPTION OF THE DRAWINGS

FIG. 1 shows cohort dose escalation in the clinical evaluation of an HCMV-based HIV vaccine. Cohort 1 will consist of 6 subjects randomized 4:2 to vaccine or placebo. Cohort 2 will consist of 8 subjects randomized 6:2 to vaccine or placebo. Cohort 3 will consist of 12 subjects randomized 10:2 to vaccine or placebo. The initial starting dose will be 1×10³ focus forming units (ffu). The dose in subsequent cohorts will be increased stepwise in approximately 30-fold increments up to 1×10⁶ ffu based on safety data through 8 weeks. Subjects will receive a second subcutaneous dose on Day 57. The second dose will be the same product dosage level received during the first dose.

FIGS. 2A-2F show the Schedule of Assessments (SoA) used in the clinical evaluation of an HCMV-based HIV vaccine.

FIG. 3 shows the list of laboratory assessments used in clinical evaluation of an HCMV-based HIV vaccine.

FIG. 4 shows grading of adverse event (AE) severity in the clinical evaluation of an HCMV-based HIV vaccine.

FIG. 5 shows the dose schedule for CMV seropositive (“CMV (+)”) and CMV seronegative (“CMV (−)”) subjects receiving either Vector 2 or Vector 3. CMV seronegative subjects will receive escalating doses of Vector 2 or Vector 3 starting at a 5×10⁴ ffu dose and stepwise progression to a higher dose level (5×10⁵ ffu or 5×10⁶ ffu) will be initiated based on safety data through 8 weeks. CMV seropositive subjects will receive either Vector 2 or Vector 3 at a dose of 5×10⁴ ffu, 5×10⁵ ffu or 5×10⁶ ffu, wherein all three cohorts will be dosed concurrently.

FIGS. 6A-6E show the development of vector construction for the CMV vector backbone. FIG. 6A shows the US (unique short) region of the HCMV TR where the BAC cassette was inserted, in addition to the mutated UL97 gene conferring resistance to ganciclovir. FIG. 6B shows insertion of the BAC cassette necessary for propagation in E. coli between US1 and US7, thereby deleting US2-US6. FIG. 6C shows insertion of US2-US7 from HCMV strain AD169, GFP, and LoxP sites and the consequential deletion of US7 from TR. FIG. 6D shows replacement of TR UL97 by AD169 UL97 to restore ganciclovir sensitivity, removal of the GFP gene, and addition of Cre recombinase to the BAC cassette under the control of a SV40 early promoter. FIG. 6E shows excision of the BAC cassette upon reconstitution of the virus, leaving a single 34-bp LoxP site located between US7 and US8 as the only remaining non-viral sequence in the virus genome.

FIG. 7 shows the manufacturing process for generating the master virus seed and clinical trial material for Vector 2 and Vector 3.

FIG. 8 shows a comparison of hold time in two bioprocessing bag types, CX5-14 Labtainer™ PE (polyethylene) and Flexboy® EVA (ethylene vinyl acetate) over 72 hours at 2-8° C.

FIG. 9 shows titers following a cumulative hold time study. Representative intermediate bulk formulated in histidine trehalose (HT) buffer was held in a Flexboy® EVA bag, filled to 30% of capacity, overnight (“O/N”) at 2-8° C. for 16 hours (labeled “#2”). Following the overnight hold, intermediate bulk was held out to 72 hours at room temperature (RT) (labeled “#3” to “#5”), after which it was filled at 0.7 mL in vials and held for an additional 48 hours at RT (labeled “#6” and “#7”) to mimic a worst-case scenario for RT holds.

### DETAILED DESCRIPTION

### Glossary

The following sections provide a detailed description of CMV vectors, and related pharmaceutical compositions and methods of inducing an immune response, such as an anti-HIV immune response, and methods of treating or preventing disease (e.g., HIV). Prior to setting forth this disclosure in more detail, it may be helpful to an understanding thereof to provide definitions of certain terms to be used herein. Additional definitions are set forth throughout this disclosure.

Unless the context requires otherwise, throughout the present specification and claims, the word “comprise” and variations thereof, such as “comprises” and “comprising,” are to be construed in an open, inclusive sense, that is, as “including, but not limited to”. “Consisting of” shall mean excluding more than trace elements of other ingredients and substantial method steps disclosed herein, and in the case of an amino acid or nucleic acid sequence, excluding additional amino acids or nucleotides, respectively. The term “consisting essentially of” limits the scope of a claim to the specified materials or steps, or to those that do not materially affect the basic characteristics of a claimed invention. For example, a composition consisting essentially of the elements as defined herein would not exclude trace contaminants from the isolation and purification method and pharmaceutically acceptable carriers, such as phosphate buffered saline, preservatives, and the like. Similarly, a protein consists essentially of a particular amino acid sequence when the protein includes additional amino acids that contribute to at most 20% of the length of the protein and do not substantially affect the activity of the protein (e.g., alters the activity of the protein by no more than 50%). Embodiments defined by each of the transitional terms are within the scope of this invention.

In the present description, the term “about” means ±20% of the indicated range, value, or structure, unless otherwise indicated.

It should be understood that the terms “a” and “an” as used herein include “one or more” of the enumerated components unless stated otherwise. The use of the alternative (e.g., “or”) should be understood to mean either one, both, or any combination thereof of the alternatives, and may be used synonymously with “and/or”. As used herein, the terms “include” and “have” are used synonymously, which terms and variants thereof are intended to be construed as non-limiting.

The word “substantially” does not exclude “completely”; e.g., a composition which is “substantially free” from Y may be completely free from Y. Where necessary, the word “substantially” may be omitted from definitions provided herein.

As used herein, the terms “peptide”, “polypeptide”, and “protein” and variations of these terms refer to a molecule, in particular a peptide, oligopeptide, polypeptide, or protein including fusion protein, respectively, comprising at least two amino acids joined to each other by a normal peptide bond, or by a modified peptide bond, such as for example in the cases of isosteric peptides. For example, a peptide, polypeptide, or protein may be composed of amino acids selected from the 20 amino acids defined by the genetic code, linked to each other by a normal peptide bond (“classical” polypeptide). A peptide, polypeptide, or protein can be composed of L-amino acids and/or D-amino acids. In particular, the terms “peptide”, “polypeptide”, and “protein” also include “peptidomimetics,” which are defined as peptide analogs containing non-peptidic structural elements, which peptides are capable of mimicking or antagonizing the biological action(s) of a natural parent peptide. A peptidomimetic lacks classical peptide characteristics such as enzymatically scissile peptide bonds. In particular, a peptide, polypeptide, or protein may comprise amino acids other than the 20 amino acids defined by the genetic code in addition to these amino acids, or it can be composed of amino acids other than the 20 amino acids defined by the genetic code. In particular, a peptide, polypeptide, or protein in the context of the present disclosure can equally be composed of amino acids modified by natural processes, such as post-translational maturation processes or by chemical processes, which are well known to a person skilled in the art. Such modifications are fully detailed in the literature. These modifications can appear anywhere in the polypeptide: in the peptide skeleton, in the amino acid chain, or even at the carboxy- or amino-terminal ends. In particular, a peptide or polypeptide can be branched following an ubiquitination or be cyclic with or without branching. This type of modification can be the result of natural or synthetic post-translational processes that are well known to a person skilled in the art. The terms “peptide”, “polypeptide”, or “protein” in the context of the present disclosure in particular also include modified peptides, polypeptides, and proteins. For example, peptide, polypeptide, or protein modifications can include acetylation, acylation, ADP-ribosylation, amidation, covalent fixation of a nucleotide or of a nucleotide derivative, covalent fixation of a lipid or of a lipidic derivative, the covalent fixation of a phosphatidylinositol, covalent or non-covalent cross-linking, cyclization, disulfide bond formation, demethylation, glycosylation including pegylation, hydroxylation, iodization, methylation, myristoylation, oxidation, proteolytic processes, phosphorylation, prenylation, racemization, seneloylation, sulfatation, amino acid addition such as arginylation, or ubiquitination. Such modifications are fully detailed in the literature (Proteins Structure and Molecular Properties, 2nd Ed., T. E. Creighton, New York (1993); Post-translational Covalent Modifications of Proteins, B. C. Johnson, Ed., Academic Press, New York (1983); Seifter, et al., Analysis for protein modifications and nonprotein cofactors, Meth. Enzymol. 182:626-46 (1990); and Rattan, et al., Protein Synthesis: Post-translational Modifications and Aging, Ann NY Acad Sci 663:48-62 (1992)). Accordingly, the terms “peptide”, “polypeptide”, and “protein” include for example lipopeptides, lipoproteins, glycopeptides, glycoproteins, and the like.

“Orthologs” of proteins are typically characterized by possession of greater than 75% sequence identity counted over the full-length alignment with the amino acid sequence of specific protein using an alignment algorithm, for example, the ALIGN program (version 2.0) set to default parameters. Proteins with even greater similarity to a reference sequence will show increasing percentage identities when assessed by this method, such as at least 80%, at least 85%, at least 90%, at least 92%, at least 95%, or at least 98% sequence identity. In addition, sequence identity can be compared over the full length of particular domains of the disclosed peptides.

The term “homologous” or “homolog” refers to a molecule or activity found in or derived from a host cell, species, or strain. For example, a heterologous or exogenous molecule or gene encoding the molecule may be homologous to a native host or host cell molecule or gene that encodes the molecule, respectively, but may have an altered structure, sequence, expression level or combinations thereof.

As used herein a “(poly) peptide” comprises a single chain of amino acid monomers linked by peptide bonds as explained above. A “protein”, as used herein, comprises one or more, e.g., 1, 2, 3, 4, 5, 6, 7, 8, 9 or 10 (poly) peptides, i.e., one or more chains of amino acid monomers linked by peptide bonds as explained above. In particular embodiments, a protein according to the present disclosure comprises 1, 2, 3, or 4 polypeptides.

As used herein, the terms “nucleic acid”, “nucleic acid molecule,” “nucleic acid sequence,” and “polynucleotide” are used interchangeably and are intended to include DNA molecules and RNA molecules, including, without limitation, messenger RNA (mRNA), DNA/RNA hybrids, or synthetic nucleic acids. The nucleic acid may be single-stranded, or partially or completely double stranded (duplex). Duplex nucleic acids may be homoduplex or heteroduplex. A nucleic acid molecule may be single-stranded or double-stranded.

As used herein, the term “coding sequence” is intended to refer to a polynucleotide molecule, which encodes the amino acid sequence of a protein product. The boundaries of the coding sequence are generally determined by an open reading frame, which usually begins with an ATG start codon.

The term “expression” as used herein refers to any step involved in the production of the polypeptide, including transcription, post-transcriptional modification, translation, post-translational modification, secretion, or the like.

As used herein, the term “sequence variant” refers to any sequence having one or more alterations in comparison to a reference sequence, whereby a reference sequence is any of the sequences listed in the sequence listing, i.e., SEQ ID NO: 1 to SEQ ID NO:9. Thus, the term “sequence variant” includes nucleotide sequence variants and amino acid sequence variants. For a sequence variant in the context of a nucleotide sequence, the reference sequence is also a nucleotide sequence, whereas for a sequence variant in the context of an amino acid sequence, the reference sequence is also an amino acid sequence. A “sequence variant” as used herein is at least 80%, at least 85%, at least 90%, at least 95%, at least 98%, or at least 99% identical to the reference sequence. Sequence identity is usually calculated with regard to the full length of the reference sequence (i.e., the sequence recited in the application), unless otherwise specified. Percentage identity, as referred to herein, can be determined, for example, using various methods of alignment known in the art, such as BLAST using the default parameters specified by the NCBI (the National Center for Biotechnology Information; http://www.ncbi.nlm.nih.gov/) [Blosum 62 matrix; gap open penalty=1 1 and gap extension penalty=1]. A “sequence variant” in the context of a nucleic acid (nucleotide) sequence has an altered sequence in which one or more of the nucleotides in the reference sequence is deleted, or substituted, or one or more nucleotides are inserted into the sequence of the reference nucleotide sequence. Nucleotides are referred to herein by the standard one-letter designation (A, C, G, or T). Due to the degeneracy of the genetic code, a “sequence variant” of a nucleotide sequence can either result in a change in the respective reference amino acid sequence, i.e., in an amino acid “sequence variant” or not. In certain embodiments, the nucleotide sequence variants are variants that do not result in amino acid sequence variants (i.e., silent mutations). However, nucleotide sequence variants leading to “non-silent” mutations are also within the scope, in particular such nucleotide sequence variants, which result in an amino acid sequence, which is at least 80%, at least 85%, at least 90%, at least 95%, at least 98%, or at least 99% identical to the reference amino acid sequence. A “sequence variant” in the context of an amino acid sequence has an altered sequence in which one or more of the amino acids is deleted, substituted or inserted in comparison to the reference amino acid sequence. As a result of the alterations, such a sequence variant has an amino acid sequence which is at least 80%, at least 85%, at least 90%, at least 95%, at least 98%, or at least 99% identical to the reference amino acid sequence. For example, per 100 amino acids of the reference sequence a variant sequence having no more than 10 alterations, i.e., any combination of deletions, insertions, or substitutions, is “at least 90% identical” to the reference sequence.

While it is possible to have non-conservative amino acid substitutions, in certain embodiments, the substitutions are conservative amino acid substitutions, in which the substituted amino acid has similar structural or chemical properties with the corresponding amino acid in the reference sequence. By way of example, conservative amino acid substitutions involve substitution of one aliphatic or hydrophobic amino acids, e.g., alanine, valine, leucine, and isoleucine, with another; substitution of one hydroxyl-containing amino acid, e.g., serine and threonine, with another; substitution of one acidic residue, e.g., glutamic acid or aspartic acid, with another; replacement of one amide-containing residue, e.g., asparagine and glutamine, with another; replacement of one aromatic residue, e.g., phenylalanine and tyrosine, with another; replacement of one basic residue, e.g., lysine, arginine, and histidine, with another; and replacement of one small amino acid, e.g., alanine, serine, threonine, methionine, and glycine, with another.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include the fusion to the N- or C-terminus of an amino acid sequence to a reporter molecule or an enzyme.

Unless otherwise stated, alterations in the sequence variants do not abolish the functionality of the respective reference sequence, for example, in the present case, the functionality of an antigen or vector disclosed herein. Guidance in determining which nucleotides and amino acid residues, respectively, may be substituted, inserted, or deleted without abolishing such functionality can be found by using computer programs well known in the art.

The nucleotide sequences of the present disclosure may be codon optimized, for example the codons may be optimized for use in human cells. For example, any viral or bacterial sequence may be so altered. Many viruses, including HIV and other lentiviruses, use a large number of rare codons and, by altering these codons to correspond to codons commonly used in the desired subject, enhanced expression of antigens may be achieved as described in André, S et al. (Increased Immune Response Elicited by DNA Vaccination with a Synthetic gp120 Sequence with Optimized Codon Usage. J Virol. 72, 1497-1503 (1998)).

As used herein, a nucleic acid sequence or an amino acid sequence “derived from” a designated nucleic acid, peptide, polypeptide, or protein refers to the origin of the nucleic acid, peptide, polypeptide, or protein. In some embodiments, the nucleic acid sequence or amino acid sequence which is derived from a particular sequence has an amino acid sequence that is essentially identical to that sequence or a portion thereof, from which it is derived, whereby “essentially identical” includes sequence variants as defined above. In certain embodiments, the nucleic acid sequence or amino acid sequence which is derived from a particular peptide or protein is derived from the corresponding domain in the particular peptide or protein. Thereby, “corresponding” refers in particular to the same functionality. For example, an “extracellular domain” corresponds to another “extracellular domain” (of another protein), or a “transmembrane domain” corresponds to another “transmembrane domain” (of another protein). “Corresponding” parts of peptides, proteins, and nucleic acids are thus identifiable to one of ordinary skill in the art. Likewise, sequences “derived from” other sequences are usually identifiable to one of ordinary skill in the art as having its origin in the sequence.

In some embodiments, a nucleic acid sequence or an amino acid sequence derived from another nucleic acid, peptide, polypeptide, or protein may be identical to the starting nucleic acid, peptide, polypeptide, or protein (from which it is derived). However, a nucleic acid sequence or an amino acid sequence derived from another nucleic acid, peptide, polypeptide, or protein may also have one or more mutations relative to the starting nucleic acid, peptide, polypeptide, or protein (from which it is derived), in particular a nucleic acid sequence or an amino acid sequence derived from another nucleic acid, peptide, polypeptide, or protein may be a functional sequence variant as described above of the starting nucleic acid, peptide, polypeptide, or protein (from which it is derived). For example, in a peptide/protein one or more amino acid residues may be substituted with other amino acid residues or one or more amino acid residue insertions or deletions may occur.

As used herein, the term “mutation” relates to a change in the nucleic acid sequence and/or in the amino acid sequence in comparison to a reference sequence, e.g., a corresponding genomic sequence. A mutation, e.g., in comparison to a genomic sequence, may be, for example, a (naturally occurring) somatic mutation, a spontaneous mutation, an induced mutation, e.g., induced by enzymes, chemicals, or radiation, or a mutation obtained by site-directed mutagenesis (molecular biology methods for making specific and intentional changes in the nucleic acid sequence and/or in the amino acid sequence). Thus, the terms “mutation” or “mutating” shall be understood to also include physically making a mutation, e.g., in a nucleic acid sequence or in an amino acid sequence. A mutation includes substitution, deletion, and insertion of one or more nucleotides or amino acids as well as inversion of several successive nucleotides or amino acids. Some types of coding sequence mutations include point mutations (differences in individual nucleotides or amino acids); silent mutations (differences in nucleotides that do not result in an amino acid changes); deletions (differences in which one or more nucleotides or amino acids are missing, up to and including a deletion of the entire coding sequence of a gene); frameshift mutations (differences in which deletion of a number of nucleotides indivisible by 3 results in an alteration of the amino acid sequence). A mutation that results in a difference in an amino acid may also be called an amino acid substitution mutation. Amino acid substitution mutations may be described by the amino acid change relative to wild type at a particular position in the amino acid sequence. To achieve a mutation in an amino acid sequence, a mutation may be introduced into the nucleotide sequence encoding said amino acid sequence in order to express a (recombinant) mutated polypeptide. A mutation may be achieved, e.g., by altering, e.g., by site-directed mutagenesis, a codon of a nucleic acid molecule encoding one amino acid to result in a codon encoding a different amino acid, or by synthesizing a sequence variant, e.g., by knowing the nucleotide sequence of a nucleic acid molecule encoding a polypeptide and by designing the synthesis of a nucleic acid molecule comprising a nucleotide sequence encoding a variant of the polypeptide without the need for mutating one or more nucleotides of a nucleic acid molecule.

The term “recombinant”, as used herein (e.g., a recombinant protein, a recombinant nucleic acid, a recombinant antibody, etc.), refers to any molecule (protein, nucleic acid, antibody, etc.) that is prepared, expressed, created, or isolated by recombinant means, and which is not naturally occurring. With reference to a nucleic acid or polypeptide, “recombinant” refers to one that has a sequence that is not naturally occurring or has a sequence that is made by an artificial combination of two or more otherwise separated segments of sequence, for example a CMV vector comprising a heterologous antigen. This artificial combination is often accomplished by chemical synthesis or, more commonly, by the artificial manipulation of isolated segments of nucleic acids, e.g., by genetic engineering techniques. A recombinant polypeptide may also refer to a polypeptide that has been made using recombinant nucleic acids, including recombinant nucleic acids transferred to a host organism that is not the natural source of the polypeptide (for example, nucleic acids encoding polypeptides that form a CMV vector comprising a heterologous antigen).

As used herein, the term “vector” refers to a carrier by which into which nucleic acid molecules of particular sequence can be incorporated and then introduced into a host cell, thereby producing a transformed host cell. A vector may include nucleic acid sequences that permit it to replicate in a host cell, such as an origin of replication. A vector may also include one or more selectable marker genes and other genetic elements known in the art, including promoter elements that direct nucleic acid expression. Vectors can be viral vectors, such as CMV vectors. Viral vectors may be constructed from wild type or attenuated virus, including replication deficient virus.

As the term “operably linked” is used herein, a first nucleic acid sequence is operably linked with a second nucleic acid sequence when the first nucleic acid sequence is placed in such a way that it has an effect upon the second nucleic acid sequence. Operably linked DNA sequences may be contiguous, or they may operate at a distance.

As used herein, the term “promoter” may refer to any of a number of nucleic acid control sequences that directs transcription of a nucleic acid. Typically, a eukaryotic promoter includes necessary nucleic acid sequences near the start site of transcription, such as, in the case of a polymerase II type promoter, a TATA element or any other specific DNA sequence that is recognized by one or more transcription factors. Expression by a promoter may be further modulated by enhancer or repressor elements. Numerous examples of promoters are available and well known to those of ordinary skill in the art. A nucleic acid comprising a promoter operably linked to a nucleic acid sequence that codes for a particular polypeptide may be termed an expression vector.

As used herein, the terms “cell,” “cell line,” and “cell culture” are used interchangeably and all such designations include progeny. Thus, the words “transformants” and “transformed cells” include the primary subject cell and cultures derived therefrom without regard for the number of transfers. It is also understood that all progeny may not be precisely identical in DNA content, due to deliberate or inadvertent mutations. Variant progeny that have the same function or biological activity as screened for in the originally transformed cell are included. Where distinct designations are intended, it will be clear from the context.

As used herein, the term “microRNA” refers to a major class of biomolecules involved in control of gene expression. For example, in human heart, liver, or brain, miRNAs play a role in tissue specification or cell lineage decisions. In addition, miRNAs influence a variety of processes, including early development, cell proliferation and cell death, and apoptosis and fat metabolism. The large number of miRNA genes, the diverse expression patterns, and the abundance of potential miRNA targets suggest that miRNAs may be a significant source of genetic diversity. A mature miRNA is typically an 8-25 nucleotide non-coding RNA that regulates expression of an mRNA including sequences complementary to the miRNA. These small RNA molecules are known to control gene expression by regulating the stability and/or translation of mRNAs. For example, miRNAs bind to the 3′ UTR of target mRNAs and suppress translation. MiRNAs may also bind to target mRNAs and mediate gene silencing through the RNAi pathway. MiRNAs may also regulate gene expression by causing chromatin condensation.

A miRNA silences translation of one or more specific mRNA molecules by binding to a miRNA recognition element (MRE) which is defined as any sequence that directly base pairs with and interacts with the miRNA somewhere on the mRNA transcript. Often, the MRE is present in the 3′ untranslated region (UTR) of the mRNA, but it may also be present in the coding sequence or in the 5′ UTR. MREs are not necessarily perfect complements to miRNAs, usually having only a few bases of complementarity to the miRNA and often containing one or more mismatches within those bases of complementarity. The MRE may be any sequence capable of being bound by a miRNA sufficiently that the translation of a gene to which the MRE is operably linked (such as a CMV gene that is essential or augmenting for growth in vivo) is repressed by a miRNA silencing mechanism such as the RISC.

The term “vaccine” as used herein is typically understood to be a prophylactic or therapeutic material providing at least one antigen or immunogen. The antigen or immunogen may be derived from any material that is suitable for vaccination. For example, the antigen or immunogen may be derived from a pathogen, such as from bacteria or virus particles, etc., or from a tumor or cancerous tissue. The antigen or immunogen stimulates the body's adaptive immune system to provide an adaptive immune response. In particular, an “antigen” or an “immunogen” refers typically to a substance which may be recognized by the immune system (e.g., the adaptive immune system), and which is capable of triggering an antigen-specific immune response, e.g., by formation of antibodies and/or antigen-specific T cells as part of an adaptive immune response. Typically, an antigen may be or may comprise a peptide or protein which may be presented by the MHC to T-cells. Vaccines can be used prophylactically or therapeutically. Thus, vaccines can be used reduce the likelihood of developing a disease (such as a tumor or pathological infection) or to reduce the severity of symptoms of a disease or condition, limit the progression of the disease or condition (such as a tumor or a pathological infection), or limit the recurrence of a disease or condition (such as a tumor). In particular embodiments, a vaccine comprises a replication-deficient CMV expressing a heterologous antigen, such as an HIV antigen.

As used herein, the terms “antigen” or “immunogen” are used interchangeably to refer to a substance, typically a protein, which is capable of inducing an immune response in a subject. The term also refers to proteins that are immunologically active in the sense that once administered to a subject (either directly or by administering to the subject a nucleotide sequence or vector that encodes the protein) the protein is able to evoke an immune response of the humoral and/or cellular type directed against that protein.

As used herein, the term “heterologous antigen” refers to any protein or fragment thereof that is not derived from CMV. Heterologous antigens may be pathogen-specific antigens, tumor virus antigens, tumor antigens, host self-antigens, or any other antigen.

As used herein, “antigen-specific T cell” refers to a CD8+ or CD4+ lymphocyte that recognizes a particular antigen. Generally, antigen-specific T cells specifically bind to a particular antigen presented by MHC molecules, but not other antigens presented by the same MHC.

As used herein, “immunogenic peptide” refers to peptide that comprises an allele-specific motif or other sequence, such as an N-terminal repeat, such that the peptide will bind an MHC molecule and induce a cytotoxic T lymphocyte (“CTL”) response, or a B cell response (for example, antibody production) against the antigen from which the immunogenic peptide is derived. In some embodiments, immunogenic peptides are identified using sequence motifs or other methods, such as neural net or polynomial determinations known in the art. Typically, algorithms are used to determine the “binding threshold” of peptides to select those with scores that give them a high probability of binding at a certain affinity and will be immunogenic. The algorithms are based either on the effects on MHC binding of a particular amino acid at a particular position, the effects on antibody binding of a particular amino acid at a particular position, or the effects on binding of a particular substitution in a motif-containing peptide. Within the context of an immunogenic peptide, a “conserved residue” is one which appears in a significantly higher frequency than would be expected by random distribution at a particular position in a peptide. In some embodiments, a conserved residue is one where the MHC structure may provide a contact point with the immunogenic peptide.

As used herein, the term “administration” means to provide or give a subject an agent, such as a composition comprising an effective amount of a CMV vector comprising an exogenous antigen by any effective route. Exemplary routes of administration include, but are not limited to, injection (such as subcutaneous, intramuscular, intradermal, intraperitoneal, and intravenous), oral, sublingual, rectal, transdermal, intranasal, vaginal and inhalation routes.

As used herein, a “pharmaceutically acceptable carrier” of use is conventional. Remington's Pharmaceutical Sciences, by E. W. Martin, Mack Publishing Co., Easton, PA, 19th Edition, 1995, describes compositions and formulations suitable for pharmaceutical delivery of the compositions disclosed herein. In general, the nature of the carrier will depend on the particular mode of administration being employed. For instance, parenteral formulations usually comprise injectable fluids that include pharmaceutically and physiologically acceptable fluids such as water, physiological saline, balanced salt solutions, buffers, aqueous dextrose, glycerol, or the like as a vehicle. For solid compositions (such as powder, pill, tablet, or capsule forms), conventional non-toxic solid carriers may include, for example, pharmaceutical grades of mannitol, lactose, starch, or magnesium stearate. In addition to biologically neutral carriers, pharmaceutical compositions to be administered may contain minor amounts of non-toxic auxiliary substances, such as wetting or emulsifying agents, preservatives, and pH buffering agents and the like, for example sodium acetate or sorbitan monolaurate.

Doses are often expressed in relation to bodyweight. Thus, a dose which is expressed as [g, mg, or other unit]/kg (or g, mg, etc.) usually refers to [g, mg, or other unit] “per kg (or g, mg, etc.) bodyweight”, even if the term “bodyweight” is not explicitly mentioned.

Doses may be expressed as focus-forming units (ffu) per ml as determined by a focus-forming assay in which areas (foci) of cytopathic effect that indicate replication of the virus on a lawn of cells are counted.

The term “disease” as used herein is intended to be generally synonymous, and is used interchangeably with, the terms “disorder” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

### Antigens

### HIV Fusion Antigens

Disclosed herein are fusion proteins comprising HIV antigens and nucleic acids encoding the same.

In some embodiments, the present disclosure provides a fusion protein comprising one or more of HIV Gag, HIV Nef, and HIV Pol, or portions thereof. In some embodiments, the fusion antigen comprises an amino acid sequence having at least 90%, at least 91%, at least 92%, at least 93%, at least 94%, at least 95%, at least 96%, at least 97%, at least 98%, at least 99%, or 100% identity to the amino acid sequence according to SEQ ID NO:3. In some embodiments, the fusion antigen comprises an amino acid sequence having at least 90%, at least 91%, at least 92%, at least 93%, at least 94%, at least 95%, at least 96%, at least 97%, at least 98%, at least 99%, or 100% identity to the amino acid sequence according to SEQ ID NO:4. In some embodiments, the fusion antigen comprises the amino acid sequence according to SEQ ID NO: 3. In some embodiments, the fusion antigen comprises the amino acid sequence according to SEQ ID NO:4. In some embodiments, the fusion antigen consists of the amino acid sequence according to SEQ ID NO:3. In some embodiments, the fusion antigen consists of the amino acid sequence according to SEQ ID NO:4. In some embodiments, the fusion antigen comprises amino acids 2-912 of the amino acid sequence according to SEQ ID NO:3. In some embodiments, the fusion antigen comprises amino acids 2-911 of the amino acid sequence according to SEQ ID NO:4. In some embodiments, the fusion antigen consists of amino acids 2-912 of the amino acid sequence according to SEQ ID NO:3. In some embodiments, the fusion antigen consists of amino acids 2-911 of the amino acid sequence according to SEQ ID NO:4.

In some embodiments, the present disclosure provides nucleic acid molecules encoding the fusion proteins described above, for example, a nucleic acid molecule according to SEQ ID NO: 1 or SEQ ID NO:2. In some embodiments, the nucleic acid molecule encoding the fusion protein comprises a nucleic acid sequence having at least 90%, at least 91%, at least 92%, at least 93%, at least 94%, at least 95%, at least 96%, at least 97%, at least 98%, at least 99%, or 100% identity to the nucleic acid sequence according to SEQ ID NO:1. In some embodiments, the nucleic acid molecule encoding the fusion protein comprises a nucleic acid sequence having at least 90%, at least 91%, at least 92%, at least 93%, at least 94%, at least 95%, at least 96%, at least 97%, at least 98%, at least 99%, or 100% identity to the nucleic acid sequence according to SEQ ID NO: 2. In some embodiments, the present disclosure provides a nucleic acid molecule comprising the sequence according to SEQ ID NO:1. In some embodiments, the present disclosure provides a nucleic acid molecule comprising the sequence according to SEQ ID NO: 2. In some embodiments, the present disclosure provides a nucleic acid molecule consisting of the sequence according to SEQ ID NO:1. In some embodiments, the present disclosure provides a nucleic acid molecule consisting of sequence according to SEQ ID NO:2.

In some embodiments, the present disclosure provides vectors encoding a fusion protein as described above. For example, in some embodiments, the present disclosure provides a vector comprising a nucleic acid sequence according to SEQ ID NO: 1 or SEQ ID NO:2. In some embodiments, the present disclosure provides a vector encoding a fusion protein, wherein the fusion protein comprises the amino acid sequence according to SEQ ID NO:3. In some embodiments, the present disclosure provides a vector encoding a fusion protein, wherein the fusion protein comprises the amino acid sequence according to SEQ ID NO:4. In some embodiments, the present disclosure provides a vector encoding a fusion protein, wherein the fusion protein consists of the amino acid sequence according to SEQ ID NO:3. In some embodiments, the present disclosure provides a vector encoding a fusion protein, wherein the fusion protein consists of the amino acid sequence according to SEQ ID NO:4.

The vector may be any expression vector known in the art. For the antigens to be expressed, the protein coding sequence of the fusion protein should be “operably linked” to regulatory or nucleic acid control sequences that direct transcription and translation of the protein. A coding sequence and a nucleic acid control sequence or promoter are said to be “operably linked” when they are covalently linked in such a way as to place the expression or transcription and/or translation of the coding sequence under the influence or control of the nucleic acid control sequence. The “nucleic acid control sequence” may be any nucleic acid element, such as, but not limited to promoters, enhancers, IRES, introns, and other elements described herein that direct the expression of a nucleic acid sequence or coding sequence that is operably linked thereto. “Promoter” refers to a group of transcriptional control modules that are clustered around the initiation site for RNA polymerase II and that when operationally linked to the protein coding sequences of the disclosure lead to the expression of the encoded protein. The expression of heterologous antigens and fusion proteins of the present disclosure may be under the control of a constitutive promoter or of an inducible promoter, which initiates transcription only when exposed to some particular external stimulus, such as, without limitation, antibiotics such as tetracycline, hormones such as ecdysone, or heavy metals. The promoter may also be specific to a particular cell-type, tissue, or organ. Many suitable promoters and enhancers are known in the art, and any such suitable promoter or enhancer may be used for expression of the transgenes of the disclosure. For example, suitable promoters and/or enhancers may be selected from the Eukaryotic Promoter Database (EPDB).

In some embodiments, the vector encoding the fusion protein is a plasmid, bacterial vector, or viral vector. In some embodiments, the vector is a viral vector, such a poxvirus, adenovirus, rubella, sendai virus, rhabdovirus, alphavirus, herpesvirus, or adeno-associated virus. In some embodiments, the vector encoding the fusion protein is a CMV vector, e.g., a RhCMV or HCMV vector. In some embodiments, the vector encoding the fusion protein is a recombinant HCMV vector comprising a TR3 backbone.

In some embodiments, the present disclosure provides methods of generating an immune response to HIV or preventing or treating HIV in a subject comprising administering a vector encoding the fusion protein as described above.

The present disclosure also provides vaccines comprising RNA or proteins based on the fusion protein described above, and their use in methods of generating an immune response to HIV or preventing or treating HIV in a subject.

### Other Antigens

In some embodiments, the heterologous antigen encoded by an HCMV vector disclosed herein is a pathogen-specific antigen, a tumor antigen, a tumor-specific antigen, or a host self-antigen.

The pathogen-specific antigen may be derived from, for example, human immunodeficiency virus (HIV), simian immunodeficiency virus (SIV), herpes simplex virus type 1, herpes simplex virus type 2, hepatitis B virus, hepatitis C virus, papillomavirus, Plasmodium parasites, Clostridium tetani, or Mycobacterium tuberculosis.

In some embodiments, the pathogen-specific antigen comprises a HIV Env, HIV Tat, HIV Rev, HIV Vif, HIV Vpu, HIV Gag, HIV Nef, or HIV Pol. In some embodiments, the pathogen-specific antigen comprises a fusion protein comprising two or more of HIV Env, HIV Tat, HIV Rev, HIV Vif, HIV Vpu, HIV Gag, HIV Nef, and HIV Pol. In some embodiments, the pathogen-specific antigen comprises an HIV Gag, HIV Nef, or HIV Pol antigen. For example, the antigen may be any HIV antigen sequence or fusion thereof described in International Application Publication No. WO2016/054654A1, which is incorporated herein by reference for its teachings related to HIV antigens.

In some embodiments, the pathogen-specific antigen comprises a Mycobacterium tuberculosis antigen. In some embodiments, the pathogen-specific antigen comprises a fusion protein comprising two or more Mycobacterium tuberculosis antigens. For example, the antigen may be any antigen or fusion thereof described in International Application Publication No. WO2017/223146A1, which is incorporated herein by reference for its teachings related to Mycobacterium tuberculosis antigens. In some embodiments, the pathogen-specific antigen is Ag85A-Ag85B-Rv3407, Rv1733-Rv2626c, RpfA-RpfC-RpfD, Ag85B-ESAT6, or Ag85A-ESAT6-Rv3407-Rv2626c-RpfA-RpfD.

Tumor antigens are relatively restricted to tumor cells and can be any protein that induces an immune response. However, many tumor antigens are host (self) proteins and thus are typically not seen as antigenic by the host immune system. Tumor antigens can also be abnormally expressed by cancer cells. Tumor antigens can also be germline/testis antigens expressed in cancer cells, cell lineage differentiation antigens not expressed in adult tissue, or antigens overexpressed in cancer cells. Tumor antigens include, but are not limited to: prostatic acidic phosphatase (PAP); Wilms tumor suppressor protein (WT1); Mesothelin (MSLN); Her-2 (HER2); human papilloma virus antigen E6 of strain HPV16; human papilloma virus antigen E7 of strain HPV16; human papilloma virus antigen E6 of strain HPV18; Human papilloma virus antigen E7 of strain HPV18; a fusion protein of human papilloma virus E6 and E7 from HPV16 and HPV18; mucin 1 (MUC1); LMP2; epidermal growth factor receptor (EGFR); p53; New York esophagus 1 (NY-ESO-1); prostate specific membrane antigen (PSMA); GD2, carcinoembryonic antigen (CEA); melanoma antigen a/melanoma antigen recognized by T cells 1 (MelanA/MART1); Ras; gp100, Proteinase 3 (PR1), Bcr-abl; Survivin; prostate specific antigen (PSA); human telomerase reverse transcriptase (hTERT); EphA2; ML-IAP; alphafetoprotein (AFP); EpCAM; ERG; NA17; PAX3; ALK; Androgen receptor (AR); Cyclin B1; MYCN; RhoC; tyrosine related protein 2 (TRP-2); GD3; Fucosyl GM1; PSCA; sLe (a); CYP1B1; PLCA1; GM3; BORIS; Tn; GloboH; Ets variant gene 6/acute myeloid leukemia 1 gene ETS (ETV6-AML); NY-BR-1; RGS5; squamous antigen rejecting tumor or 3 (SART3); STn; Carbonic anhydrase IX; PAX5; OY-TES1; Sperm protein 17; LCK; HMWMAA; AKAP-4; SSX2; B7H3; Legumain; Tie 2; Page4; VEGFR2; MAD-CT-1; FAP; PDGFR; MAD-CT-2; Fosrelated antigen 1; TAG-72; 9D7; EphA3; Telomerase; SAP-1; BAGE family; CAGE family; GAGE family; MAGE family; SAGE family; XAGE family; preferentially expressed antigen of melanoma (PRAME); melanocortin 1 receptor (MC1R); β-catenin; BRCA1/2; CDK4; chronic myelogenous leukemia 66 (CML66); and TGF-β. In certain embodiments, the host self-antigens include prostatic acidic phosphatase, Wilms tumor suppressor protein, mesothelin, or Her-2.

In some embodiments the tumor antigen is derived from a cancer. The cancer includes, but is not limited to: Acute lymphoblastic leukemia; Acute myeloid leukemia; Adrenocortical carcinoma; AIDS-related cancers; AIDS-related lymphoma; Anal cancer; Appendix cancer; Astrocytoma, childhood cerebellar or cerebral; Basal cell carcinoma; Bile duct cancer, extrahepatic; Bladder cancer; Bone cancer, Osteosarcoma/Malignant fibrous histiocytoma; Brainstem glioma; Brain tumor; Brain tumor, cerebellar astrocytoma; Brain tumor, cerebral astrocytoma/malignant glioma; Brain tumor, ependymoma; Brain tumor, medulloblastoma; Brain tumor, supratentorial primitive neuroectodermal tumors; Brain tumor, visual pathway and hypothalamic glioma; Breast cancer; Bronchial adenomas/carcinoids; Burkitt lymphoma; Carcinoid tumor, childhood; Carcinoid tumor, gastrointestinal; Carcinoma of unknown primary; Central nervous system lymphoma, primary; Cerebellar astrocytoma, childhood; Cerebral astrocytoma/Malignant glioma, childhood; Cervical cancer; Childhood cancers; Chronic lymphocytic leukemia; Chronic myelogenous leukemia; Chronic myeloproliferative disorders; Colon Cancer; Cutaneous T-cell lymphoma; Desmoplastic small round cell tumor; Endometrial cancer; Ependymoma; Esophageal cancer; Ewing's sarcoma in the Ewing family of tumors; Extracranial germ cell tumor, Childhood; Extragonadal Germ cell tumor; Extrahepatic bile duct cancer; Eye Cancer, Intraocular melanoma; Eye Cancer, Retinoblastoma; Gallbladder cancer; Gastric (Stomach) cancer; Gastrointestinal Carcinoid Tumor; Gastrointestinal stromal tumor (GIST); Germ cell tumor: extracranial, extragonadal, or ovarian; Gestational trophoblastic tumor; Glioma of the brain stem; Glioma, Childhood Cerebral Astrocytoma; Glioma, Childhood Visual Pathway and Hypothalamic; Gastric carcinoid; Hairy cell leukemia; Head and neck cancer; Heart cancer; Hepatocellular (liver) cancer; Hodgkin lymphoma; Hypopharyngeal cancer; Hypothalamic and visual pathway glioma, childhood; Intraocular Melanoma; Islet Cell Carcinoma (Endocrine Pancreas); Kaposi sarcoma; Kidney cancer (renal cell cancer); Laryngeal Cancer; Leukemias; Leukemia, acute lymphoblastic (also called acute lymphocytic leukemia); Leukemia, acute myeloid (also called acute myelogenous leukemia); Leukemia, chronic lymphocytic (also called chronic lymphocytic leukemia); Leukemia, chronic myelogenous (also called chronic myeloid leukemia); Leukemia, hairy cell; Lip and Oral Cavity Cancer; Liver Cancer (Primary); Lung Cancer, Non-Small Cell; Lung Cancer, Small Cell; Lymphomas; Lymphoma, AIDS-related; Lymphoma, Burkitt; Lymphoma, cutaneous T-Cell; Lymphoma, Hodgkin; Lymphomas, Non-Hodgkin (an old classification of all lymphomas except Hodgkin's); Lymphoma, Primary Central Nervous System; Marcus Whittle, Deadly Disease; Macroglobulinemia, Waldenstrim; Malignant Fibrous Histiocytoma of Bone/Osteosarcoma; Medulloblastoma, Childhood; Melanoma; Melanoma, Intraocular (Eye); Merkel Cell Carcinoma; Mesothelioma, Adult Malignant; Mesothelioma, Childhood; Metastatic Squamous Neck Cancer with Occult Primary; Mouth Cancer; Multiple Endocrine Neoplasia Syndrome, Childhood; Multiple Myeloma/Plasma Cell Neoplasm; Mycosis Fungoides; Myelodysplastic Syndromes; Myelodysplastic/Myeloproliferative Diseases; Myelogenous Leukemia, Chronic; Myeloid Leukemia, Adult Acute; Myeloid Leukemia, Childhood Acute; Myeloma, Multiple (Cancer of the Bone-Marrow); Myeloproliferative Disorders, Chronic; Nasal cavity and paranasal sinus cancer; Nasopharyngeal carcinoma; Neuroblastoma; Non-Hodgkin lymphoma; Non-small cell lung cancer; Oral Cancer; Oropharyngeal cancer; Osteosarcoma/malignant fibrous histiocytoma of bone; Ovarian cancer; Ovarian epithelial cancer (Surface epithelial-stromal tumor); Ovarian germ cell tumor; Ovarian low malignant potential tumor; Pancreatic cancer; Pancreatic cancer, islet cell; Paranasal sinus and nasal cavity cancer; Parathyroid cancer; Penile cancer; Pharyngeal cancer; Pheochromocytoma; Pineal astrocytoma; Pineal germinoma; Pineoblastoma and supratentorial primitive neuroectodermal tumors, childhood; Pituitary adenoma; Plasma cell neoplasia/Multiple myeloma; Pleuropulmonary blastoma; Primary central nervous system lymphoma; Prostate cancer; Rectal cancer; Renal cell carcinoma (kidney cancer); Renal pelvis and ureter, transitional cell cancer; Retinoblastoma; Rhabdomyosarcoma, childhood; Salivary gland cancer; Sarcoma, Ewing family of tumors; Sarcoma, Kaposi; Sarcoma, soft tissue; Sarcoma, uterine; Sezary syndrome; Skin cancer (nonmelanoma); Skin cancer (melanoma); Skin carcinoma, Merkel cell; Small cell lung cancer; Small intestine cancer; Soft tissue sarcoma; Squamous cell carcinoma—see Skin cancer (nonmelanoma); Squamous neck cancer with occult primary, metastatic; Stomach cancer; Supratentorial primitive neuroectodermal tumor, childhood; T-Cell lymphoma, cutaneous (Mycosis Fungoides and Sezary syndrome); Testicular cancer; Throat cancer; Thymoma, childhood; Thymoma and Thymic carcinoma; Thyroid cancer; Thyroid cancer, childhood; Transitional cell cancer of the renal pelvis and ureter; Trophoblastic tumor, gestational; Unknown primary site, carcinoma of, adult; Unknown primary site, cancer of, childhood; Ureter and renal pelvis, transitional cell cancer; Urethral cancer; Uterine cancer, endometrial; Uterine sarcoma; Vaginal cancer; Visual pathway and hypothalamic glioma, childhood; Vulvar cancer; Waldenstrom macroglobulinemia; and Wilms tumor (kidney cancer).

In some embodiments, the host self-antigen is derived from the variable region of a T cell receptor (TCR) or an antigen derived from the variable region of a B cell receptor.

In some embodiments, the antigen may be one suitable for use in vaccine or immunological compositions (e.g. Stedman's Medical Dictionary (24th edition, 1982, e.g., definition of vaccine (for a list of antigens used in vaccine formulations); such antigens or epitopes of interest from those antigens may be used. One skilled in the art may select an antigen and the coding DNA therefor from the knowledge of the amino acid and corresponding DNA sequences of the peptide or polypeptide, as well as from the nature of particular amino acids (e.g., size, charge, etc.) and the codon dictionary.

One method to determine T epitopes of an antigen involves epitope mapping. Overlapping peptides of the tumor antigen are generated by oligo-peptide synthesis. The individual peptides are then tested for their ability to induce T cell activation. This approach has been particularly useful in mapping T cell epitopes since the T cell recognizes short linear peptides complexed with MHC molecules.

### Cmv Vectors

Disclosed herein are recombinant CMV vectors comprising a nucleic acid sequence encoding a heterologous antigen.

In some embodiments, the recombinant CMV vector is or is derived from HCMV TR3. As referred to herein, “HCMV TR3” or “TR3” refers to a HCMV-TR3 vector backbone derived from the clinical isolate HCMV TR, as described in Caposio, P et al. (Characterization of a live attenuated HCMV-based vaccine platform. Scientific Reports 9, 19236 (2019)).

As described herein, recombinant CMV vectors may be characterized by the presence or absence of one or more CMV genes. CMV vectors may also be characterized by the presence or absence of one or more proteins encoded by one or more CMV genes. A protein encoded by a CMV gene may be absent due to the presence of a mutation in the nucleic acid sequence encoding the CMV gene. In some embodiments, the vector can include an ortholog or homolog of a CMV gene. Examples of CMV genes include, but are not limited to, UL82, UL128, UL130, UL146, UL147, UL18, and UL78.

The human cytomegalovirus UL82 gene encodes pp71, a protein that is localized in the tegument domain of the virus particle. For example, the UL82 gene of the CMV TR strain is 118811 to 120490 for GenBank Accession No. KF021605.1.

Pp71 may perform one or more functions, including inhibition of Daxx repression of viral gene transcription, negative regulation of STING, and evasion of cell antiviral responses (Kalejta R F, et al. Expanding the Known Functional Repertoire of the Human Cytomegalovirus pp71 Protein. Front Cell Infect Microbiol. 2020 Mar. 12; 10:95). Deletion of UL82 or disruption of UL82 by insertion of a foreign gene at the UL82 locus results in the absence of pp71 protein and consequently reduces replication in fibroblasts, endothelial cells, epithelial cells, and astrocytes (Caposio P et al., Characterization of a live-attenuated HCMV-based vaccine platform. Sci Rep. 2019 December 17; 9(1): 19236). The effects of UL82 deletion or disruption are reversible by cell kinase inhibitors. The rhesus cytomegalovirus (RhCMV) gene RhCMV 110 is homologous to human CMV UL82 (Hansen S G, et al. Complete sequence and genomic analysis of rhesus cytomegalovirus. J Virol. 2003 June; 77(12): 6620-36).

The human cytomegalovirus genes UL128 and UL130 encode structural components of the viral envelope (Patrone, M et al. Human cytomegalovirus UL130 protein promotes endothelial cell infection through a producer cell modification of the virion. J Virol. 79(13): 8361-73 (2005); Ryckman, B J et al. Characterization of the human cytomegalovirus gH/gL/UL128-131 complex that mediates entry into epithelial and endothelial cells. J Virol. 82(1): 60-70 (2008); Wang, D et al. Human cytomegalovirus virion protein complex required for epithelial and endothelial cell tropism. Proc Natl Acad Sci USA. 102(50): 18153-8 (2005)). For example, the UL128 gene of the CMV TR strain is 176206 to 176964 for GenBank Accession No. KF021605.1 and the UL 130 gene of the CMV TR strain is 177004 to 177648 for GenBank Accession No. KF021605.1.

The human cytomegalovirus genes UL146 and UL147 encode the CXC chemokines vCXC-1 and vCXC-2, respectively (Penfold, M E et al. Cytomegalovirus encodes a potent alpha chemokine. Proc Natl Acad Sci USA. 96(17): 9839-44 (1999)). For example, the UL146 gene of the CMV TR strain is 180954 to 181307 for GenBank Accession No. KF021605.1 and the UL 147 gene of the CMV TR strain is 180410 to 180889 for GenBank Accession No. KF021605.1.

The human cytomegalovirus UL18 gene encodes a type-I membrane glycoprotein that associates with β2-microglobulin and can bind endogenous peptides (Park, B et al. Human cytomegalovirus inhibits tapasin-dependent peptide loading and optimization of the MHC class I peptide cargo for immune evasion. Immunity. 20(1): 71-85 (2004); Browne, H et al. A complex between the MHC class I homologue encoded by human cytomegalovirus and beta 2 microglobulin. Nature. 347(6295): 770-2 (1990); Fahnestock, M L et al. The MHC class I homolog encoded by human cytomegalovirus binds endogenous peptides. Immunity. 3(5): 583-90 (1995)). For example, the UL18 gene of the CMV TR strain is 24005 to 25111 for GenBank Accession No. KF021605.1.

The human cytomegalovirus UL78 gene encodes a putative G protein-coupled receptor (Chee, M S et al. Analysis of the protein-coding content of the sequence of human cytomegalovirus strain AD169. Curr Top Microbiol Immunol. 1154:125-69 (1990)) and may also have a role in viral replication (Michel, D et al. The human cytomegalovirus UL78 gene is highly conserved among clinical isolates, but is dispensable for replication in fibroblasts and a renal artery organ-culture system. J Gen Virol. 86 (Pt 2): 297-306 (2005)). For example, the UL78 gene of the CMV TR strain is 114247 to 115542 for GenBank Accession No. KF021605.1.

In some embodiments, the recombinant CMV vector (e.g., a recombinant HCMV vector comprising a TR3 backbone) does not express UL128, UL130, UL146, or UL147, or orthologs thereof, due to the presence of a mutation in the nucleic acid sequences encoding UL128, UL130, UL146, or UL147, or the ortholog thereof. In some embodiments, the CMV vector is also deficient for one or more of UL18, UL78, and UL82, and orthologs thereof, due to the presence of a mutation in the nucleic acid sequence encoding UL18, UL78, or UL82, or the ortholog thereof. In some embodiments, the CMV vector is also deficient for US11, and orthologs thereof, due to the presence of a mutation in the nucleic acid sequence encoding US11, or the ortholog thereof. In the aforementioned embodiments, the mutation or mutations may be any mutation that results in a lack of expression of active proteins. Such mutations include, for example, point mutations, frameshift mutations, deletions of less than all of the sequence that encodes the protein (truncation mutations), or deletions of all of the nucleic acid sequence that encodes the protein. In some embodiments, the recombinant CMV vector (e.g., a recombinant HCMV vector comprising a TR3 backbone) also expresses UL40 and US28, or orthologs thereof.

In some embodiments, the recombinant CMV vector (e.g., a recombinant HCMV vector comprising a TR3 backbone) does not express UL82, UL128, UL130, UL146, or UL147, or orthologs thereof, due to the presence of a mutation in the nucleic acid sequences encoding UL82, UL128, UL130, UL146, and UL147, or orthologs thereof. In some embodiments, the recombinant CMV vector is also deficient for UL18, due to the presence of a mutation in the nucleic acid sequence encoding UL18.

In some embodiments, the recombinant CMV vector (e.g., a recombinant HCMV vector comprising a TR3 backbone) does not express UL78, UL128, UL130, UL146, or UL147, or orthologs thereof, due to the presence of a mutation in the nucleic acid sequences encoding UL78, UL128, UL130, UL146, and UL147, or orthologs thereof. In some embodiments, the recombinant CMV vector is also deficient for UL18, due to the presence of a mutation in the nucleic acid sequence encoding UL18.

In some embodiments, the recombinant CMV vector (e.g., a recombinant HCMV vector comprising a TR3 backbone) does not express UL78, UL82, UL128, UL130, UL146, or UL147, or orthologs thereof, due to the presence of a mutation in the nucleic acid sequences encoding UL78, UL82, UL128, UL130, UL146, and UL147, or orthologs thereof.

A challenge for manufacturing HCMV vectors having desirable properties for vaccines is that the vectors are often designed to have reduced viral replication or growth. For example, some live attenuated HCMV-HIV vaccine vectors are engineered to be growth deficient by deletion of the HCMV gene UL82 (which encodes the tegument protein pp71), resulting in lower viral yield. pp71 is important for wild type HCMV infection because this tegument protein is translocated to the nucleus where it suppresses cellular Daxx function, thus allowing CMV immediate-early (IE) gene expression that triggers the replication cycle. Some manufacturing processes rely on functional complementation using transient transfection of MRC-5 cells with an siRNA targeting Daxx, which mimics one of the functions of HCMV pp71. Another approach is to use transfection of a mRNA encoding pp71, to enable the host cell to express the essential viral gene. Transfection of a mRNA for expressing the essential viral gene may be able to provide all of the functions of the gene that are likely to enhance the infection process, such as cell cycle stimulation, efficient virion packaging, and virus stability. In addition, protein present late in infection has the potential to be packaged in the progeny virus, which could lower the required dose of the vaccine by more efficient first round infection and establishment of persistent infection. Accordingly, in some embodiments, the present disclosure provides a method of producing a recombinant CMV viral vector, comprising: (a) introducing a mRNA encoding a pp71 protein to a cell; (b) infecting the cell with a recombinant CMV; (c) incubating the cell; and (d) collecting the recombinant CMV viral vector. In some embodiments, the nucleic acid encoding a pp71 protein is delivered to the cell using transfection. In some embodiments, the cell is a MRC-5 cell. In some embodiments, the recombinant CMV is a recombinant HCMV as described herein (e.g., a recombinant HCMV vector derived from a TR3 backbone). In some embodiments, the recombinant CMV and recombinant CMV viral vector comprises a nucleic acid encoding a heterologous pathogen-specific antigen, such as a human immunodeficiency virus (HIV) antigen as described herein. A CMV viral vector made by such a method is also within the scope of the disclosure.

In some embodiments, the recombinant CMV vector (e.g., a recombinant HCMV vector comprising a TR3 backbone) comprises a nucleic acid sequence encoding a microRNA (miRNA) recognition element (MRE). In some embodiments, the HCMV vector comprises a nucleic acid sequence encoding an MRE that contains target sites for microRNAs expressed in endothelial cells. Examples of miRNAs expressed in endothelial cells are miR 126, miR-126-3p, miR-130a, miR-210, miR-221/222, miR-378, miR-296, and miR-328. In some embodiments, the HCMV vector lacks UL18, UL128, UL130, UL146, and UL147 (and optionally UL82) and expresses UL40 and US28 and the MRE contains target sites for microRNAs expressed in endothelial cells.

In some embodiments, the recombinant CMV vector (e.g., a recombinant HCMV vector comprising a TR3 backbone) comprises a nucleic acid sequence encoding an MRE that contains target sites for microRNAs expressed in myeloid cells. Examples of miRNAs expressed in myeloid cells are miR-142-3p, miR-223, miR-27a, miR-652, miR-155, miR-146a, miR-132, miR-21, miR-124, and miR-125.

MREs that may be included in the recombinant CMV vector disclosed herein may be any miRNA recognition element that silences expression in the presence of a miRNA expressed by endothelial cells, or any miRNA recognition element that silences expression in the presence of a miRNA expressed by myeloid cells. Such an MRE may be the exact complement of a miRNA. Alternatively, other sequences may be used as MREs for a given miRNA. For example, MREs may be predicted from sequences using publicly available data bases. In one example, the miRNA may be searched on the website microRNA.org (www.microrna.org). In turn, a list of mRNA targets of the miRNA will be listed. For each listed target on the page, ‘alignment details’ may be accessed and putative MREs accessed. One of ordinary skill in the art may select a validated, putative, or mutated MRE sequence from the literature that would be predicted to induce silencing in the presence of a miRNA expressed in a myeloid cell such as a macrophage. One example involves the above referenced website. The person of ordinary skill in the art may then obtain an expression construct whereby a reporter gene (such as a fluorescent protein, enzyme, or other reporter gene) has expression driven by a promoter such as a constitutively active promoter or cell specific promoter. The MRE sequence may then be introduced into the expression construct. The expression construct may be transfected into an appropriate cell, and the cell transfected with the miRNA of interest. A lack of expression of the reporter gene indicates that the MRE silences gene expression in the presence of the miRNA.

In some embodiments, the CMV vector comprises a nucleic acid sequence that does not encode any MREs.

In some embodiments, the CMV vectors described herein contain mutations that may prevent host to host spread, thereby rendering the virus unable to infect immunocompromised or other subjects that could face complications as a result of CMV infection. The CMV vectors described herein may also contain mutations that result in the presentation of immunodominant and nonimmunodominant epitopes as well as non-canonical MHC restriction. However, in some embodiments, mutations in the CMV vectors described herein do not affect the ability of the vector to reinfect a subject that has been previously infected with CMV. Such CMV mutations are described in, for example, U.S. Patent Application Publication Nos. US2013/0136768A1, US2013/0142823A1; US2014/0141038A1; and International Application Publication No. WO2014/138209A1, which mutations are incorporated by reference herein.

In some embodiments, the heterologous antigen may be a pathogen-specific antigen, a tumor antigen, a tumor-specific antigen, or a host self-antigen, as described above.

In some embodiments, the present disclosure provides a recombinant HCMV vector comprising a nucleic acid sequence having at least 90%, 91%, 92%, 93%, 94%, 95%, 96%, 97%, 98%, 99%, or 100% identity to the nucleic acid sequence according to SEQ ID NO:7. In some embodiments, the recombinant HCMV vector comprises the nucleic acid sequence according to SEQ ID NO:7. In some embodiments, the recombinant HCMV vector consists of the nucleic acid sequence according to SEQ ID NO: 7.

In some embodiments, the present disclosure provides a recombinant HCMV vector comprising a nucleic acid sequence having at least 90%, 91%, 92%, 93%, 94%, 95%, 96%, 97%, 98%, 99%, or 100% identity to the nucleic acid sequence according to SEQ ID NO:9. In some embodiments, the recombinant HCMV vector comprises the nucleic acid sequence according to SEQ ID NO:9. In some embodiments, the recombinant HCMV vector consists of the nucleic acid sequence according to SEQ ID NO: 9.

In some embodiments, the present disclosure provides a recombinant CMV vector comprising a nucleic acid sequence having at least 90%, at least 91%, at least 92%, at least 93%, at least 94%, at least 95%, at least 96%, at least 97%, at least 98%, at least 99%, or at least 100% identity to the nucleic acid sequence according to SEQ ID NO: 5. In some embodiments, the recombinant HCMV vector comprises the nucleic acid sequence according to SEQ ID NO:5. In some embodiments the recombinant HCMV vector consists of the nucleic acid sequence according to SEQ ID NO:5.

In some embodiments, the present disclosure provides a recombinant HCMV vector comprising a nucleic acid sequence having at least 90%, at least 91%, at least 92%, at least 93%, at least 94%, at least 95%, at least 96%, at least 97%, at least 98%, at least 99%, or at least 100% identity to the nucleic acid sequence according to SEQ ID NO: 6. In some embodiments, the recombinant HCMV vector comprises the nucleic acid sequence according to SEQ ID NO:6. In some embodiments, the recombinant HCMV vector consists of the nucleic acid sequence according to SEQ ID NO:6.

In some embodiments, the present disclosure provides a recombinant HCMV vector comprising a nucleic acid sequence having at least 90%, at least 91%, at least 92%, at least 93%, at least 94%, at least 95%, at least 96%, at least 97%, at least 98%, at least 99%, or at least 100% identity to the nucleic acid sequence according to SEQ ID NO: 8. In some embodiments, the recombinant HCMV vector comprises the nucleic acid sequence according to SEQ ID NO:8. In some embodiments, the recombinant HCMV vector consists of the nucleic acid sequence according to SEQ ID NO:8.

The CMV vectors disclosed herein may be prepared by inserting DNA comprising a sequence that encodes the heterologous antigen into an essential or non-essential region of the CMV genome. In some embodiments, the heterologous antigen replaces all or part of UL78 or UL82. In some embodiments, the heterologous antigen replaces all or part of UL78 and is operably linked to the UL78 promoter. In some embodiments, the heterologous antigen replaces all or part of UL82 and is operably linked to the UL82 promoter. The method may further comprise deleting one or more regions from the CMV genome. The method may comprise in vivo recombination. Thus, the method may comprise transfecting a cell with CMV DNA in a cell-compatible medium in the presence of donor DNA comprising the heterologous DNA flanked by DNA sequences homologous with portions of the CMV genome, whereby the heterologous DNA is introduced into the genome of the CMV, and optionally then recovering CMV modified by the in vivo recombination. The method may also comprise cleaving CMV DNA to obtain cleaved CMV DNA, ligating the heterologous DNA to the cleaved CMV DNA to obtain hybrid CMV-heterologous DNA, transfecting a cell with the hybrid CMV-heterologous DNA, and optionally then recovering CMV modified by the presence of the heterologous DNA Since in vivo recombination is comprehended, the method accordingly also provides a plasmid comprising donor DNA not naturally occurring in CMV encoding a polypeptide foreign to CMV, the donor DNA is within a segment of CMV DNA that would otherwise be co-linear with an essential or non-essential region of the CMV genome such that DNA from an essential or nonessential region of CMV is flanking the donor DNA The heterologous DNA may be inserted into CMV to generate the recombinant CMV in any orientation that yields stable integration of that DNA, and expression thereof, when desired.

The DNA encoding the heterologous antigen in the recombinant CMV vector may also include a promoter. The promoter may be from any source such as a herpes virus, including an endogenous cytomegalovirus (CMV) promoter, such as a human CMV (HCMV), rhesus macaque CMV (RhCMV), murine, or other CMV promoter. The promoter may also be a nonviral promoter such as the EF1α promoter. The promoter may be a truncated transcriptionally active promoter which comprises a region transactivated with a transactivating protein provided by the virus and the minimal promoter region of the full-length promoter from which the truncated transcriptionally active promoter is derived. The promoter may be composed of an association of DNA sequences corresponding to the minimal promoter and upstream regulatory sequences. A minimal promoter is composed of the CAP site plus ATA box (minimum sequences for basic level of transcription; unregulated level of transcription); “upstream regulatory sequences” are composed of the upstream element(s) and enhancer sequence(s). Further, the term “truncated” indicates that the full-length promoter is not completely present, i.e., that some portion of the full-length promoter has been removed. The truncated promoter may be derived from a herpesvirus such as MCMV or HCMV, e.g., HCMV-IE or MCMV-IE. There may be up to a 40% and even up to a 90% reduction in size, from a full-length promoter, based upon base pairs. The promoter may also be a modified non-viral promoter. As to HCMV promoters, reference is made to U.S. Pat. Nos. 5,168,062 and 5,385,839. As to transfecting cells with plasmid DNA for expression therefrom, reference is made to Felgner, J H et al. (Enhanced gene delivery and mechanism studies with a novel series of cationic lipid formulations. J Biol. Chem. 269, 2550-2561 (1994)). As to direct injection of plasmid DNA as a simple and effective method of vaccination against a variety of infectious diseases reference is made to Ulmer, J B et al. (Heterologous protection against influenza by injection of DNA encoding a viral protein. Science 259, 1745-1749 (1993)). It is therefore within the scope of this disclosure that the vector may be used by the direct injection of vector DNA. Also disclosed is an expression cassette that may be inserted into a recombinant virus or plasmid comprising a truncated transcriptionally active promoter. The expression cassette may further include a functional truncated polyadenylation signal; for instance an SV40 polyadenylation signal which is truncated, yet functional. A truncated polyadenylation signal addresses the insert size limit problems of recombinant viruses such as CMV. The expression cassette may also include heterologous DNA with respect to the virus or system into which it is inserted; and that DNA may be heterologous DNA as described herein.

It is noted that the DNA comprising the sequence encoding the heterologous antigen may itself include a promoter for driving expression in the CMV vector or the DNA may be limited to the coding DNA of the antigen. This construct may be placed in such an orientation relative to an endogenous CMV promoter that it is operably linked to the promoter and is thereby expressed. Further, multiple copies of DNA encoding the antigen or use of a strong or early promoter or early and late promoter, or any combination thereof, may be done so as to amplify or increase expression. Thus, the DNA encoding the antigen may be suitably positioned with respect to a CMV endogenous promoter, or those promoters may be translocated to be inserted at another location together with the DNA encoding the antigen. Nucleic acids encoding more than one antigen may be packaged in the CMV vector.

### Pharmaceutical Compositions

The recombinant CMV vectors disclosed herein may be used in a pharmaceutical composition (e.g., an immunogenic or vaccine composition) containing the vector and a pharmaceutically acceptable carrier or diluent. An immunogenic or vaccine composition containing the recombinant CMV virus or vector (or an expression product thereof) elicits an immunological response (local or systemic). The response can, but need not be, protective. In other words, an immunogenic or vaccine composition elicits a local or systemic protective or therapeutic response.

Such pharmaceutical compositions may be prepared in accordance with standard techniques well known to those skilled in the pharmaceutical arts. Such compositions may be administered in dosages and by techniques well known to those skilled in the medical arts taking into consideration such factors as the breed or species, age, sex, weight, and condition of the particular patient, and the route of administration. The compositions may be administered alone, or may be co-administered or sequentially administered with other CMV vectors or with other immunological, antigenic or vaccine or therapeutic compositions. Such other compositions may include purified native antigens or epitopes or antigens or epitopes from the expression by a recombinant CMV or another vector system.

Pharmaceutical compositions as disclosed herein may be formulated so as to be used in any administration procedure known in the art. Such pharmaceutical compositions may be via a parenteral route (intradermal, intraperitoneal, intramuscular, subcutaneous, intravenous, or others). The administration may also be via a mucosal route, e.g., oral, nasal, genital, etc.

Examples of compositions include liquid preparations for orifice, e.g., oral, nasal, anal, genital, e.g., vaginal, etc., administration such as suspensions, syrups or elixirs; and, preparations for parenteral, subcutaneous, intraperitoneal, intradermal, intramuscular or intravenous administration (e.g., injectable administration) such as sterile suspensions or emulsions. In such compositions the recombinant may be in admixture with a suitable carrier, diluent, or excipient such as sterile water, physiological saline, glucose, trehalose, or the like.

Pharmaceutical compositions disclosed herein typically may contain an adjuvant and an amount of the CMV vector or expression product to elicit the desired response. In human applications, alum (aluminum phosphate or aluminum hydroxide) is a typical adjuvant. Saponin and its purified component Quil A, Freund's complete adjuvant and other adjuvants used in research and veterinary applications have toxicities which limit their potential use in human vaccines. Chemically defined preparations such as muramyl dipeptide, monophosphoryllipid A, phospholipid conjugates such as those described by Goodman-Snitkoff, G. et al. (Role of intrastructural/intermolecular help in immunization with peptide-phospholipid complexes. J Immunol. 147, 410-415 (1991)), encapsulation of the protein within a proteoliposome as described by Miller, M D et al. (Vaccination of rhesus monkeys with synthetic peptide in a fusogenic proteoliposome elicits simian immunodeficiency virus-specific CD8+ cytotoxic T lymphocytes. J Exp. Med. 176, 1739-1744 (1992)), and encapsulation of the protein in lipid vesicles such as Novasome lipid vesicles (Micro Vescular Systems, Inc., Nashua, N.H.) may also be used.

The composition may be packaged in a single dosage form for immunization by parenteral (e.g., intramuscular, intradermal or subcutaneous) administration or orifice administration, e.g., perlingual (e.g., oral), intragastric, mucosal including intraoral, intraanal, intravaginal, and the like administration. The effective dosage and route of administration are determined by the nature of the composition, by the nature of the expression product, by expression level if recombinant CMV is directly used, and by known factors, such as breed or species, age, sex, weight, condition, and nature of the subject, as well as LD50 and other screening procedures which are known and do not require undue experimentation. Dosages of expressed product may range from a few to a few hundred micrograms, e.g., 5 to 500 μg. The CMV vector may be administered in any suitable amount to achieve expression at these dosage levels. In nonlimiting examples: CMV vectors may be administered in an amount of at least 10₂ pfu; thus, CMV vectors may be administered in at least this amount; or in a range from about 10₂ pfu to about 10₇ pfu. In nonlimiting examples: CMV vectors may be administered in an amount of at least 1×10³ focus-forming units (ffu); thus, CMV vectors may be administered in at least this amount; or in a range from about 1×10³ to about 1×10⁷ ffu.

In nonlimiting examples: the CMV vectors may be administered in an amount of about 1×10³ ffu, about 3×10⁴ ffu, about 5×10⁴ ffu, about 5×10⁵ ffu, about 1×10⁶ ffu, about 5×10⁶ ffu, or about 1×10⁷ ffu. In nonlimiting examples: the CMV vectors may be administered in one dose, at least one dose, two doses, or at least two doses. As a non-limiting example, the CMV vectors may be administered in two doses. An initial dose may be referred to as a “prime” dose and any subsequent dose or doses may be referred to as a “boost” dose or “boost” doses. As a non-limiting example, a “boost” dose may be administered at about 84 days, or 12 weeks after administration of the “prime” dose. Other suitable carriers or diluents may be water or a buffered saline, with or without a preservative. The CMV vector may be lyophilized for resuspension at the time of administration or may be in solution. In nonlimiting examples: the suspended CMV vector may be administered as an injection having a volume of less than 1 ml, about 1 ml, about 2 ml, or more than 1 ml. In a nonlimiting example, the CMV vector may be administered subcutaneously, optionally, in the deltoid region.

### Methods of Treatment and Other Uses

The antigens and recombinant CMV vectors disclosed herein may be used in methods of inducing an immunological or immune response in a subject comprising administering to the subject a composition comprising the recombinant CMV virus or vector and a pharmaceutically acceptable carrier or diluent.

As used herein, the term “subject” refers to a living multi-cellular vertebrate organisms, a category that includes both human and non-human mammals. The subject may be an animal, such as a mammal, including any mammal that can be infected with HIV, e.g., a primate (such as a human, a non-human primate, e.g., a monkey, or a chimpanzee), or an animal that is considered an acceptable clinical model of pathogenic infection, such as the HBV-AAV mouse model (see, e.g., Yang, D Y et al. A mouse model for HBV immunotolerance and immunotherapy. Cell and Mol Immunol 11, 71-78 (2014)) or the HBV 1.3×fs transgenic mouse model (Guidotti, L G et al. High-level hepatitis B virus replication in transgenic mice. J. Virol. 69, 6158-6169 (1995)).

In some embodiments, the subject is human.

In some embodiments, the subject has a serological status with regard to HCMV infection. As used herein, the term “seropositive” refers to a subject or immune system that has been previously exposed to a particular antigen and thus has a detectable serum antibody titer against the antigen of interest. The phrase “seropositive for HCMV” refers to a subject or immune system that has been previously exposed to a HCMV antigen. A seropositive subject or immune system can be distinguished by the presence of antibodies or other immune markers in the serum that indicate past exposure to a particular antigen. As used herein, the term “seronegative” refers to a subject or immune system that has not been previously exposed to a particular antigen and thus has an absence of detectable serum antibody titer against the antigen of interest. The phrase “seronegative for HCMV” refers to a subject or immune system that has not been previously exposed to a HCMV antigen.

As used herein, the term “treatment” refers to an intervention that ameliorates a sign or symptom of a disease or pathological condition. As used herein, the terms “treatment”, “treat”, and “treating,” with reference to a disease, pathological condition or symptom, also refers to any observable beneficial effect of the treatment. The beneficial effect may be evidenced, for example, by a delayed onset of clinical symptoms of the disease in a susceptible subject, a reduction in severity of some or all clinical symptoms of the disease, a slower progression of the disease, a reduction in the number of relapses of the disease, an improvement in the overall health or well-being of the subject, or by other parameters well known in the art that are specific to the particular disease. A prophylactic treatment is a treatment administered to a subject who does not exhibit signs of a disease or exhibits only early signs, for the purpose of decreasing the risk of developing pathology. A therapeutic treatment is a treatment administered to a subject after signs and symptoms of the disease have developed.

As used herein, the terms “preventing” or “prevention” refer to the failure to develop a disease, disorder, or condition, or the reduction in the development of a sign or symptom associated with such a disease, disorder, or condition (e.g., by a clinically relevant amount), or the exhibition of delayed signs or symptoms delayed (e.g., by days, weeks, months, or years). Prevention may require the administration of more than one dose.

As used herein, the term “effective amount” refers to an amount of an agent, such as a CMV vector comprising a heterologous antigen, that is sufficient to generate a desired response, such as reduce or eliminate a sign or symptom of a condition or disease or induce an immune response to an antigen. In some examples, an “effective amount” is one that treats (including prophylaxis) one or more symptoms and/or underlying causes of any of a disorder or disease. An effective amount may be a therapeutically effective amount, including an amount that prevents one or more signs or symptoms of a particular disease or condition from developing, such as one or more signs or symptoms associated with infectious disease or cancer.

The disclosed CMV vectors may be administered in vivo, for example where the aim is to produce an immunogenic response, including a CD8+ T cell/immune response, including an immune response characterized by a high percentage of the CD8+ T cell response being restricted by MHC-E, MHC-II, or MHC-I (or a homolog or ortholog thereof). For example, in some examples it may be desired to use the disclosed CMV vectors in a laboratory animal, such as rhesus macaques for preclinical testing of immunogenic compositions and vaccines using RhCMV. In other examples, it will be desirable to use the disclosed CMV vectors in human subjects, such as in clinical trials and for actual clinical use of the immunogenic compositions using HCMV.

For such in vivo applications the disclosed CMV vectors may be administered as a component of an immunogenic or pharmaceutical composition further comprising a pharmaceutically acceptable carrier. In some embodiments, the immunogenic compositions of the disclosure are useful to stimulate an immune response against the heterologous antigen, and may be used as one or more components of a prophylactic or therapeutic vaccine. The nucleic acids and vectors of the disclosure are particularly useful for providing genetic vaccines, i.e., vaccines for delivering the nucleic acids encoding the antigens of the disclosure to a subject, such as a human, such that the antigens are then expressed in the subject to elicit an immune response.

Immunization schedules (or regimens) are well known for animals (including humans) and may be readily determined for the particular subject and immunogenic composition. Hence, the immunogens may be administered one or more times to the subject. Preferably, there is a set time interval between separate administrations of the immunogenic composition. While this interval varies for every subject, typically it ranges from 10 days to several weeks, and is often 2, 4, 6, 8, or 12 weeks. For humans, the interval is typically from 2 to 6 weeks. In a particularly advantageous embodiment of the present disclosure, the interval is longer, advantageously about 10 weeks, 12 weeks, 14 weeks, 16 weeks, 18 weeks, 20 weeks, 22 weeks, 24 weeks, 26 weeks, 28 weeks, 30 weeks, 32 weeks, 34 weeks, 36 weeks, 38 weeks, 40 weeks, 42 weeks, 44 weeks, 46 weeks, 48 weeks, 50 weeks, 52 weeks, 54 weeks, 56 weeks, 58 weeks, 60 weeks, 62 weeks, 64 weeks, 66 weeks, 68 weeks, or 70 weeks. The immunization regimes typically have from 1 to 6 administrations of the immunogenic composition, but may have as few as one or two or four. The methods of inducing an immune response may also include administration of an adjuvant with the immunogens. In some instances, annual, biannual, or other long interval (5-10 years) booster immunization may supplement the initial immunization protocol. The present methods also include a variety of prime-boost regimens. In these methods, one or more priming immunizations are followed by one or more boosting immunizations. The actual immunogenic composition may be the same or different for each immunization and the type of immunogenic composition (e.g., containing protein or expression vector), the route, and formulation of the immunogens may also be varied. For example, if an expression vector is used for the priming and boosting steps, it may either be of the same or different type (e.g., DNA or bacterial or viral expression vector). One useful prime-boost regimen provides for two priming immunizations, four weeks apart, followed by two boosting immunizations at 4 and 8 weeks after the last priming immunization. It should also be readily apparent to one of skill in the art that there are several permutations and combinations that are encompassed using the DNA, bacterial, and viral expression vectors of the disclosure to provide priming and boosting regimens. CMV vectors may be used repeatedly while expressing different antigens derived from different pathogens.

Accordingly, the present disclosure provides in some embodiments a method of generating an immune response in a subject, comprising administering to the subject any of the aforementioned recombinant HCMV vectors or compositions comprising the same. In some embodiments, the immune response is to the at least one heterologous antigen delivered by the vector. In some embodiments, the recombinant HCMV vector is administered in an amount effective to elicit a CD8+ T cell response to the at least one heterologous antigen.

Also within the scope of the disclosure is the use of any of the aforementioned recombinant HCMV vectors or compositions comprising the same in the manufacture of a medicament for use in generating an immune response in a subject. The present disclosure also provides recombinant HCMV vectors and related compositions for use in generating an immune response in a subject.

In some embodiments, the disclosure provides a method of preventing a disease in a subject, comprising administering a recombinant HCMV vector or composition as disclosed herein in an amount effective to elicit a CD8+ T cell response to the at least one heterologous antigen. In some embodiments, the present disclosure provides for the use of a recombinant HCMV vector or composition disclosed herein in the manufacture of a medicament for use in preventing a disease in a subject. The present disclosure also provides recombinant HCMV vectors and related compositions for use in preventing a disease in a subject.

In further embodiments, the disclosure provides a method of preventing a disease in a subject or comprising administering a recombinant HCMV vector or composition as disclosed herein in an amount effective to: (i) elicit a CD8+ T cell response to at least one HIV antigen; (ii) reduce viremia and/or detectable HIV load, including reducing detectable HIV load below the limit of detection by any suitable test (e.g. polymerase chain reaction (PCR)); (iii) contain HIV replication and/or mutation such that primary HIV infection is rapidly aborted; and, (iv) avert sustained infection and disease such that life-long antiviral treatment (ART) is not required. In some embodiments, the present disclosure provides for the use of a recombinant HCMV vector or composition disclosed herein in the manufacture of a medicament for use in preventing a disease in a subject. The present disclosure also provides recombinant HCMV vectors and related compositions for use in preventing a disease in a subject.

In some embodiments, the disclosure provides a method of treating a disease in a subject or comprising administering a recombinant HCMV vector or composition as disclosed herein in an amount effective to elicit a CD8+ T cell response to the at least one heterologous antigen. In some embodiments, the present disclosure provides for the use of a recombinant HCMV vector or composition disclosed herein in the manufacture of a medicament for use in treating a disease in a subject. The present disclosure also provides recombinant HCMV vectors and related compositions for use in treating a disease in a subject.

In further embodiments, the disclosure provides a method of treating a disease in a subject or comprising administering a recombinant HCMV vector or composition as disclosed herein in an amount effective to: (i) treat a subject having an HIV infection (ii) elicit a CD8+ T cell response to at least one HIV antigen; (iii) reduce viremia and/or detectable HIV load, including reducing detectable HIV load below the limit of detection by any suitable test (e.g. polymerase chain reaction (PCR)); (iv) contain HIV replication and/or mutation such that primary HIV infection is rapidly aborted; and, (v) avert sustained infection and disease such that life-long antiviral treatment (ART) is not required. In some embodiments, the present disclosure provides for the use of a recombinant HCMV vector or composition disclosed herein in the manufacture of a medicament for use in treating a disease in a subject. The present disclosure also provides recombinant HCMV vectors and related compositions for use in treating a disease in a subject.

In some embodiments, “sustained” HIV infection may refer to (1) detection of at least 10,000 HIV copies per milliliter of blood or (2) detection of HIV in blood samples for three or more consecutive weeks.

In some embodiments of the aforementioned methods, uses, or compositions for use, the heterologous antigen is or comprises a HIV antigen and the disease is HIV infection.

In some embodiments of the aforementioned methods, uses, or compositions for use, the heterologous antigen is a pathogen-specific antigen, a tumor antigen, a tumor-specific antigen, or a host self-antigen, and the disease is a pathogenic infection, a tumor or cancer, or an autoimmune disease.

In some embodiments of the aforementioned methods, uses, or compositions for use, at least 10% of the CD8+ T cells elicited by the recombinant HCMV vector are restricted by MHC-E or an ortholog thereof. In some further embodiments, at least 20%, at least 30%, at least 40%, at least 50%, at least 60%, at least 75%, at least 80%, at least 85%, at least 90%, or at least 95% of the CD8+ T cells elicited by the recombinant HCMV vector are restricted by MHC-E or an ortholog thereof.

In some embodiments of the aforementioned methods, uses, or compositions for use, wherein at least 10% of the CD8+ T cells elicited by the recombinant HCMV vector are restricted by MHC-II or an ortholog thereof. In some further embodiments, at least 20%, at least 30%, at least 40%, at least 50%, at least 60% or at least 75% of the CD8+ T cells elicited by the recombinant HCMV vector are restricted by MHC-II or an ortholog thereof.

In some embodiments of the aforementioned methods, uses, or compositions for use, fewer than 10%, fewer than 20%, fewer than 30%, fewer than 40%, or fewer than 50% of the CD8+ T cells elicited by the recombinant HCMV vector are restricted by MHC-class Ia or an ortholog thereof.

In some further aspects, the present disclosure provides a method of generating CD8+ T cells that recognize MHC-E/peptide complexes, by administering a recombinant CMV vector as disclosed herein. In some embodiments, the method comprises:
(a) administering to a first subject a recombinant HCMV vector as disclosed herein in an amount effective to generate a set of CD8+ T cells that recognize MHC-E/heterologous antigen-derived peptide complexes;
(b) identifying a first CD8+ TCR from the set of CD8+ T cells, wherein the first CD8+ TCR recognizes a MHC-E/peptide complex;
(c) isolating one or more CD8+ T cells from a second subject; and
(d) transfecting the one or more CD8+ T cells isolated from the second subject with an expression vector, wherein the expression vector comprises a nucleic acid sequence encoding a second CD8+ TCR and a promoter operably linked to the nucleic acid sequence encoding the second CD8+ TCR, wherein the second CD8+ TCR comprises CDR3α and CDR3β of the first CD8+ TCR, thereby generating one or more CD8+ T cells that recognize MHC-E/peptide complexes.

In some embodiments, the first subject is seropositive for HCMV. In some embodiments, the first subject is seronegative for HCMV.

In some embodiments, the present disclosure provides a method of generating CD8+ T cells that recognize MHC-E/peptide complexes, the method comprising:
(a) identifying a first CD8+ TCR from a set of CD8+ T cells, wherein the set of CD8+ T cells are isolated from a first subject that has been administered the recombinant HCMV vector of any one of claims 1-26, and wherein the first CD8+ TCR recognizes a MHC-E/heterologous antigen-derived peptide complex;
(b) isolating one or more CD8+ T cells from a second subject; and
(c) transfecting the one or more CD8+ T cells isolated from the second subject with an expression vector, wherein the expression vector comprises a nucleic acid sequence encoding a second CD8+ TCR and a promoter operably linked to the nucleic acid sequence encoding the second CD8+ TCR, wherein the second CD8+ TCR comprises CDR3a and CDR3B of the first CD8+ TCR, thereby generating one or more TCR-transgenic CD8+ T cells that recognize MHC-E/peptide complexes. In some embodiments of the method of generating CD8+ T cells that recognize MHC-E/peptide complexes, the first CD8+ TCR is identified by DNA or RNA sequencing. In some embodiments, the nucleic acid sequence encoding the second CD8+ TCR is identical to the nucleic acid sequence encoding the first CD8+ TCR. In some embodiments, the first and second subjects are human. In some embodiments, the first subject is seropositive for HCMV. In some embodiments, the first subject is seronegative for HCMV.

The present disclosure also provides a CD8+ T cell generated by the aforementioned methods. In some further embodiments, the CD8+ T cell is used in a method of treating or preventing a disease in a subject. The CD8+ T cell may be used in still further embodiments in the manufacture of a medicament for use in treating or preventing a disease in a subject.

### EXAMPLE EMBODIMENTS

In some embodiments, the present disclosure provides:
1. A recombinant HCMV vector comprising a TR3 backbone and a nucleic acid sequence encoding a heterologous antigen, wherein:
(a) (i) the vector does not express UL18, UL78, UL128, UL130, UL146, or UL147, or orthologs thereof;
   (ii) the vector comprises a nucleic acid sequence encoding UL82, or an ortholog thereof; and
   (iii) the heterologous antigen replaces all or part of UL78 and is operably linked to the UL78 promoter;
(b) (i) the vector does not express UL82, UL128, UL130, UL146, or UL147, or orthologs thereof;
   (ii) the vector comprises a nucleic acid sequence encoding UL18, or an ortholog thereof, and a nucleic acid sequence encoding UL78, or an ortholog thereof; and
   (iii) the heterologous antigen replaces all or part of UL82 and is operably linked to the UL82 promoter; or
(c) (i) the vector does not express UL18, UL82, UL128, UL130, UL146, or UL147, or orthologs thereof;
   (ii) the vector comprises a nucleic acid sequence encoding UL78, or orthologs thereof; and
   (iii) the heterologous antigen replaces all or part of UL82 and is operably linked to the UL82 promoter.
2. The recombinant HCMV vector of embodiment 1, wherein:
(i) the vector does not express UL18, UL78, UL128, UL130, UL146, or UL147;
(ii) the vector comprises a nucleic acid sequence encoding UL82, or an ortholog thereof; and
(iii) the heterologous antigen replaces all or part of UL78 and is operably linked to the UL78 promoter.
3. A recombinant HCMV vector of embodiment 1, wherein:
(i) the vector does not express UL82, UL128, UL130, UL146, or UL147, or orthologs thereof;
(ii) the vector comprises a nucleic acid sequence encoding UL18, or an ortholog thereof, and a nucleic acid sequence encoding UL78, or an ortholog thereof; and
(iii) the heterologous antigen replaces all or part of UL82 and is operably linked to the UL82 promoter.
4. A recombinant HCMV vector of embodiment 1, wherein:
(i) the vector does not express UL18, UL82, UL128, UL130, UL146, or UL 147, or orthologs thereof;
(ii) the vector comprises a nucleic acid sequence encoding UL78, or an ortholog thereof; and
(iii) the heterologous antigen replaces all or part of UL82 and is operably linked to the UL82 promoter.
5. The recombinant HCMV vector of any one of embodiments 1-4, wherein the vector does not express one or more of a UL18 protein, UL78 protein, UL82 protein, UL128 protein, UL130 protein, UL146 protein, or UL147 protein, resulting from the presence of one or more mutations in the nucleic acid sequence encoding UL18, UL78, UL82, UL128, UL130, UL146, or UL147.
6. The recombinant HCMV vector of embodiment 5, wherein the mutation in the nucleic acid sequence encoding UL18, UL78, UL82, UL128, UL130, UL146, or UL 147 is a point mutation, frameshift mutation, truncation mutation, or deletion of all of the nucleic acid sequence encoding the viral protein.
7. The recombinant HCMV vector of any one of embodiments 1-6, wherein the vector further comprises a nucleic acid sequence encoding a microRNA (miRNA) recognition element (MRE), wherein the MRE contains a target site for a miRNA expressed in endothelial cells.
8. The recombinant HCMV vector of any one of embodiments 1-7, wherein the vector further comprises a nucleic acid sequence encoding a MRE, wherein the MRE contains a target site for a miRNA expressed in myeloid cells.
9 The recombinant HCMV vector of any one of embodiments 1-8, wherein the heterologous antigen is a pathogen-specific antigen, a tumor antigen, a tissue-specific antigen, or a host self-antigen.
10. The recombinant HCMV vector of embodiment 9, wherein the pathogen is human immunodeficiency virus (HIV), herpes simplex virus type 1, herpes simplex virus type 2, hepatitis B virus, hepatitis C virus, papillomavirus, Plasmodium parasites, or Mycobacterium tuberculosis.
11. The recombinant HCMV vector of embodiment 9, wherein pathogen-specific antigen comprises a HIV antigen.
12. The recombinant HCMV vector of embodiment 11, wherein the HIV antigen is a fusion protein comprising or consisting of a HIV Gag, a HIV Nef, and a HIV Pol, or immunogenic fragments thereof, or combinations thereof.
13. The recombinant HCMV vector of embodiment 12, wherein the HIV antigen is a fusion protein comprising an amino acid sequence having at least 90%, at least 91%, at least 92%, at least 93%, at least 94%, at least 95%, at least 96%, at least 97%, at least 98%, at least 99%, or 100% identity to the amino acid sequence according to SEQ ID NO:3.
14. The recombinant HCMV vector of embodiment 12, wherein the HIV antigen is a fusion protein comprising the amino acid sequence according to SEQ ID NO: 3.
15. The recombinant HCMV vector of embodiment 12, wherein the HIV antigen is a fusion protein consisting of the amino acid sequence according to SEQ ID NO: 3.
16. The recombinant HCMV vector of embodiment 12, wherein the HIV antigen is a fusion protein comprising an amino acid sequence having at least 90%, at least 91%, at least 92%, at least 93%, at least 94%, at least 95%, at least 96%, at least 97%, at least 98%, at least 99%, or 100% identity to the amino acid sequence according to SEQ ID NO:4.
17. The recombinant HCMV vector of embodiment 12, wherein the HIV antigen is a fusion protein comprising the amino acid sequence according to SEQ ID NO: 4.
18. The recombinant HCMV vector of embodiment 12, wherein the HIV antigen is a fusion protein consisting of the amino acid sequence according to SEQ ID NO: 4.
19. The recombinant HCMV vector of embodiment 9, wherein the tumor antigen is related to acute myelogenous leukemia, chronic myelogenous leukemia, myelodysplastic syndrome, acute lymphoblastic leukemia, chronic lymphoblastic leukemia, non-Hodgkin's lymphoma, multiple myeloma, malignant melanoma, breast cancer, lung cancer, ovarian cancer, prostate cancer, pancreatic cancer, colon cancer, renal cell carcinoma (RCC), or germ cell tumors.
20. The recombinant HCMV vector of embodiment 9, wherein the host self-antigen is an antigen derived from the variable region of a T cell receptor (TCR) or an antigen derived from the variable region of a B cell receptor.
21. A recombinant HCMV vector comprising a nucleic acid sequence having at least 90%, 91%, 92%, 93%, 94%, 95%, 96%, 97%, 98%, 99%, or 100% identity to the nucleic acid sequence according to SEQ ID NO:7.
22. A recombinant HCMV vector comprising the nucleic acid sequence according to SEQ ID NO:7.
23. A recombinant HCMV vector consisting of the nucleic acid sequence according to SEQ ID NO:7.
24. A recombinant HCMV vector comprising a nucleic acid sequence having at least 90%, 91%, 92%, 93%, 94%, 95%, 96%, 97%, 98%, 99%, or 100% identity to the nucleic acid sequence according to SEQ ID NO:9.
25. A recombinant HCMV vector comprising the nucleic acid sequence according to SEQ ID NO:9.
26. A recombinant HCMV vector consisting of the nucleic acid sequence according to SEQ ID NO:9.
27. A recombinant HCMV vector comprising a nucleic acid sequence having at least 90%, at least 91%, at least 92%, at least 93%, at least 94%, at least 95%, at least 96%, at least 97%, at least 98%, at least 99%, or at least 100% identity to the nucleic acid sequence according to SEQ ID NO:5.
28. A recombinant HCMV vector comprising the nucleic acid sequence according to SEQ ID NO:5.
29. A recombinant HCMV vector consisting of the nucleic acid sequence according to SEQ ID NO:5.
30. A recombinant HCMV vector comprising a nucleic acid sequence having at least 90%, at least 91%, at least 92%, at least 93%, at least 94%, at least 95%, at least 96%, at least 97%, at least 98%, at least 99%, or at least 100% identity to the nucleic acid sequence according to SEQ ID NO:6.
31. A recombinant HCMV vector comprising the nucleic acid sequence according to SEQ ID NO:6.
32. A recombinant HCMV vector consisting of the nucleic acid sequence according to SEQ ID NO:6.
33. A recombinant HCMV vector comprising a nucleic acid sequence having at least 90%, at least 91%, at least 92%, at least 93%, at least 94%, at least 95%, at least 96%, at least 97%, at least 98%, at least 99%, or at least 100% identity to the nucleic acid sequence according to SEQ ID NO:8.
34. A recombinant HCMV vector comprising the nucleic acid sequence according to SEQ ID NO:8.
35. A recombinant HCMV vector consisting of the nucleic acid sequence according to SEQ ID NO:8.
36. A pharmaceutical composition comprising the recombinant HCMV vector of any one of embodiments 1-35 and a pharmaceutically acceptable carrier.
37. The pharmaceutical composition of embodiment 36, wherein the pharmaceutically acceptable carrier is a histidine trehalose (HT) buffer.
38. The pharmaceutical composition of embodiment 36 or 37, wherein the pharmaceutically acceptable carrier is a histidine trehalose (HT) buffer comprising about 20 mM L-histidine and about 10% (w/v) trehalose.
39. The pharmaceutical composition of any one of embodiments 36-38, wherein the pharmaceutically acceptable carrier is a histidine trehalose (HT) buffer comprising 20 mM L-histidine and 10% (w/v) trehalose.
40. The pharmaceutical composition of any one of embodiments 36-39, wherein the pharmaceutically acceptable carrier is a histidine trehalose (HT) buffer having a pH of 7.2 comprising 20 mM L-histidine and 10% (w/v) trehalose.
41. An immunogenic composition comprising the recombinant HCMV vector of any one of embodiments 1-35 and a pharmaceutically acceptable carrier.
42. A method of generating an immune response in a subject, comprising administering to the subject the recombinant HCMV vector or composition of any one of embodiments 1-41.
43. The method of embodiment 42, wherein the immune response is to the at least one heterologous antigen.
44. Use of the recombinant HCMV vector or composition of any one of embodiments 1-41 in the manufacture of a medicament for use in generating an immune response in a subject.
45. The recombinant HCMV vector or composition of any of embodiments 1-41 for use in generating an immune response in a subject.
46. A method of treating or preventing a disease in a subject, comprising administering the recombinant HCMV vector or composition of any one of embodiments 1-41.
47. A method of treating a disease in a subject, comprising administering the recombinant HCMV vector or composition of any one of embodiments 1-41.
48. A method of treating a disease in a subject, comprising administering the nucleic acid sequence according to SEQ ID NO:7 or pharmaceutical composition comprising the nucleic acid sequence according to SEQ ID NO:7.
49. A method of treating a disease in a subject, comprising administering the nucleic acid sequence according to SEQ ID NO:9 or pharmaceutical composition comprising the nucleic acid sequence according to SEQ ID NO:9.
50. A method of treating a disease in a subject, comprising administering the nucleic acid sequence according to SEQ ID NO:5 or pharmaceutical composition comprising the nucleic acid sequence according to SEQ ID NO:5.
51. A method of treating a disease in a subject, comprising administering the nucleic acid sequence according to SEQ ID NO:6 or pharmaceutical composition comprising the nucleic acid sequence according to SEQ ID NO:6.
52. A method of treating a disease in a subject, comprising administering the nucleic acid sequence according to SEQ ID NO:8 or pharmaceutical composition comprising the nucleic acid sequence according to SEQ ID NO:8.
53. A method of preventing a disease in a subject, comprising administering the recombinant HCMV vector or composition of any one of embodiments 1-41.
54. A method of preventing a disease in a subject, comprising administering the nucleic acid sequence according to SEQ ID NO:7 or pharmaceutical composition comprising the nucleic acid sequence according to SEQ ID NO:7.
55. A method of preventing a disease in a subject, comprising administering the nucleic acid sequence according to SEQ ID NO:9 or pharmaceutical composition comprising the nucleic acid sequence according to SEQ ID NO:9.
56. A method of preventing a disease in a subject, comprising administering the nucleic acid sequence according to SEQ ID NO:5 or pharmaceutical composition comprising the nucleic acid sequence according to SEQ ID NO:5.
57. A method of preventing a disease in a subject, comprising administering the nucleic acid sequence according to SEQ ID NO:6 or pharmaceutical composition comprising the nucleic acid sequence according to SEQ ID NO:6.
58. A method of preventing a disease in a subject, comprising administering the nucleic acid sequence according to SEQ ID NO:8 or pharmaceutical composition comprising the nucleic acid sequence according to SEQ ID NO:8.
59. Use of the recombinant HCMV vector or composition of any one of embodiments 1-41 in the manufacture of a medicament for use in treating or preventing a disease in a subject.
60. Use of the recombinant HCMV vector or composition of any one of embodiments 1-41 in the manufacture of a medicament for use in treating a disease in a subject.
61. Use of the nucleic acid sequence according to SEQ ID NO:7 or pharmaceutical composition comprising the nucleic acid sequence according to SEQ ID NO: 7 in the manufacture of a medicament for use in treating a disease in a subject.
62. Use of the nucleic acid sequence according to SEQ ID NO:9 or pharmaceutical composition comprising the nucleic acid sequence according to SEQ ID NO: 9 in the manufacture of a medicament for use in treating a disease in a subject.
63. Use of the nucleic acid sequence according to SEQ ID NO:5 or pharmaceutical composition comprising the nucleic acid sequence according to SEQ ID NO: 5 in the manufacture of a medicament for use in treating a disease in a subject.
64. Use of the nucleic acid sequence according to SEQ ID NO:6 or pharmaceutical composition comprising the nucleic acid sequence according to SEQ ID NO: 6 in the manufacture of a medicament for use in treating a disease in a subject.
65. Use of the nucleic acid sequence according to SEQ ID NO:8 or pharmaceutical composition comprising the nucleic acid sequence according to SEQ ID NO: 8 in the manufacture of a medicament for use in treating a disease in a subject.
66. Use of the recombinant HCMV vector or composition of any one of embodiments 1-41 in the manufacture of a medicament for use in preventing a disease in a subject.
67. Use of the nucleic acid sequence according to SEQ ID NO:7 or pharmaceutical composition comprising the nucleic acid sequence according to SEQ ID NO: 7 in preventing a disease in a subject.
68. Use of the nucleic acid sequence according to SEQ ID NO:9 or pharmaceutical composition comprising the nucleic acid sequence according to SEQ ID NO: 9 in preventing a disease in a subject.
69. Use of the nucleic acid sequence according to SEQ ID NO:5 or pharmaceutical composition comprising the nucleic acid sequence according to SEQ ID NO: 5 in preventing a disease in a subject.
70. Use of the nucleic acid sequence according to SEQ ID NO:6 or pharmaceutical composition comprising the nucleic acid sequence according to SEQ ID NO: 6 in preventing a disease in a subject.
71. Use of the nucleic acid sequence according to SEQ ID NO:8 or pharmaceutical composition comprising the nucleic acid sequence according to SEQ ID NO: 8 in preventing a disease in a subject.
72. The recombinant HCMV vector or composition of any one of embodiments 1-41 for use in treating or preventing a disease in a subject.
73. The recombinant HCMV vector or composition of any one of embodiments 1-41 for use in treating a disease in a subject.
74. The nucleic acid sequence according to SEQ ID NO:7 or pharmaceutical composition comprising the nucleic acid sequence according to SEQ ID NO: 7 for use in treating a disease in a subject.
75. The nucleic acid sequence according to SEQ ID NO:9 or pharmaceutical composition comprising the nucleic acid sequence according to SEQ ID NO: 9 for use in treating a disease in a subject.
76. The nucleic acid sequence according to SEQ ID NO:5 or pharmaceutical composition comprising nucleic acid sequence according to SEQ ID NO: 5 for use in treating a disease in a subject.
77. The nucleic acid sequence according to SEQ ID NO:6 or pharmaceutical composition comprising the nucleic acid sequence according to SEQ ID NO: 6 for use in treating a disease in a subject.
78. The nucleic acid sequence according to SEQ ID NO:8 or pharmaceutical composition comprising the nucleic acid sequence according to SEQ ID NO: 8 for use in treating a disease in a subject.
79. The recombinant HCMV vector or composition of any one of embodiments 1-41 for use in preventing a disease in a subject.
80. The nucleic acid sequence according to SEQ ID NO:7 or pharmaceutical composition comprising the nucleic acid sequence according to SEQ ID NO: 7 for use in preventing a disease in a subject.
81. The nucleic acid sequence according to SEQ ID NO:9 or pharmaceutical composition comprising the nucleic acid sequence according to SEQ ID NO: 9 for use in preventing a disease in a subject.
82. The nucleic acid sequence according to SEQ ID NO:5 or pharmaceutical composition comprising the nucleic acid sequence according to SEQ ID NO: 5 for use in preventing a disease in a subject.
83. The nucleic acid sequence according to SEQ ID NO:6 or pharmaceutical composition comprising the nucleic acid sequence according to SEQ ID NO: 6 for use in preventing a disease in a subject.
84. The nucleic acid sequence according to SEQ ID NO:8 or pharmaceutical composition comprising the nucleic acid sequence according to SEQ ID NO: 8 for use in preventing a disease in a subject.
85. The method, use in manufacture, or vector or composition for use of any one of embodiments 42-84, wherein the subject is seropositive for HCMV.
86. The method, use in manufacture, or vector or composition for use of any one of embodiments 42-84, wherein the subject is seronegative for HCMV.
87. The method, use in manufacture, or vector or composition for use of any one of embodiments 42-86, wherein the recombinant HCMV is administered in an amount of at least 1×10³ focus-forming units (ffu).
88. The method, use in manufacture, or vector or composition for use of embodiment 87, wherein the recombinant HCMV is administered in an amount of about 5×10⁵ ffu.
89. The method, use in manufacture, or vector or composition for use of embodiment 87, wherein the recombinant HCMV is administered in an amount of about 5×10⁵ ffu.
90. The method, use in manufacture, or vector or composition for use of embodiment 87, wherein the recombinant HCMV is administered in an amount of about 5×10⁶ ffu.
91. The method, use in manufacture, or vector or composition for use of embodiment 87, wherein the recombinant HCMV is administered in an amount of about 1×10³ ffu.
92. The method, use in manufacture, or vector or composition for use of embodiment 87, wherein the recombinant HCMV is administered in an amount of about 3×10⁴ ffu.
93. The method, use in manufacture, or vector or composition for use of embodiment 87, wherein the recombinant HCMV is administered in an amount of about 1×10⁶ ffu.
94. The method use in manufacture, or vector or composition for use of any one of embodiments 42-93, wherein the recombinant HCMV vector is administered in an amount effective to elicit a CD8+ T cell response to the at least one heterologous antigen.
95. The method, use in manufacture, or vector or composition for use of any one of embodiments 42-94, wherein the heterologous antigen is or comprises a HIV antigen and the disease is HIV infection.
96. The method, use in manufacture, or vector or composition for use of any one of embodiments 42-94, wherein the disease is a pathogenic infection, a tumor or cancer, or an autoimmune disease.
97. The method, use in manufacture, or vector or composition for use of any one of embodiments 63-96, wherein at least 10% of the CD8+ T cells elicited by the recombinant HCMV vector are restricted by MHC-E or an ortholog thereof.
98. The method, use in manufacture, or vector or composition for use of any one of embodiments 63-97, wherein at least 20%, at least 30%, at least 40%, at least 50%, at least 60%, at least 75%, at least 80%, at least 85%, at least 90%, or at least 95% of the CD8+ T cells elicited by the recombinant HCMV vector are restricted by MHC-E or an ortholog thereof.
99. The use in manufacture, or vector or composition for use of any one of embodiments 63-98, wherein at least 10% of the CD8+ T cells elicited by the recombinant HCMV vector are restricted by MHC-II or an ortholog thereof.
100. The method, use in manufacture, or vector or composition for use of any one of embodiments 63-99, wherein at least 20%, at least 30%, at least 40%, at least 50%, at least 60% or at least 75% of the CD8+ T cells elicited by the recombinant HCMV vector are restricted by MHC-II or an ortholog thereof.
101. The method, use in manufacture, or vector or composition for use of any one of embodiments 63-100, wherein fewer than 10%, fewer than 20%, fewer than 30%, fewer than 40%, or fewer than 50% of the CD8+ T cells elicited by the recombinant HCMV vector are restricted by MHC-class Ia or an ortholog thereof.
102. A method of generating CD8+ T cells that recognize MHC-E/peptide complexes, the method comprising:
(a) administering to a first subject the recombinant HCMV vector of any one of embodiments 1-35 in an amount effective to generate a set of CD8+ T cells that recognize MHC-E/heterologous antigen-derived peptide complexes;
(b) identifying a first CD8+ TCR from the set of CD8+ T cells, wherein the first CD8+ TCR recognizes a MHC-E/peptide complex;
(c) isolating one or more CD8+ T cells from a second subject; and
(d) transfecting the one or more CD8+ T cells isolated from the second subject with an expression vector, wherein the expression vector comprises a nucleic acid sequence encoding a second CD8+ TCR and a promoter operably linked to the nucleic acid sequence encoding the second CD8+ TCR, wherein the second CD8+ TCR comprises CDR3a and CDR3B of the first CD8+ TCR, thereby generating one or more CD8+ T cells that recognize MHC-E/peptide complexes.
103. A method of generating CD8+ T cells that recognize MHC-E/peptide complexes, the method comprising:
(a) identifying a first CD8+ TCR from a set of CD8+ T cells, wherein the set of CD8+ T cells are isolated from a first subject that has been administered the recombinant HCMV vector of any one of embodiments 1-35, and wherein the first CD8+ TCR recognizes a MHC-E/heterologous antigen-derived peptide complex;
(b) isolating one or more CD8+ T cells from a second subject; and
(c) transfecting the one or more CD8+ T cells isolated from the second subject with an expression vector, wherein the expression vector comprises a nucleic acid sequence encoding a second CD8+ TCR and a promoter operably linked to the nucleic acid sequence encoding the second CD8+ TCR, wherein the second CD8+ TCR comprises CDR3a and CDR3B of the first CD8+ TCR, thereby generating one or more TCR-transgenic CD8+ T cells that recognize MHC-E/peptide complexes.
104. The method of embodiment 102 or 103, wherein the first CD8+ TCR is identified by DNA or RNA sequencing.
105. The method of any one of embodiments 102-104, wherein the nucleic acid sequence encoding the second CD8+ TCR is identical to the nucleic acid sequence encoding the first CD8+ TCR.
106. The method of any one of embodiments 102-105, wherein the first subject is a human.
107. The method of any one of embodiments 102-106, wherein the first subject is seropositive for HCMV.
108. The method of any one of embodiments 102-106, wherein the first subject is seronegative for HCMV.
109. The method of any one of embodiments 102-108, wherein the second subject is a human.
110. A CD8+ T cell generated by the method of any one of embodiments 102-109.
111. A method of treating or preventing a disease in a subject, the method comprising administering the CD8+ T cell of embodiment 110 to the subject.
112. A method of treating a disease in a subject, the method comprising administering the CD8+ T cell of embodiment 110 to the subject.
113. A method of preventing a disease in a subject, the method comprising administering the CD8+ T cell of embodiment 110 to the subject.
114. Use of the CD8+ T cell of embodiment 110 in the manufacture of a medicament for use in treating or preventing a disease in a subject.
115. Use of the CD8+ T cell of embodiment 110 in the manufacture of a medicament for use in treating a disease in a subject.
116. Use of the CD8+ T cell of embodiment 110 in the manufacture of a medicament for use in preventing a disease in a subject.
117. The CD8+ T cell of embodiment 110 for use in treating or preventing a disease in a subject.
118. The CD8+ T cell of embodiment 110 for use in treating a disease in a subject.
119. The CD8+ T cell of embodiment 110 for use in preventing a disease in a subject.

### Examples

### Example 1: Immunogenicity Experiments in Non-Human Primates

Vaccine vectors based on Cytomegalovirus (CMV) exploit the natural ability of this virus to elicit and maintain circulating and tissue resident effector-differentiated T cells, including the potential sites of early HIV infection. For example, rhesus CMV (RhCMV) vectors encoding simian immunodeficiency virus (SIV) antigen inserts can (1) superinfect RhCMV-immune primates and elicit high frequency effector-differentiated, SIV-specific CD4+ and CD8+ T cells in both lymphoid and organ tissues, (2) maintain these responses indefinitely, and (3) manifest early stringent control and ultimate clearance of infection with the highly pathogenic SIVmac239 strain. A prophylactic HIV vaccine is developed that stimulates the induction and maintenance of high frequencies of HIV-specific CD8+ T cells with the goal of broad epitope coverage to avoid selection of cytotoxic T cell escape variants and T cell exhaustion characteristic of HIV-specific T cells in patients. The vaccine is tested in rhesus macaques and/or cynomolgus monkeys.

### Example 2: Clinical Evaluation of an HCMV-Based HIV Vaccine

An HIV vaccine will be tested in a first-in-human, Phase 1a, randomized, multiple-site, double-blind, placebo-controlled study in healthy adult volunteers from ages 18 to 50 who are CMV seropositive and HIV uninfected. The vaccine is a live, attenuated human CMV vector (Vector 1) that expresses the HIV-1 clade A gag gene.

Although the number of new HIV infections per year has decreased, it remains high with 1.8 million new infections occurring in 2017 alone, and annual mortality due to HIV/acquired immunodeficiency syndrome (AIDS) continues to remain high at approximately 0.9 million deaths worldwide per year (UNAIDS/WHO Data 2018, https://www.unaids.org/sites/default/files/media_asset/unaids-data-2018_en.pdf). The failure of all prior HIV candidate vaccines to achieve efficacy suggests that protection may require vaccine-elicited immunity that differs qualitatively from previous vaccine strategies. An ideal HIV vaccine would not only deliver relevant HIV antigens to the immune system, but these antigens would also be expressed in a vector that has the capacity to govern how the immune system responds to these antigens, a concept that has been termed “antigen delivery and immune programming (ADIP)”.

CMV has long been known to elicit a robust immune response, characterized by the life-long maintenance of high frequency virus-specific T cells, predominantly of the effector memory (T_{EM}) phenotype, that are capable of trafficking to tissues and manifesting immediate anti-viral effector responses. As antigen-specific T_{EM} cells have a shorter half-life than T_{CM} cells, a vector that persistently presents antigens in the host and can provide long-term replenishment of these cells is ideal. A major step forward in the goal of generating a protective HIV vaccine was made in 2011 with the report that an RhCMV-based vaccine encoding simian immunodeficiency virus (SIV) antigens was able to protect approximately 50% of rhesus macaques (RM) from establishing sustained infection following repeated low dose mucosal exposure to the highly pathogenic SIV_{mac239} (Hansen S G et al., Profound early control of highly pathogenic SIV by an effector memory T cell vaccine, Nature 2011; 473(7348): 523-7). It was also demonstrated that RhCMV had the unique quality of being able to elicit and maintain populations of highly functional CD4+ and CD8+ memory T cells. In animals that showed protection against SIV challenge, stringent immunologic control was achieved very early after the onset of infection and, in the vast majority, resulted in complete viral clearance as determined by both long-term follow-up and sensitive laboratory methods for the detection of SIV in tissues. A series of reports by Picker et al. confirmed the potential of a CMV-based vaccine to elicit broad and durable cellular responses capable of stringently controlling SIV infection following exposure. Subsequent work has demonstrated the reproducibility of this result and revealed the central importance of unconventionally restricted (MHC-E and MHC-II) CD8+ T cell effector responses as a key immunologic mechanism associated with protection, as opposed to conventional MHC-Ia restricted CD8+ T cells (Hansen S G et al., Cytomegalovirus vectors violate CD8+ T cell epitope recognition paradigms, Science 2013; 340(6135): 1237874; Marshall E et al, Enhancing Safety of cytomegalovirus-based vaccine vectors by engaging host intrinsic immunity, Science Translational Medicine 2019 Jul. 17; 11(501)). This concept of immune programming—that genetic manipulation of CMV vectors can preferentially induce unconventionally restricted CD8+ T cells-represents a new paradigm in vaccine development.

In this study, the vaccine is used to determine whether the immune programming associated with similar RhCMV vectors in RMs can be recapitulated in humans. It contains an antigenic cassette encoding HIV gag that is specifically designed to provide persistent antigen presentation in order to evaluate whether the immune response elicited by this HCMV vector is skewed toward a cellular immune profile similar to that associated with protection against SIV in RMs.

The study will be conducted as a 3-cohort dose escalation. A Safety Review Committee (SRC) will perform periodic reviews of safety, reactogenicity, and tolerability based on available study data collected throughout the study with the primary purpose of protecting the safety of subjects participating in the clinical study. The SRC will perform safety data review prior to dose initiation in the next cohort.

### Objectives

The primary objective of the study is to evaluate the safety, reactogenicity, and tolerability of the vaccine compared to placebo when administered subcutaneously in healthy CMV seropositive adult subjects. The secondary objective is to characterize the immunogenicity of the vaccine as measured by T cell and antibody responses to vaccine derived HIV-1 Gag. Exploratory objectives may include: (1) to further characterize immune responses to the vaccine by analyses of the MHC molecule type for CD8+ T cell recognition of vaccine derived HIV Gag, T cell receptor repertoire mediating this recognition, ability of vaccine elicited CD8+ T cells to responds to HIV-infected cells, and other T cell functional and phenotypic measures; (2) to identify a transcriptomic “signature” profile in peripheral whole blood imparted by administration of the vaccine; (3) to characterize the immunogenicity of the vaccine as measured by T cell and antibody responses to CMV.

### Endpoints

The primary endpoint(s) of this study are incidence of treatment emergent AEs, SAEs and NOCDs; incidence of local site or systemic reactogenicity events; and clinical assessments including but not limited to laboratory test results, CMV vector viremia, and CMV vector shedding. The secondary endpoints of this study are to assess magnitude, function, and phenotypic profile of insert-specific CD4+ and CD8+ T cell responses as assessed by intracellular cytokine staining and flow cytometry and measure serologic titer of HIV-1 Gag-specific antibodies. The exploratory endpoints of this study may include assessment of the breadth of HIV Gag specific T cell epitopes generated in response to the vaccine, CD8+ T cell restriction assignment generated in response to the vaccine, functional capacity of T cell mediated recognition of HIV-infected target cells in response to the vaccine, characterization of HIV Gag-specific T cell repertoire generated by the vaccine through TCR clonotyping, changes in magnitude and phenotypic profile of CMV-specific CD4+ and CD8+ T cell responses, changes in the serologic titer of CMV-specific antibodies, and HIV vaccine induced seropositivity (VISP) in response to the vaccine.

### Active Agent and Dosing

The vaccine is a live, attenuated human CMV vector expressing the HIV-1 clade A gag gene. The recombinant HCMV vector is derived from clinical isolate TR (Smith I L et al., High-level resistance of cytomegalovirus to ganciclovir is associated with alterations in both the UL97 and DNA polymerase genes. J Infect Dis. 1997; 176(1): 69-77). To create the vector, TR was genetically modified to restore ganciclovir susceptibility and MHC-I inhibitory activity. The UL82 gene encoding the tegument protein pp71 was deleted in the vector and replaced with an antigenic cassette encoding the HIV-I Clade A gag transgene (Keefer M C et al. A phase I double blind, placebo-controlled, randomized study of a multigenic HIV-1 adenovirus subtype 35 vector vaccine in healthy uninfected adults, PLOS ONE 2012; 7(8): e41936). The vector incorporates multiple attenuation strategies including the deletion of the UL82 gene and deletion of the pentameric complex components UL128-130, which control host cell tropism. Deletion of UL128-130 and UL146-147 genes may influence the characteristics of the host CD8+ T cell response.

Each single use vial contains 0.5 mL vector in TNS (50 mM Tris, 150 mM NaCl, 10% sucrose) formulation buffer. Each dose will be administered as a 1 mL SC injection in the deltoid area of the upper arm. The starting dose is 1×10³ focus forming units (ffu). Subjects randomized to placebo will receive 1 mL TNS formulation buffer (vehicle) via SC injection. Each single use vial contains 0.5 mL TNS formulation buffer.

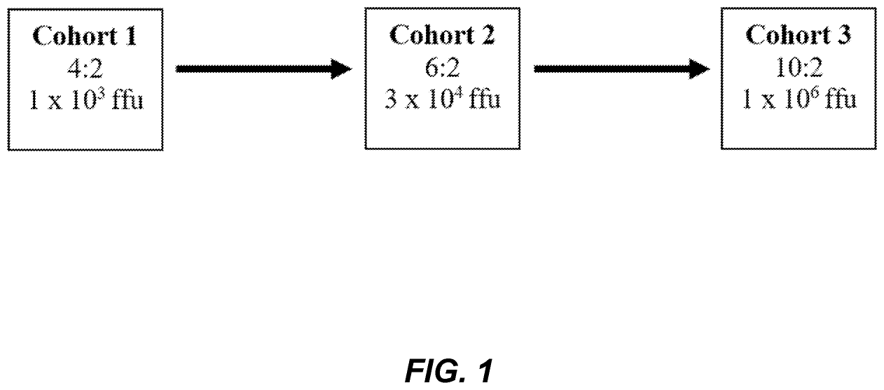

A total of up to 26 subjects will be enrolled into 3 ascending dose cohorts of the vaccine administered subcutaneously (see Table 1 below and FIG. 1). Cohort 1 will consist of 6 subjects randomized 4:2 to vaccine or placebo. Cohort 2 will consist of 8 subjects randomized 6:2 to vaccine or placebo. Cohort 3 will consist of 12 subjects randomized 10:2 to vaccine or placebo. The initial starting dose will be 1×10³ focus forming units (ffu). The dose in subsequent cohorts will be increased stepwise in ˜30-fold increments up to 1×10⁶ ffu, a dose range well tolerated with no safety signals in preclinical GLP toxicology studies. All subjects will undergo follow-up monitoring and testing as outlined in the Schedule of Assessments (SOA). Subjects will receive a second subcutaneous dose on Day 57. The second dose will be the same product dosage level received during the first dose.

Progression to the next cohort will occur only after available safety data through the last subject's Week 8 visit (including adverse events, vital signs, and clinical laboratory results) and all previously dosed subjects in the preceding lower dose level(s), have been evaluated and approved by the SRC. An 8-week interval was selected based on prior attenuated CMV vaccine studies that demonstrated that no further immunologic or systemic effects of the vaccine occurred after 8 weeks.

TABLE 1
Dosing regimens for each cohort.

| | | Dosage Administered | |
|---|---|---|---|
| | Cohort | on Day 1 and Day 57 | Randomization |
| Dose | 1 | 1 × 10³ ffu | 4 Vaccine |
| Escalation | | | 2 Placebo |
| | 2 | 3 × 10⁴ ffu | 6 Vaccine |
| | | | 2 Placebo |
| | 3 | 1 × 10⁶ ffu | 10 Vaccine |
| | | | 2 Placebo |

| | |
|---|---|
| Total Subjects | 26 (20 vaccine/ |

6 Placebo)

### Screening

Screening will be performed no more than 56 days prior to Day 1 visit and will include written consent, determination of eligibility, collection of demographics and medical history, physical examination (including vital signs), laboratory tests, and other assessments. Adverse events (AEs) related to screening activities must be collected from the time of consent onwards; any other events occurring during the screening period should be reported as medical history. All serious adverse events (SAEs) must be collected from the time of consent onwards.

### Inclusion and Exclusion Criteria

Each subject must meet all of the following inclusion criteria to be eligible for enrollment in the study: (1) Healthy males, or healthy females of non-childbearing potential, between the ages of 18 to 50 at the time of screening. Transgender individuals may be enrolled if they meet non-childbearing potential and laboratory value requirements based on sex assigned at birth with exceptions for individuals on hormone therapy; (2) positive CMV serostatus; (3) assessed by clinic staff as being low risk for HIV infection and committed to maintaining behavior consistent with low risk of HIV exposure through the last protocol visit; (4) willing to use condoms during intercourse through week 36 or the end of the study; (5) willing to undergo HIV testing, risk reduction counseling, and receive HIV test results; (6) willing to refrain from donating blood, sperm, or other tissues during the study; (7) in the opinion of the site investigator, the subject is generally in good health as determined from medical history and no significant findings from a physical examination, vital signs, and laboratory values; (8) willing to comply with the requirements of the protocol and be available for follow up for the planned duration of the study; and (9) able to provide written informed consent.

Low risk for HIV infection is considered to be: no personal history of injection drug use within 3 years of screening and none of the following within 1 year prior to the study: personal history of sexually transmitted disease, sex with an HIV-infected individual, sex with an active injection drug user, inconsistent condom use, unprotected sexual activity with unknown partner(s), and participation in commercial sex work.

For the purpose of this document, a woman is considered of childbearing potential (WOCBP) following menarche and until becoming post-menopausal unless permanently sterile. Permanent sterilization methods include hysterectomy, bilateral salpingectomy, and bilateral oophorectomy. A postmenopausal state is defined as no menses for 12 months without an alternative medical cause. For the purpose of this document, a man is considered fertile after puberty unless permanently sterile by bilateral orchiectomy with documented azoospermia.

Each subject must not meet any of the following exclusion criteria to be eligible for enrollment in the study: (1) Live in a home with children under the age of 6; (2) routine provision of child care to children under the age of 6; (3) have close contact with immunocompromised individuals; (4) have close contact with pregnant women or a partner planning to become pregnant during the course of the study; (5) health care provider who routinely comes into contact with immunosuppressed patients or pregnant women; (6) subject is immunocompromised; (7) subject has an autoimmune disorder; (8) positive human immunodeficiency virus (HIV) test at the time of screening; (9) cancer or history of cancer within the last 5 years, except for non-invasive cancers resolved with local therapy such as excised basal cell cancer; (10) current active or chronic hepatitis B or hepatitis C infection by laboratory test at screening; (11) seizure disorder with any seizure in the last 3 years; (12) any clinically significant chronic medical condition that, in the opinion of the principal investigator makes the volunteer unsuitable for participation in the study; (13) subject has alanine transaminase (ALT) 1.2×above the upper limit of normal (ULN), aspartate transaminase 1.2×above the ULN, direct or total bilirubin 1.1×above the ULN, alkaline phosphatase 1.1×above the ULN, gamma-glutamyl transferase 1.1×above the LN, creatinine 1.1×above the ULN, hemoglobin levels less than or equal to 11.0 g/dL who were assigned female sex at birth, hemoglobin levels less than or equal to 13.0 g/dL who were assigned male sex at birth, platelets more than 20,000 above the ULN or lower limit of normal (LLN), white blood cells more than 1,000 cells/mL³ above the ULN or below the LLN; (14) poor venous access as assessed by the investigator, (15) previous severe local or systemic reactogenicity to vaccines; (16) any clinically significant acute infection or acute respiratory illness within 14 days of first dose; (17) use of (val) acyclovir, (val) ganciclovir, letermovir, foscarnet, or another antiviral with anti-CMV activity within 30 days prior to the first does of IP and/or any use anticipated, based on medical history, through week 16 of the study (or at least 8 weeks after receipt of second dose); (18) receipt of any live-attenuated vaccines within 30 days prior to the first dose of IP and/or anticipated through week 16 of the study (or at least 8 weeks after receipt of the second dose); (19) receipt of any mRNA-containing coronavirus vaccine within 30 days prior to first dose of IP; (20) receipt of the inactivated influenza vaccine or other inactivated/subunit vaccines within the first 14 days of the first dose of IP; (21) tuberculin skin test or allergy treatment with antigen injections within the previous 14 days or planned receipt within 14 days after the first dose of IP; (22) receipt of blood transfusion or blood-derived products within the previous 6 months or expectation of receiving blood products during the study period; (23) any participation in another clinical trial of an IP within the previous 3 months or expected participation during the study (receipt of placebo is not exclusionary); (24) receipt of another investigational HIV or CMV vaccine candidate; (25) planned use of any prohibited concomitant medication as defined above; (26) previous or current psychiatric condition that precludes compliance with the protocol; (27) significant alcohol or drug use in the opinion of the investigator that precludes compliance with the protocol and/or compromises subject safety; (28) a positive drug screen (i.e., for cocaine, barbiturates, benzodiazepines, or amphetamines) will exclude subjects unless the positive test can be explained by a prescribed medication.

The following medications and/or treatments are prohibited through Week 36 of the study: (1) Immunosuppressive medications including but not limited to corticosteroids, calcineurin inhibitors, mTor inhibitors, IMDH inhibitors, or immunosuppressive biologics; (2) use of valacyclovir, valganciclovir, letermovir, foscarnet, or another antiviral with anti-CMV activity within 30 days before the first IP dose and through Week 16 of the study, or at least 8 weeks after receipt of the second IP dose; (3) receipt of the inactivated influenza vaccine or other inactivated/subunit vaccines within 14 days of the first or second dose of IP; (4) receipt of any mRNA-containing coronavirus vaccine within 30 days prior to the first dose of IP; (5) receipt of any live-attenuated vaccines within 30 days prior to the first dose of IP and through week 16 of the study, or at least 8 weeks after receipt of the second dose; (6) tuberculin skin test or allergy treatment with antigen injections within the previous 14 days or planned receipt within 14 days after the first or second dose of IP; (7) receipt of another investigational HIV or CMV vaccine candidate.

Corticosteroid nasal sprays for allergic rhinitis, topical corticosteroids for mild dermatitis unrelated to the injection site, oral/parenteral corticosteroids given for non-chronic conditions not expected to recur with length of therapy 10 days or less and completed at least 30 days prior to enrollment are permitted. If a single treatment of corticosteroids for an acute condition is considered during the study, consultation with the Vir Medical Monitor is required.

Women of child-bearing potential may not be enrolled in the study. Post-menopausal women are allowed to participate. Male subjects with female partners of child-bearing potential must agree to meet one of the following contraception requirements from the time of study treatment administration until the last follow-up visit: (1) Male condoms and a vasectomy with documentation of azoospermia or (2) male condom plus partner use of one additional birth control method. If the partner of a male subject becomes pregnant from the time of IP administration through 36 weeks after the last dose, the subject will be instructed to report this to the investigator. The investigator must report the pregnancy to the sponsor or designee within 24 hours of being notified of the pregnancy. The partner of the male subject will be asked to provide consent to be followed until the outcome of the pregnancy and for up to 1 year after birth, where permitted.

All subjects are prohibited from donating blood, sperm, or other tissues during the study. At the end of the study, clinical guidance regarding donation will be provided based on the results of the study.

### Treatment Period (Day 1 to Day 57)

Eligibility, criteria, medical history, and screening lab results will be reviewed on Day 1. Eligible subjects will be randomized to vaccine or matching placebo within 48 hours prior to Day 1 investigation product (IP) administration. Subjects will return to the clinical investigative site on Day 57 (Week 8) to receive a second dose of the same IP and dose administered on Day 1. Following IP injection, subjects will remain in the clinic for at least 30 minutes of observation. A reactogenicity assessment will be performed at 30 minutes (acceptable range 25-60 minutes). The reactogenicity assessment will include vital signs and injection site inspection and documentation for evidence of local reaction. Subjects will be given a diary card to use as a memory aid for daily documentation of symptoms of local and systemic reactogenicity for 14 days following receipt of each dose.

### Post-Dose Follow-Up Period

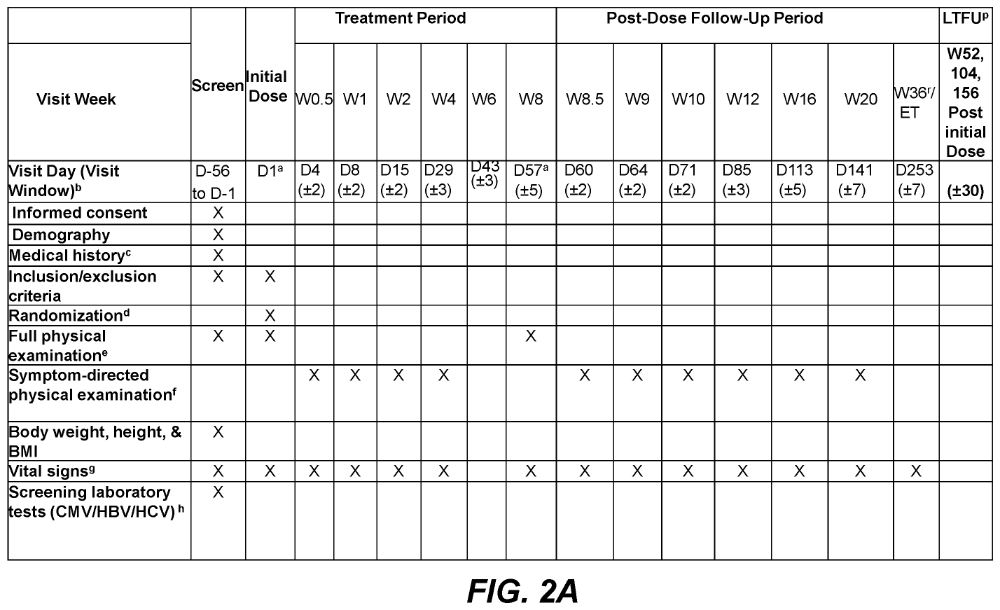

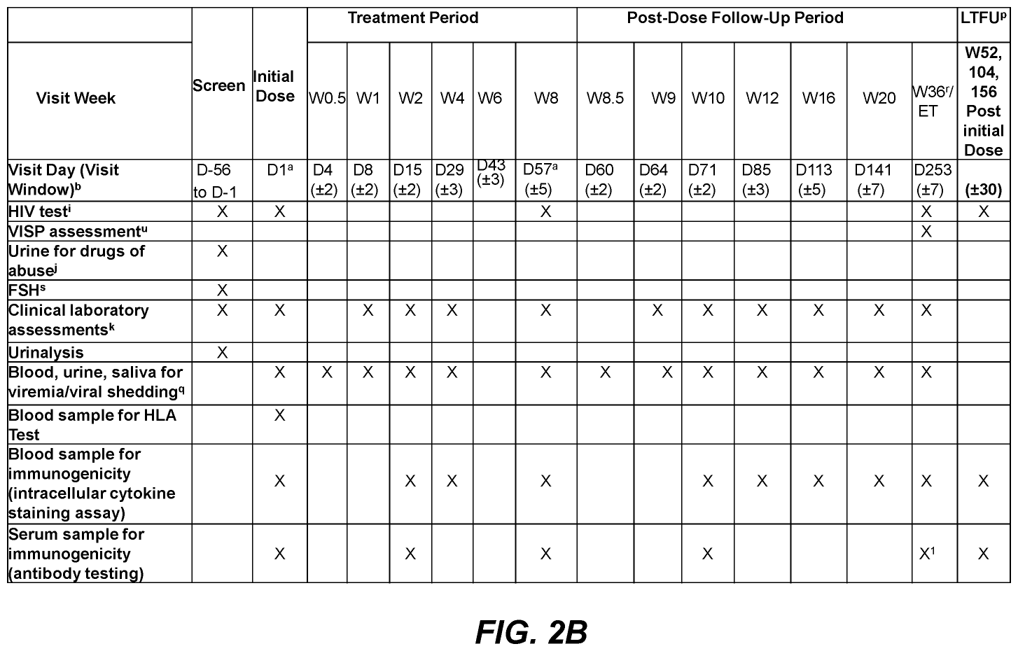

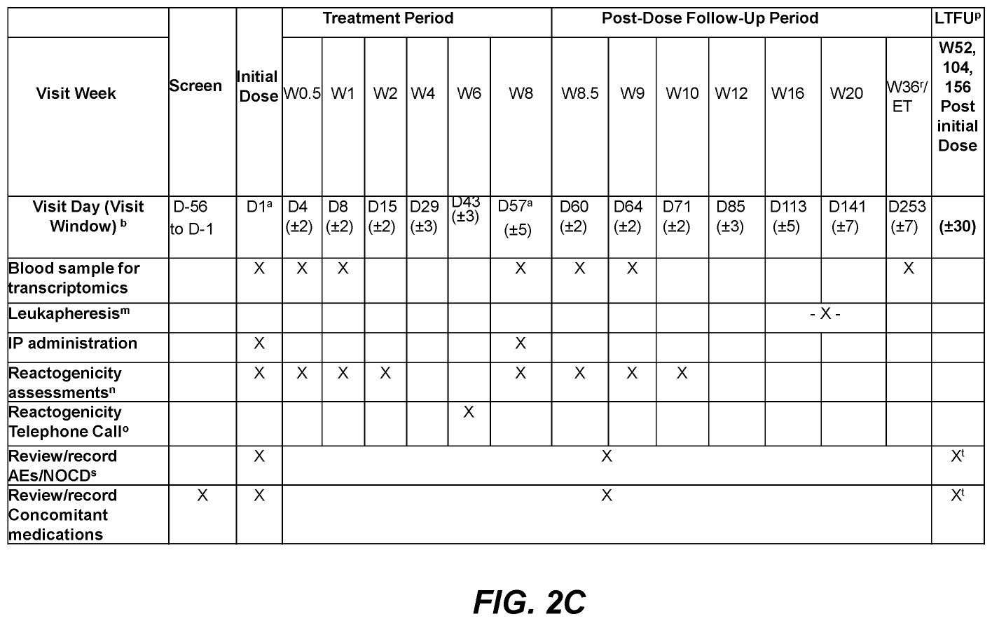

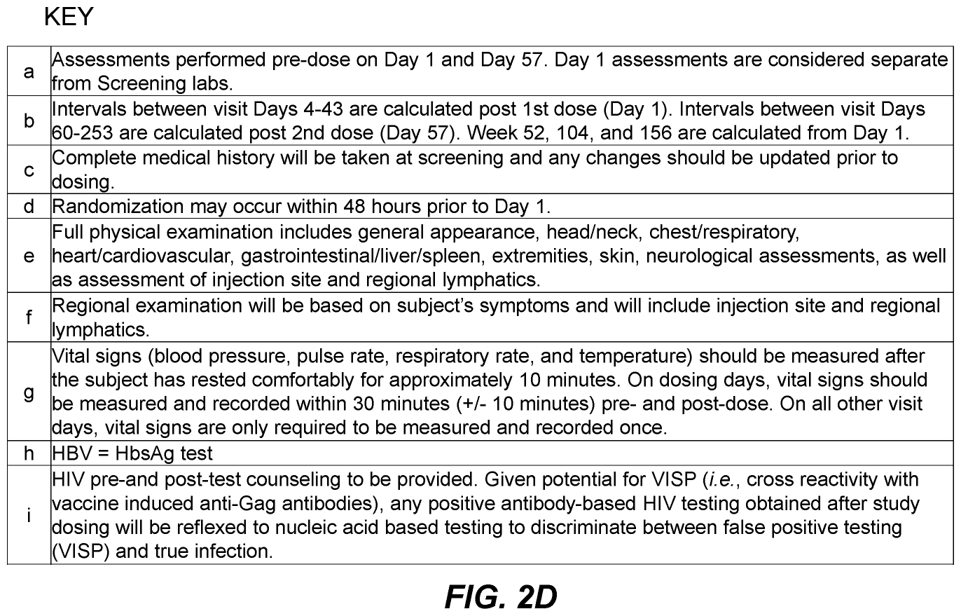

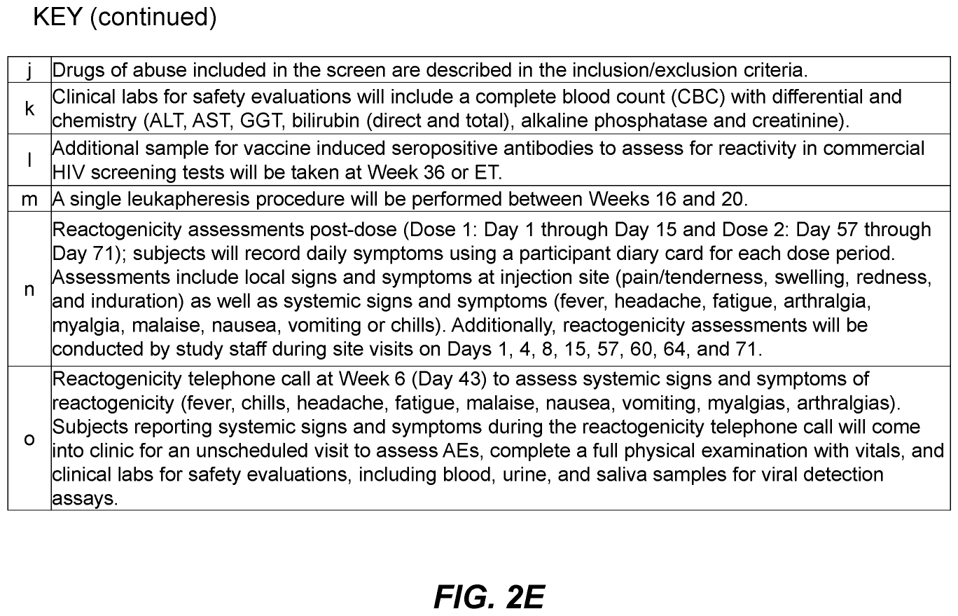

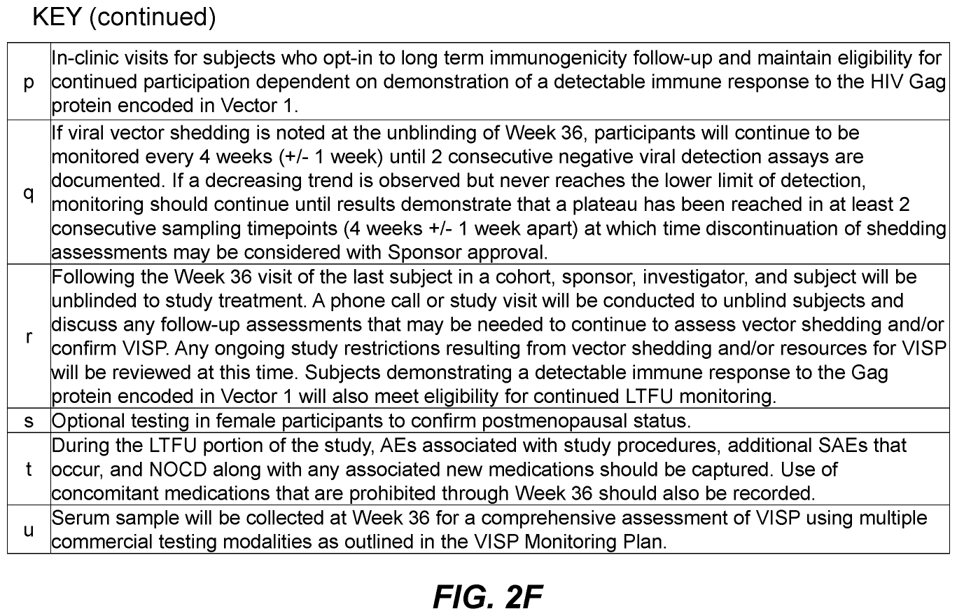

Subjects will return to the clinic for in-person assessments according to the SoA (see FIGS. 2A-2F) including but not limited to physical examination with vital signs, laboratory testing for safety and immunogenicity, and review of AEs and concomitant medications(s).

AEs and clinical laboratory abnormalities will be assessed using the Division of AIDS (DAIDS) Table for Grading the Severity of Adult and Pediatric Adverse Events.

Each cohort will be unblinded after the last subject in each cohort complete the Week 36 visit in order to assess the presence of ongoing viral vector shedding and vaccine induced seropositivity (VISP). Participants who demonstrate ongoing viral vector shedding at the end of the study will be evaluated and followed as outlined in the SoA. Participants who develop VISP will be evaluated and followed.

### Optional Long-Term Follow-Up (LTFU)

Participants will have the option to participate in a 3-year LTFU. For participants providing additional consent, an annual clinic visit will be conducted for sample collection to monitor long-term immunogenicity and general health status. Eligibility for continued participation in the LTFU portion of the study may be dependent on a participant demonstrating a detectable immune response to the HIV Gag protein encoded in the vaccine. Given the turnaround time for results of immunogenicity assessments, consenting participants may begin LTFU assessments before confirmatory immunogenicity results are available. Eligibility to continue participating in LTFU will be confirmed following the availability and subsequent unblinding of immunogenicity data.

### Discontinuations

Subjects who discontinue IP prematurely will be followed for safety and immunogenicity as outlined in the SoA, and under certain circumstances, subjects who discontinue IP at any point prior to completing leukapheresis may be replaced. If a subject discontinues from the study post-second dose but before completion of the study at Week 36, an Early Termination (ET) visit be performed.

If a subject discontinues from the study, for example as a result of an AE, every attempt will be made to keep the subject in the study and continue to perform the required study-related procedures until stabilization, per the investigator. If this is not possible or acceptable to the subject or investigator, the subject may be withdrawn from the study. Evaluations indicating abnormal results believed to be possibly or probably related to study treatment at the ET visit should be repeated weekly or as often as deemed appropriate by the investigator until the abnormality resolves, returns to baseline visit levels, or is otherwise explained.

If the subject withdraws from the study, the ET evaluations and/or procedures outlined in the SoA should be performed within 7 days of the subject permanently discontinuing from the study.

### Stopping Criteria

Enrollment will be halted if one or more of the following criteria are met: (1) If two or more subjects experience the same treatment related Grade 3 or higher adverse event; if one subject experiences a treatment-related SAE; if one subject experiences documented end-organ disease attributable to the CMV vector other than mild, self-limited mononucleosis-like syndrome, as determined by signs, symptoms, laboratory findings and detection of vaccine vector in relevant site(s).

When a stopping criteria is met, no further IP will be administered at the dose level for the affected cohort and further dose escalation/progression will be suspended. An ad hoc Safety Review Committee meeting will be held to review available safety data from all cohorts and to provide a recommendation regarding continuation of dosing.

Individual subjects who have received one dose of the vaccine will not receive a second dose if any of the following criteria are met: (1) Interval development of a clinically significant condition; (2) experienced any Grade 3 or higher treatment-related adverse event and/or a type 1 hypersensitivity reaction associated with the vaccine; (3) inability to receive a dose within the specified period for the study visit.

### Assessments and Study Procedures

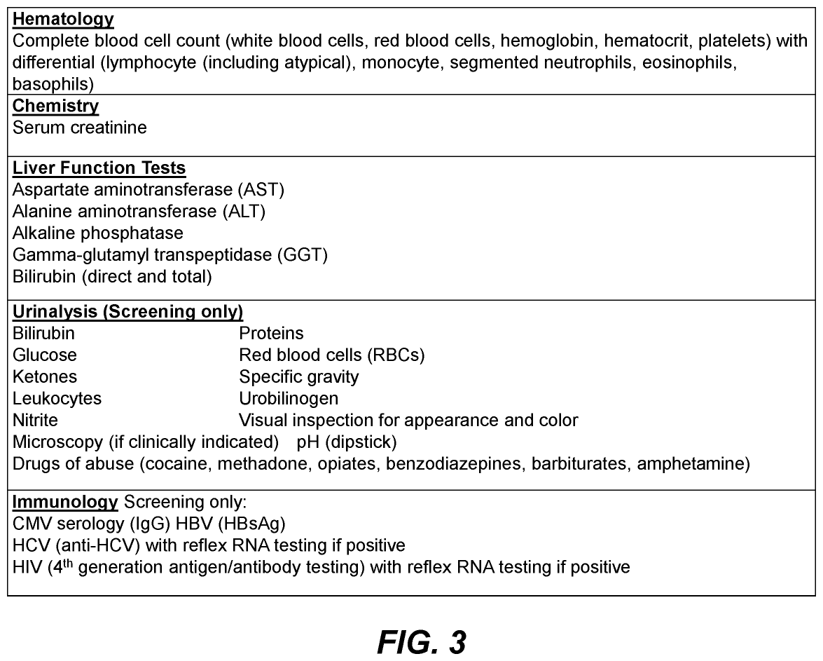

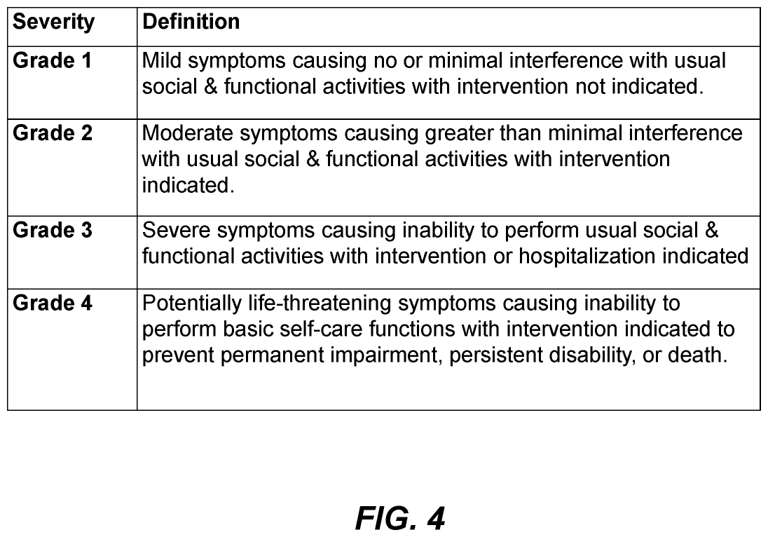

The Schedule of Assessments (SoA) used for clinical evaluation is shown in FIGS. 2A through 2F. FIG. 3 shows a list of laboratory assessments used and FIG. 4 shows grading of adverse event (AE) severity during clinical evaluation of the HCMV-based HIV vaccine.

### Medical History

A complete medical history will be collected on all subjects during screening, and will be updated as needed prior to dosing and throughout the study. A complete medical history includes details regarding medication history, illnesses and allergies, date(s) of onset, and whether the condition(s) are currently ongoing.

### Exploratory Analyses Samples

Blood samples will be collected for exploratory analyses, including but not limited to characterization of immune responses directed against HIV Gag and CMV, induction of VISP and identification of a transcriptomic signature in the peripheral whole blood elicited in response to the vaccine.

### Commercial HIV Diagnostic Testing and VISP Assessments

HIV testing using a 4^{th} generation commercial diagnostic test will be performed at screening and throughout the study. The HIV test at screening will be used to determine eligibility. The 4^{th} generation HIV diagnostic test will be used at multiple time points during the study to assess for newly acquired HIV infection. The acquisition of a true HIV infection after administration of the first dose of IP will be captured as an adverse event and communicated to the subject upon confirmation of infection. A positive HIV test due to VISP will not be captured as an adverse event.

In addition, a serum sample will be collected at Week 36 for a comprehensive assessment of VISP using multiple commercial testing modalities.

### Physical Examination

A full physical examination will be conducted at Screening, Day 1, and Day 57 visits. This includes general appearance, head/neck, chest/respiratory, heart/cardiovascular, gastrointestinal/liver/spleen, extremities, skin, and neurological assessments, as well as assessment of injection site and regional lymphatics. A symptom directed physical examination will be performed on all other visits according to the Schedule of Assessments and investigator discretion.

### Reactogenicity Assessment

Reactogenicity assessment examinations will be completed at site visits per the SoA. A reactogenicity telephone call will be performed at Week 6 to assess systemic signs and symptoms of reactogenicity, such as; fever, chills, headache, fatigue, malaise, nausea, vomiting, myalgias, and arthralgias. Subjects reporting systemic signs and symptoms during the reactogenicity telephone call will come into clinic for an unscheduled visit to assess AEs, complete a full physical examination, and clinical labs.

### Height and Weight

Height and body weight will be measured. Body mass index will be calculated from height and weight.

### Vital Signs

Vital sign measurements include blood pressure, pulse rate, temperature, and respiratory rate. Vital signs should be measured after the subject has rested comfortably for approximately 10 minutes. When scheduled for the same visit, the assessment of vital signs must be performed before physical examination and blood sample collection.

### Confirmation of Non-Childbearing Potential

A confirmation of post-menopausal status or documentation of surgical sterility must be confirmed for all female subjects.

### Viremia and Viral Shedding Assessments

If viral vector shedding is noted at the unblinding of Week 36, participants will continue to be monitored every 4 weeks (+/−1 week) until two consecutive negative viral detection assays are documented. If a decreasing trend is observed but never reaches the lower limit of detection, monitoring should continue until results demonstrate that a plateau has been reached in at least two consecutive sampling time points (4 weeks+/−one week apart) at which time discontinuation of shedding assessments may be considered with sponsor approval.

### Participant Diary

Subjects will perform a self-assessment of symptoms associated with reactions post each dose of IP. Subjects will record an assessment for local signs and symptoms at the injection site, as well as systemic signs and symptoms.

### Leukapheresis

Leukapheresis will be performed on all subjects between weeks 16 and 20. The procedure separates white blood cells from the blood, specifically the PBMC, which will be harvested for exploratory immunology analyses.

### Unscheduled Visits

Unscheduled visits are permitted at the discretion of the investigator as needed for safety assessment.

### Adverse Events and Serious Adverse Events

An adverse event is any untoward medical occurrence in a clinical study subject administered an investigation product, which does not necessarily have a causal relationship with the treatment. An AE can therefore be any unfavorable and/or unintended sign, symptom, or disease temporally associated with the use of an investigational product, whether or not considered related to the investigational product. AEs may also include pre- or post-treatment complications that occur as a result of protocol specified procedures, lack of efficacy, overdose, drug abuse/misuse reports, or occupational exposure. Pre-existing conditions which change in nature or severity should also be considered AEs.

An AE does not include the following: (1) Medical or surgical procedures such as surgery, endoscopy, tooth extraction and transfusion. The condition that led to the procedure may be an adverse event and must be reported; (2) pre-existing diseases, conditions, or laboratory abnormalities present or detected before the screening visit that do not worsen; situations where an untoward medical occurrence has not occurred (e.g. hospitalization for elective surgery); (3) overdose of investigational product without clinical sequelae; (4) any medical condition or clinically significant laboratory abnormality with an onset date before the consent form is signed and not related to a protocol-associated procedure; (5) laboratory abnormalities that are not associated with signs or symptoms; and (6) medical procedures.

Following initiation of investigational product, all AEs and new onset chronic diseases (NOCDs), regardless of cause or relationship, will be collected until 36 weeks after first administration of IP. During the LTFU, only AEs associated with the study procedures, NOCDs, and SAEs will be collected. All SAEs, regardless of cause or relationship, that occur after the subject first consents to participate in the study and throughout the duration of the study must be reported. All AEs, SAEs, and NOCDs should be followed until resolution or stabilization, if possible.

A serious adverse event (SAE) is any event that results in the following: (1) Death; (2) a life-threatening condition; (3) inpatient hospitalization or prolongation of existing hospitalization. AEs requiring hospitalization should be considered SAEs. In general, hospitalization signifies that the subject has been detained (usually involving at least an overnight stay) at the hospital or emergency ward for observation and/or treatment that would not have been appropriate in an outpatient setting or physician's office. When in doubt as to whether ‘hospitalization’ occurred or was necessary, the AE should be considered to be a SAE; (4) persistent or significant disability/incapacity; (5) congenital anomaly/birth defect in the offspring of a subject who received Vector 1; (6) other important events may be considered an SAE when, based upon appropriate medical judgment, may jeopardize the subject and may require medical or surgical intervention to prevent one of the outcomes listed in this definition.

As noted above, laboratory abnormalities without an associated AE (signs or symptoms) and/or which do not require medical intervention are not themselves recorded as AEs or SAEs. However, laboratory abnormalities that require medical or surgical intervention must be recorded as an AE or SAE, as circumstances dictate. A positive HIV test due to VISP will not be captured as an adverse event whereas the acquisition of a true HIV infection after administration of the first dose will be captured as an adverse event. AE severity should be graded using DAIDS AE Grading Table Corrected Version 2.1 (see FIG. 4). In addition to the table, all deaths related to an AE are to be classified as grade 5.

### Example 3: Prevention of Sustained Human Immunodeficiency Virus (HIV) Infection

Two HCMV-based HIV vaccines, Vector 2 and Vector 3, will be evaluated for safety, reactogenicity, tolerability, and immunogenicity in a Phase 1 umbrella study. The vaccines may be administered for the prevention of sustained Human Immunodeficiency Virus (HIV) infection.

### Clinical Background

Pathogens are most effectively targeted by a tailored immune response highlighting important nuances and complexities of the immune system. Preclinical studies have demonstrated that specific gene deletions and/or targeted genetic modifications to the rhesus cytomegalovirus (RhCMV) vector construct are often necessary to direct a protective pathogen-specific immune response against in vivo challenge with the relevant infectious agent. CMV modifications may also result in viral attenuation by restricting cell tropism and/or antagonizing mechanisms the virus normally uses to subvert host immune responses (see Table 2). Phase 1 studies in humans allow for determination of initial safety, shedding profile, and clinically relevant immunogenicity of any HCMV product candidate.

TABLE 2
Predicted Effects of Principal Genetic
Modifications of HCMV Vectored Vaccine

| Principal Molecule | |
|---|---|
| Feature | Predicted Effect |
| ΔUL82 | Reduced replication due to impaired capacity |
| | to overcome intrinsic host cell defenses |
| ΔUL78 | Less efficient epithelial cell entry and |
| | altered chemokine receptor expression |
| ΔUL128-130 | Impaired entry into epithelial and |
| | endothelial cells |
| | Immune programming of antigen specific |
| | MHC-E-restricted CD8+ T cells together |
| | with ΔUL146-147 |
| ΔUL146-147 | Immune programming of antigen specific |
| | MHC-E-restricted CD8+ T cells |
| | together with ΔUL128-130 |
| Intact UL146-147 | Immune programming of antigen specific |
| | HLA-1-restricted CD8+ T cells |
| ΔUL18 | Enhanced CD8+ T cell response to |
| | the foreign antigen |

Human cytomegalovirus is a ubiquitous virus that infects populations worldwide. Prevalence rates range from 50% to 99% and vary by country and socioeconomic status, with people from less well-resourced countries and those with lower socioeconomic status having higher prevalence rates (Pass RF. Cytomegalovirus. In: Knipe D M, et al., eds. Fields Virology. 4th ed. Philadelphia, PA: Lippincott Williams & Wilkins; 2676-705 (2001); Staras S A, et al., Seroprevalence of cytomegalovirus infection in the United States, 1988-1994. Clin Infect Dis. 43 (9), 1143-51 (2006)). In the US, approximately 50% of all individuals are CMV seropositive by the age of 30 and among adults, the rate of seroconversion is approximately 2% per year (Hyde T B, et al., Cytomegalovirus seroconversion rates and risk factors: implications for congenital CMV. Rev Med Virol. 20(5), 311-26 (2010); Lamarre V, et al., Seroconversion for cytomegalovirus infection in a cohort of pregnant women in Quebec, 2010-2013. Epidemiol Infect. 144(8), 1701-9 (2016)). Primary infection with CMV is generally asymptomatic although it can cause a self-limiting mononucleosis-like illness. When more severe disease does occur, it usually affects individuals with immature or impaired immune defenses, as observed in congenital infection and primary or recurrent infection of individuals immunosuppressed through genetic defects, iatrogenic medications or late-stage AIDS. Overall, natural CMV exhibits very low virulence, attributable to robust host barriers that permit infection but limit CMV disease after millions of years of co-evolution of the virus and its human host. Furthermore, co-evolution has rendered CMV highly species-specific such that it causes infection only in the human host.

Phase 1 studies with Towne and Towne-Toledo chimera strains in both CMV seropositive and seronegative individuals demonstrated overall safety with no SAEs when UL82 and UL78 genes were intact and pentamer components, UL128 or UL130 were disrupted. Furthermore, challenge studies using a more wild-type strain of CMV demonstrated no SAEs and all observed clinical symptoms and laboratory abnormalities were mild to moderate, self-limiting and did not require treatment.

Previous experience with HCMV vaccines in human clinical studies comes from efforts to develop a vaccine to prevent CMV disease in pregnant women and immunocompromised individuals reported over the past 45 years. To date, live HCMV vaccines have consisted of attenuated strains extensively passaged in tissue culture (Neff B J, et al., Clinical and laboratory studies of live cytomegalovirus vaccine Ad-169. Proc Soc Exp Biol Med. 160(1), 32-7 (1979); Plotkin S A, et al., Protective effects of Towne cytomegalovirus vaccine against low-passage cytomegalovirus administered as a challenge. J Infect Dis. 159(5), 860-5 (1989); Quinnan 1984), chimeras of attenuated-wildtype strains (Heineman T C, et al., A phase 1 study of 4 live, recombinant human cytomegalovirus Towne/Toledo chimeric vaccines. J Infect Dis. 193(10), 1350-60 (2006); Adler S P, et al., A Phase 1 Study of 4 Live, Recombinant Human Cytomegalovirus Towne/Toledo Chimera Vaccines in Cytomegalovirus-Seronegative Men. J Infect Dis. 214(9), 1341-8 (2016)) and replication deficient CMV (Adler S P, et al., V160-001 Study Group. Phase 1 Clinical Trial of a Conditionally Replication-Defective Human Cytomegalovirus (CMV) Vaccine in CMV-Seronegative Subjects. J Infect Dis. 220(3), 411-419 (2019)). Early clinical efficacy studies to evaluate the attenuated Towne vaccine candidate also utilized the low passage Toledo strain as a surrogate for wildtype CMV challenge. Cumulatively, these prior clinical studies provide a broad experience of safety across a range of attenuated CMV strains and across diverse populations including CMV seropositives, CMV seronegatives (men, women, & male children) and renal transplant recipients (Plotkin S A, et al., Towne-vaccine-induced prevention of cytomegalovirus disease after renal transplants. Lancet. 1(8376), 528-30 (1984)). Similar to Vector 1 and the Vector 2 and Vector 3 vaccine candidates, the Towne strain and Towne-Toledo chimeras all contain a disruption in one or more genes that make up the pentameric complex which is required for viral entry into epithelial and endothelial cells thereby restricting cell tropism (Adler S P, et al., A Phase 1 Study of 4 Live, Recombinant Human Cytomegalovirus Towne/Toledo Chimera Vaccines in Cytomegalovirus-Seronegative Men. J Infect Dis. 214(9), 1341-8 (2016); Suárez, N. M., et al., Genomic analysis of chimeric human cytomegalovirus vaccine candidates derived from strains Towne and Toledo. Virus Genes 53, 650-655 (2017)). However, the four Towne-Toledo chimeras retained the UL82 and UL78 genes that are absent from CMV backbone vectors in which one or the other of their promoters is used to drive HIV antigen expression (see Table 2 and Table 3). Studies evaluating Towne and Towne-Toldeo chimera strains in both CMV seropositive and seronegative individuals demonstrated overall safety with no SAEs observed, none to mild/moderate clinical symptoms and rare, mild to moderate laboratory abnormalities. These studies indicate that HCMV attenuation was observed when UL82 and UL78 genes were intact and pentamer components, UL128 or UL130 were disrupted. None of the four chimeric viruses was recovered from blood, urine or saliva (Adler S P, et al., A Phase 1 Study of 4 Live, Recombinant Human Cytomegalovirus Towne/Toledo Chimera Vaccines in Cytomegalovirus-Seronegative Men. J Infect Dis. 214(9), 1341-8 (2016)). The Toledo challenge strain was not sequenced until it had undergone further passages in tissue culture which may have resulted in additional mutations not present in the challenge virus used. While it caused symptomatic infection in seropositive and seronegative recipients, there were no SAEs and all observed clinical symptoms and laboratory abnormalities resulting from challenge with this more wild-type Toledo strain were mild to moderate, self-limiting and did not require treatment. Taken together, these data from prior HCMV candidate vaccines and the Toledo strain challenge support the safe use of HCMV vectors in human vaccine studies.

### HCMV Vectors

HCMV vaccines Vector 2 and Vector 3 contain recombinant HCMV vectors derived from the clinical isolate TR-HCMV genetically modified to generate the transgene CMV vector backbone. The CMV vector backbone has been engineered to have the unique ability for both antigen delivery and immune programming (ADIP) thereby acting as a vehicle to deliver immunogens relevant to the therapeutic and/or prophylactic indication. The distinct molecular attributes of the Vector 2 and Vector 3 are described in Table 3.

TABLE 3
Attributes of Vector 2 and Vector 3

| Principal Genetic | | |
|---|---|---|
| Modifications | Vector 3 | Vector 2 |
| Transgene | HIV M conserved | HIV M conserved |
| | gag/nef/pol fusion | gag/nef/pol fusion |
| | episensus 1 | episensus 1 |
| Vector Backbone | CMV vector backbone | CMV vector backbone |
| Pentameric Complex | ΔUL128-130 | ΔUL128-130 |
| Components | | |
| Transgene Location | ΔUL78 | ΔUL82 |
| Potential Immune | ΔUL146-147 | ΔUL146-147 |
| Programming | ΔUL18 | ΔUL18 |
| (in combination | | |
| with ΔUL128-130) | | |

### Study Design

This Phase 1 umbrella study will be a blinded, multiple ascending dose study in which the two candidates, Vector 2 and Vector 3, will be evaluated individually in both CMV seropositive and CMV seronegative cohorts of participants. The starting dose, dose range and dose regimen of Vector 2 and Vector 3 in the Phase 1 umbrella study are supported by the existing nonclinical data generated from the HCMV vectored vaccine platform, in addition to Vector 2 and Vector 3 specific nonclinical studies and the available clinical safety and immunogenicity data collected from the ongoing Phase 1 study of another HCMV HIV vaccine being evaluated in CMV seropositive participants.

### Dosage Form, Route of Administration and Dosing Regimen

Vector 2 and Vector 3 will be provided in single use glass vials in histidine trehalose (HT) buffer (20 mM L Histidine, 10% w/v Trehalose, pH 7.2). The contents of the vial will be diluted to deliver the designated amount and prepared to be administered as a ≤1 mL subcutaneous (SC) injection in the deltoid area of the upper arm. The vaccine dosing regimen will consist of two doses, a prime and a boost dose.

### Study Populations

The study with Vector 2 and Vector 3 will be conducted in CMV seropositive adults to include males as well as females of non-childbearing potential with key inclusion/exclusion criteria designed to minimize any potential risk to participants and close contacts.

In addition to CMV seropositive individuals, study arms to evaluate the safety and immunogenicity of Vector 2 and Vector 3 in CMV seronegative individuals will be included in the study. One of the goals of including seronegative individuals in this study is to facilitate the selection of a single dose that is both safe and immunogenic across all individuals regardless of underlying CMV status. Overall, natural CMV exhibits very low virulence, attributable to robust host barriers that permit infection but limit CMV disease after millions of years of co-evolution of the virus and its human host. Primary CMV infection in healthy individuals is largely asymptomatic although it can also cause a self-limiting mononucleosis-like illness. Prior CMV vaccines have been safely studied in CMV seronegative participants that have included men, women, male children and renal transplant recipients. The starting dose in CMV seronegatives will be 5×10⁴ ffu, which is 20-fold less than the 1×10⁶ ffu dose of Vector 1, and will incorporate a dose escalation plan gated on safety monitoring as outlined further below.

The goal for study participant enrollment is to ensure the safety of participants and close contacts, specifically to prevent the potential for CMV disease in high risk individuals (pregnant women and immunocompromised individuals) through incorporation of rigorous study eligibility criteria. Eligibility criteria has been modified to continue ensuring participant safety while also refining eligibility criteria where risk is low. CMV transmission occurs via direct contact with infected body fluids, receipt of infected blood/tissue or through vertical transmission from mother to fetus. Direct transmission through body fluids requires close contact as defined by intimate exposure, not just close proximity. Health care workers (HCW) practicing universal precautions pose no risk of transmitting CMV to patients under standard patient interactions. While daycare providers are at higher risk of acquiring CMV from children they do not pose a transmission risk to children in their care (Adler S P, Cytomegalovirus and Child Day Care. NEJM 321, 1290-1296 (1989)). The risk of transmission in the daycare setting is from child->child as well as child->provider. Given the virology and epidemiology of CMV transmission, HCW and childcare providers are included as eligible participants in HCMV vaccine studies as they pose no additional risk of transmission to others. Expanding on this, participants who have “intimate contact” with pregnant women or immuncompromised individuals will be excluded as these conditions can result in CMV transmission between adults.

CMV is commonly acquired in childhood and causes a largely asymptomatic or rarely, a mild infection. Outside of birth and breastfeeding, CMV acquisition in children most commonly occurs from other young children, particularly in daycare/preschool settings. The seroprevalence of CMV IgG in children aged 1-5 was 28.2% in 2017/2018, up from 20.7% in 2011/2012 (Petersen M R, et al., Changes in Cytomegalovirus Seroprevalance Among U.S. Children Aged 1-5 Years: The National Health and Nutrition Examination Surveys. Clin Infect Dis. 72(9), e408-e411 (2021)). CMV transmission between an adult and child can theoretically occur through activities that promote sharing of saliva (ex. mouth kissing, sharing food utensils or drinks, pre-mastication of food for infants). However, this mode of transmission is not considered to be a significant source of primary infection in children but rather might be a mode of transmission from a child to an adult. Given that children are naturally exposed to CMV early in life and do not represent a high risk group upon infection, children under the age of 6 may be included in the study.

### Dose Escalation Schema in CMV Seronegative Participants

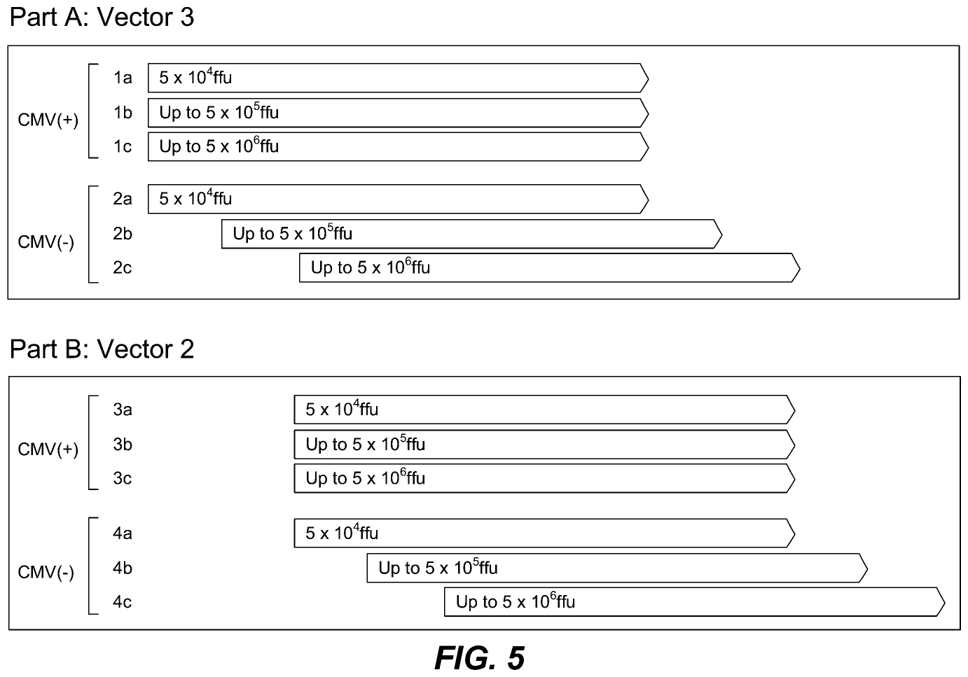

Evaluation of Vector 2 and Vector 3 in CMV seronegative participants will follow a multiple ascending dose escalation, starting with a 5×10⁴ ffu dose (FIG. 5). To protect the safety of volunteers participating in the clinical study, the SRC will perform safety data review prior to initiation of dosing a new cohort in accordance with the SRC Charter. Stepwise progression to a higher dose level will be initiated after all available safety data through 8-weeks (including adverse events, vital signs, clinical laboratory results and CMV viral detection assay results) from at least the first 6 participants in the most recent cohort and all previously dosed participants in the preceding lower dose level(s) have been evaluated by the SRC. An 8-week interval was selected based on prior vaccine studies of attenuated HCMV vaccines, which included CMV seronegative participants, and the fact that new immunologic and systemic effects of the vaccine are not anticipated to occur after 8 weeks (Adler S P, et al., A Phase 1 Study of 4 Live, Recombinant Human Cytomegalovirus Towne/Toledo Chimera Vaccines in Cytomegalovirus-Seronegative Men. J Infect Dis. 214(9), 1341-8(2016); Heineman T C, et al., A phase 1 study of 4 live, recombinant human cytomegalovirus Towne/Toledo chimeric vaccines. J Infect Dis. 193(10), 1350-60 (2006); Quinnan G V Jr, et al., Comparative virulence and immunogenicity of the Towne strain and a nonattenuated strain of cytomegalovirus. Ann Intern Med. 101(4), 478-83 (1984)). In addition to the required safety data from the CMV seronegative participants, the SRC will also have cumulative safety data from CMV seropositive participants who have received an expanded range of doses (5×10⁴ ffu, 5×10⁵ ffu or 5×10⁶ ffu) of Vector 2 or Vector 3 as described below (FIG. 5). The CMV seropositive participants will be enrolled concurrently from the time the lowest starting dose is given to the CMV seronegative subjects and will provide additional safety information for the SRC to consider. Based on this review of safety data, a recommendation will be made as to whether or not to initiate the next cohort.

With respect to the second dose (boost), the site investigator will review each participant's data record and if no individual stopping rules are met, the participant will receive a second subcutaneous dose on Day 84 (week 12). The second dose will be the same product and dosage level received during their first dose.

The SRC will provide ongoing study oversight regarding potential safety issues or in the event a study stopping rule is reached. Cohort stopping rules for will include 1)≥2 participants experience the same treatment-related Grade 3 or higher adverse event, 2) any participant experiences a treatment-related SAE or 3) any subject experiences documented end-organ disease attributable to the HCMV vector other than mild, self-limited mononucleosis-like syndrome, as determined by signs, symptoms, laboratory findings and detection of vaccine vector in relevant site(s). Vector 2 and Vector 3 also retain susceptibility to ganciclovir.

### Dose Schema to Evaluate Doses in Parallel in CMV Seropositive Participants

CMV seropositive participants will be enrolled and randomized separately to receive Vector 2 or Vector 3 and the safety and immunogenicity of doses of 5×10⁴ ffu, 5×10⁵ ffu or 5×10⁶ ffu will be evaluated (FIG. 5). All 3 dose cohorts will be initiated concurrently in CMV seropositive participants. The availability of this expanded dose range of safety data also supports dose escalation of the CMV seronegative cohorts.

### Primary Study Endpoints: Safety, Reactogenicity and Tolerability

Assessments for the two Vector 2 and Vector 3 HCMV vaccine candidates will include clinical monitoring for 1) vaccine reactogenicity, 2) signs and symptoms of CMV disease and 3) virologic detection of the HCMV vector. Assessment of vaccine reactogenicity will include both local and systemic parameters and will be conducted via in person clinical evaluations and through participant reported diaries. Evaluation of possible CMV-related disease will be conducted through clinical laboratory tests, physical exams and symptom directed review. Taken together, these evaluations will allow for the detection of both symptomatic and asymptomatic signs/symptoms of CMV-mediated disease in study participants.

The capacity for either of the HCMV candidate vectors to shed will be evaluated through PCR-based virologic detection assays. Participants will provide saliva and urine specimens at study visits to evaluate for vector shedding as well as blood samples to assess for virus in the circulation. The PCR-based tests will permit differentiation between wildtype CMV and the Vector 2 and Vector 3 vaccine vectors. Importantly, detection of HCMV nucleic acid by PCR assay does not indicate the presence of intact nor infectious virus; however it is the most sensitive and conservative approach to assess for vector or wild type CMV shedding. Further, the capacity for HCMV vector shedding does not equate to transmissibility nor ability to cause disease in a contact. Evaluation for vector transmission will be considered in future studies if significant vaccine vector shedding is detected.

### Secondary and Exploratory Endpoints: Immune Response Characterizations

Many pathogens that evade natural immune responses may be susceptible to control by the high frequencies of antigen specific T cells expected to be elicited by vaccination with the relevant HCMV vector. Regardless of the foreign antigen being expressed, HCMV vectors have the potential to generate robust effector differentiated memory CD4+ T cells as well as CD8+ T cells, capable of recognizing HLA-E, HLA class 1 or HLA class 2-mediated antigen presentation. The immune response is anticipated to encompass a T cell repertoire covering a breadth of epitopes not observed with traditional live-attenuated or protein/adjuvant vaccines and these antigen-specific T cells are expected to be maintained both in the circulation and in tissues (Hansen S G, et al., A live-attenuated RhCMV/SIV vaccine shows long-term efficacy against heterologous SIV challenge. Sci Transl Med. 11(501), eaaw2607 (2019)).

The secondary endpoints aim to characterize the immune response induced by Vector 2 and Vector 3 as measured by T cell and antibody responses to vaccine-derived HIV-1 M conserved gag/nef/pol fusion episensus 1 (containing epitopes from Gag, Pol and Nef). The magnitude, function and phenotypic profile of M conserved gag/nef/pol fusion episensus 1CD4 and CD8 T cell responses will be assessed by intracellular cytokine staining (ICS) and flow cytometry. Serologic titer of M conserved gag/nef/pol fusion episensus 1 epitope-specific binding antibodies will also be evaluated.

Exploratory endpoints are intended to more deeply characterize the nature of the immune response generated and will include evaluation of the breadth of T cell epitopes, HLA epitope restriction, expanded functional and phenotypic profiles and the transcriptomic profile in peripheral whole blood to identify any potential immune signatures of vaccine take. Additionally, the presence, distribution and magnitude of CD4 and CD8 T cells may be evaluated through mucosal biopsy and lymph node aspirate to understand how antigen-specific T cells traffic in the tissues at sites of primary infection and peripheral immune tissues to amplify the immune response.

### Chemistry, Manufacturing and Controls Background

### Vector Backbone

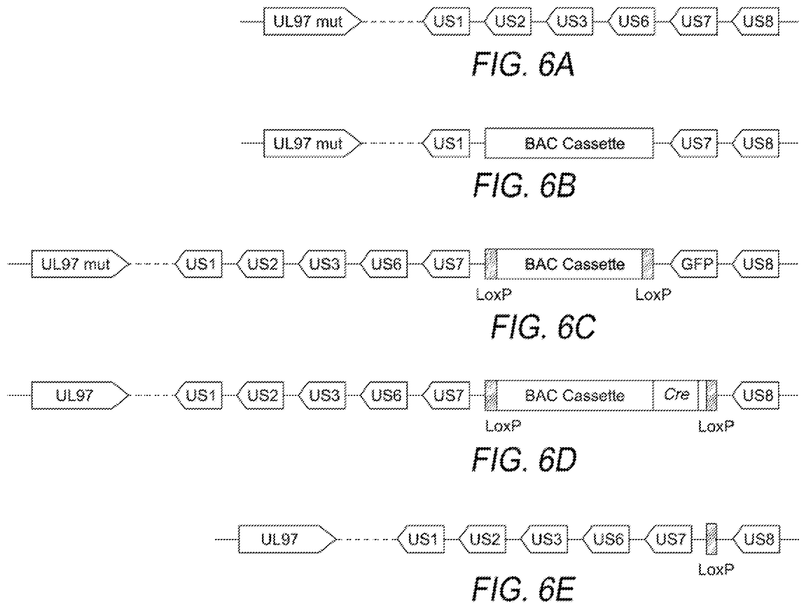

The HCMV strain TR was selected as the vector backbone since its genomic organization represents a typical clinical isolate (Murphy E, et al., Coding potential of laboratory and clinical strains of human cytomegalovirus. Proc Natl Acad Sci USA. 100(25), 14976-81 (2003)). The HCMV TR genome was cloned into a bacterial artificial chromosome (BAC) to allow for modification in E. coli (FIG. 6A). As a result of this process, the genomic region US2-US6 was deleted (FIG. 6B) (Murphy 2003). To restore the region deleted during BAC cloning, the US2-US7 genes from HCMV strain AD169 were inserted in the HCMV TR-BAC along with the addition of GFP and LoxP sites flanking the BAC cassette (FIG. 6C) (Lauron E J, et al., Human cytomegalovirus infection of Langerhans-type dendritic cells does not require the presence of the gH/gL/UL128-131A complex and is blocked after nuclear deposition of viral genomes in immature cells. J Virology 88(1), 403-16 (2014)). Since the HCMV TR strain was originally isolated from a patient with late-stage AIDS and was initially ganciclovir-resistant due to a mutation in the kinase gene UL97 (Smith I L, et al., High-level resistance of cytomegalovirus to ganciclovir is associated with alterations in both the UL97 and DNA polymerase genes. J Infect Dis. 176(1), 69-77 (1997)), sensitivity to the antiviral effects of ganciclovir was restored by replacing the mutated TR UL97 with intact UL97 from HCMV AD169 (FIG. 6D) (Bradley A J, et al., High-throughput sequence analysis of variants of human cytomegalovirus strains Towne and AD169. J Gen Vir. 90(10), 2375-80 (2009)). In addition, the GFP gene was removed and Cre recombinase under control of the SV40 early promoter was added to the BAC cassette to render it self-excising in mammalian cells (FIG. 6D) (Caposio P, et al., Characterization of a live-attenuated HCMV-based vaccine platform. Sci Rep 9, 19236 (2019)). The resulting vector is the CMV vector backbone (FIG. 6E).

### Vector Construction and Characterization

The CMV vector backbone BAC has been modified to generate the final HCMV-HIV vaccine vectors, Vector 2 and Vector 3. The modifications were accomplished by sequential recombination steps of the CMV vector backbone BAC in E. coli. Standard BAC recombineering using galactokinase/kanamycin (galK/Kan) recombination (Warming S, et al., Simple and highly efficient BAC recombineering using galK selection. Nucleic Acids Res. 33(4), e36 (2005)) was performed to introduce either deletions or transgene replacements. The final BAC vectors have been sequenced by next generation sequencing (NGS) to confirm the intended modifications compared with the CMV vector backbone.

### Cell Substrate

Vector 2 and Vector 3 are manufactured in a human diploid fibroblast cell line, MRC-5. A Working Cell Bank (WCB) of MRC-5 has been manufactured under cGMP and tested to International Council for Harmonisation (ICH)/United States Food and Drug Administration guidelines. Recombinant viruses are rescued from Working Cell Bank (WCB) cells transfected with a recombinant viral genome cloned as a BAC in E coli.

### Manufacturing of Vector 2 and Vector 3

Vector 2/Vector 3 Drug Product will be manufactured using a Master Seed Virus (MVS) for each product and the Research Seed Stock (RSS) is the starting material for the MVS. To begin RSS production, the Vector BAC DNA is propagated in E. coli from a glycerol stock generated during the final recombination steps of the BAC construction described above. The BAC DNA is isolated and purified from the E. coli using standard recombinant DNA protocols. Characterization of the final RSS product will include quantitation, restriction digest for integrity and NGS for identity (see Table 4).

TABLE 4
RSS Generation Steps and Testing
Appendix A.

| Steps | Final Testing |
|---|---|
| Vector BAC | DNA concentration for quantitation, |
| DNA production | restriction digest for integrity, |
| | and NGS for identity |
| ↓ | |
| Virus reconstitution | LA-IFA for infectious titer, |
| in MRC-5 WCB | antigen immunoblot for function |
| P00161 +/− | and NGS for identity |
| UL82 mRNA -P0 | |
| ↓ | |
| Virus amplification in | LA-IFA for infectious titer, antigen |
| MRC-5 cells in | immunoblot for function, NGS for identity, |
| Hyperstack vessels | safety assays including mycoplasma, |
| to generate RSS - P1 | bovine viruses, and sterility |

LA-IFA: late antigen immunofluorescence assay

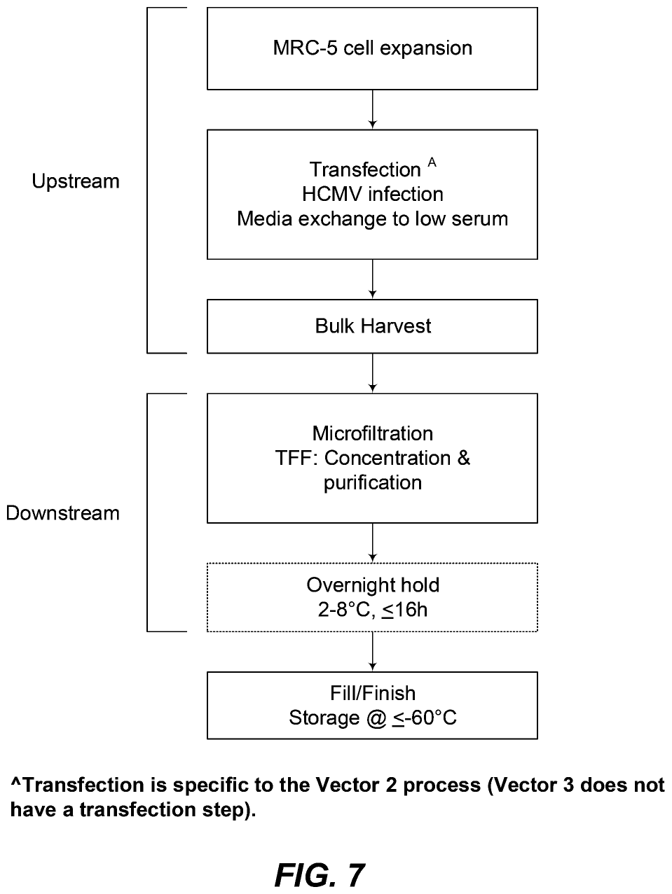

The Master Virus Seed (MVS) and Clinical Trial Material (CTM) manufacturing processes for Vector 2 and Vector 3 are presented in FIG. 7. The manufacturing process for each MVS (i.e., corresponding to each product) is identical to the process utilized for CTM production, with the exception that the MVS is seeded with RSS.

The cGMP manufacturing process consists of reconstitution and expansion of virus in WCB cells to prepare a MVS. The MVS is further expanded by infecting additional WCB cells to manufacture the CTM for each vaccine product. The harvest obtained from infected WCB production cultures is clarified by microfiltration. The clarified harvest is concentrated and purified by double diafiltration into the final formulation buffer to prepare the intermediate bulk (i.e., bulk material prior to Fill/Finish). Following a short-term hold step, the intermediate bulk is then filled in single use vials to produce Drug Product (DP, also referred to as the CTM).

For Vector 2 and Vector 3 MVS and CTM manufacturing, intermediate bulk is held in bags (filled at a 30% volume to bag size ratio) and stored at 2-8° C. for up to 16 hrs prior to further processing. Prior to the fill/finish vialing step, the bulk bag (containing intermediate bulk) is brought to room temperature (RT) for ≥2 hours with constant mixing by rocking and then vialed using a fully automated fill/finish setup.

Both MVS and CTM are vialed at 0.7 mL (extractable) fill volume. The total fill finish process, including QC inspection, is expected to take <12 hours. At the completion of fill/finish, the vials are stored at ≤−60° C.

### Intermediate Hold-Time

For Vector 2 and Vector 3 manufacturing (to support Phase 1 clinical studies), HT buffer (Histidine and Trehalose) is used as the final formulation. This formulation provides sufficient stability to the intermediate bulk to support extended holds. Therefore, a hold step was implemented in between the downstream process (DSP) and Fill/Finish to provide adequate flexibility within the intended GMP manufacturing process. The aforementioned improvements relative to an earlier HCMV manufacturing process are summarized in Table 5.

TABLE 5
Downstream process comparison

| Parameters/Attributes | Earlier HCMV Vector | Vector 2/Vector 3 |
|---|---|---|
| Formulation | TNS Buffer | HT Buffer |
| | 50 mM Tris, | 20 mM L-Histidine, |
| | 150 mM NaCl, | 10% Trehalose, |
| | 10% Sucrose, pH 8.0 | pH 7.2 |
| Hold Step | No hold | Up to 16 hours |

### Hold Time Studies

Various studies have been performed to date that support the stability of HCMV intermediate bulk under various hold conditions (e.g., temperature and duration). These studies utilized infectious titer as the primary stability indicating test attribute and are further described below.

### Hold-Time in Bioprocessing Bags

A study was performed to assess the effect of hold-time of up to 72 hrs in bioprocessing bags on infectious titer. The study utilized two bag types, CX5-14 Labtainer™ PE (polyethylene) and Flexboy® EVA (ethylene vinyl acetate), tested at multiple fill volumes and hold durations.

All bags were filled with representative intermediate bulk at 10% and 75% volume to bag size ratio (to bracket the 30% fill for GMP production) and sampled after lying flat for a 72-hour hold at 2-8° C. The samples were analyzed for infectious titer by a late antigen immunofluorescence assay (LA IFA) to determine titer loss relative to the T=0 titer corresponding to study start.

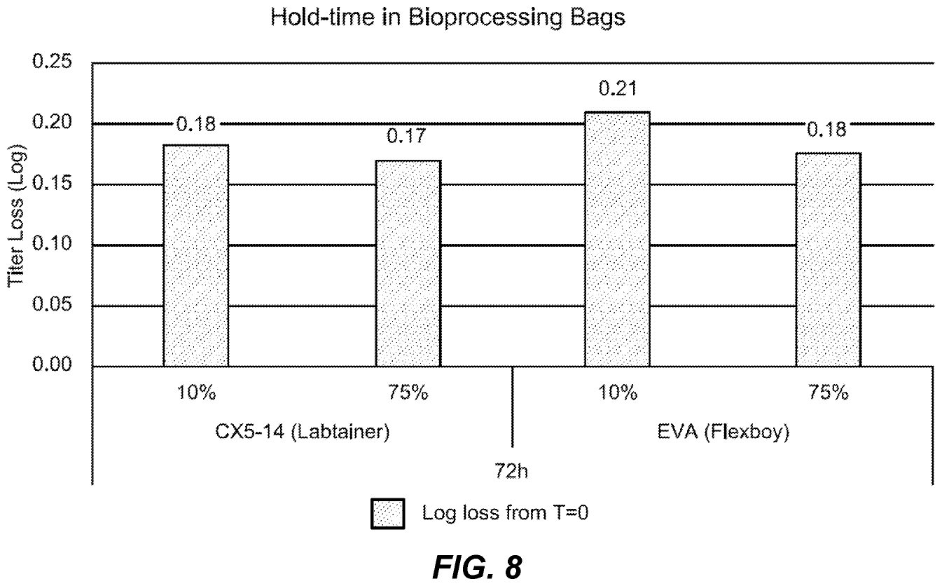

As presented in FIG. 8, results are comparable between the PE (CX5-14 Labtainer™) bag used in Vector 2 and Vector 3 MVS and CTM manufacturing, and EVA (Flexboy®) material; a hold-time of 72 hours at 2-8° C. results in a maximum titer loss of 0.21 log in the infectious titer across all conditions.

### Cumulative Hold-Time Study

To simulate the GMP manufacturing process where intermediate bulk is held in bioprocessing bags overnight and then filled in vials at room temperature, a cumulative hold-time study was performed. Representative intermediate bulk formulated in HT buffer was held in a FlexBoy® bag, filled to 30% of capacity, overnight (“O/N”) at 2-8° C. for 16 hours. Following the overnight hold, intermediate bulk was held out to 72 hours at RT, after which it was filled at 0.7 mL in vials and held for an additional 48 hours at RT to mimic a worst-case scenario for RT holds.

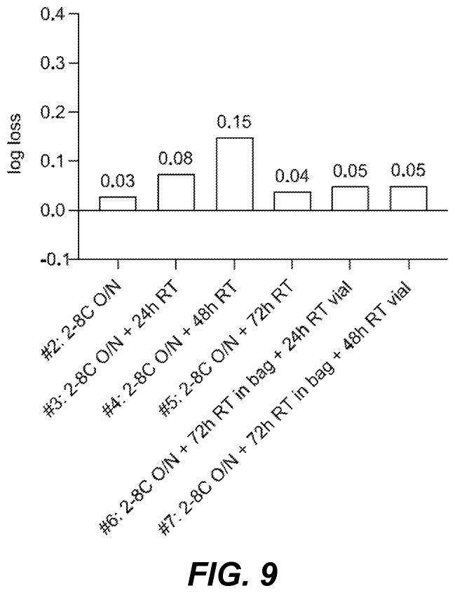

As presented in FIG. 9, all conditions maintained a titer loss of less than 0.2 log, which is within the variability of the LA-IFA assay. While titer loss is slightly lower than results obtained from the hold-time study presented in the previous section (“Hold-time in Bioprocessing Bags”), all results are within 0.5 log of T=0 and therefore not considered analytically significant based on current process understanding and analytical method capability. The results from both hold-time studies demonstrates that product titer is not impacted by “worst-case” hold conditions that exceed the maximum allowable hold durations for GMP production.

In addition to analysis of infectious titer, the conditions shown in FIG. 8 were tested for pH and visual appearance prior to freezing. The pH attribute remained within specification (pH 7.2±0.5, noting that the initial intermediate bulk pH was 7.1) and contents of all samples were clear in appearance satisfying the criteria of “clear to opalescent; white particulates may be present”. All pH and appearance results are presented in Table 6; the table also presents the titer results (from FIG. 8) in tabular form.

TABLE 6
Titers, pH, and Appearance of Intermediate Bulk in a Cumulative Hold-time Study

| | | | | | Log diff | | |
|---|---|---|---|---|---|---|---|
| | | | Storage | Timepoint | from | | |
| Description | | Vessel | condition | (hr) | T = 0 | pH | Appearance |
| Acceptance criteria | | | | | NA¹ | 6.7-7.7 | Clear to opalescent; |
| | | | | | | | white particulates |
| | | | | | | | may be present |
| Bottle | | bottle | NA | T = 0 | 0.000 | 7.1 | NA |
| thawed | | | | (F/T) | | | |
| 16 | h at | bag | 2-8° C. | T = 16 | −0.03 | 7.1 | Meets acceptance |
| | | | | | | | criteria |
| 24 | h at RT | bag | RT | T = 24 | −0.08 | 7.1 | Meets acceptance |
| | | | | | | | criteria |
| 48 | h at RT | bag | RT | T = 48 | −0.15 | 7.0 | Meets acceptance |
| | | | | | | | criteria |
| 72 | h at RT | bag | RT | T = 72 | −0.04 | 7.1 | Meets acceptance |
| | | | | | | | criteria |
| 96 | h at RT | vial | RT | T = 96 | −0.05 | 7.1 | Meets acceptance |
| | | | | | | | criteria |
| 120 | h at RT | vial | RT | T = 120 | −0.05 | 7.1 | Meets acceptance |
| | | | | | | | criteria |

F/T: freeze/thaw;
NA: not applicable;
O/N: over night;
RT: room temperature
¹Considering the anticipated high titers for Vector 2 and Vector 3 and the analytical variability for the LA IFA method (i.e., ±0.2 log), a log loss of less than 0.5 is unlikely to impact product quality or introduce risk to clinical dose preparation. Therefore, an acceptance criterion was not applied.

### Low Residual BAC DNA in the Clinical Trial Material

As outlined in “Chemistry, Manufacturing and Controls Background”, production of the viral seed stock begins with a bacterial artificial chromosome (BAC) grown in E. coli that is purified and then transfected into MRC-5 cells for virus reconstitution. This BAC encodes the entirety of the Vector 2/Vector 3 viral genomes in addition to a self-excising cassette. This cassette contains genes for the maintenance of the BAC in E. coli in addition to the Cre recombinase gene under control of an eukaryotic promotor. Expression of the Cre recombinase in MRC-5 cells is used to excise the BAC cassette which is located between the two LoxP sites, from the viral genome (FIGS. 6A-6E). Residual BAC DNA may be present as self-excision by the Cre recombinase is not 100% efficient.

Low levels of residual BAC DNA have been detected in Vector 2/Vector 3 MVS. Characterization testing was performed using a qPCR assay to detect a small region of the chloramphenicol gene in the BAC as described in the Vector 1 IND submission. Using this qPCR assay to the chloramphenicol gene, the copies of BAC DNA present in Vector 2/Vector 3 are shown in Table 7 in addition to the number of total viral genomes determined by qPCR assay to the UL79 viral gene. To estimate the amount of BAC DNA per dose, the full-length BAC DNA (8,222 bp) molecular weight was used to convert the copies/mL from the chloramphenicol qPCR assay to the ng/dose reflecting the maximum amount of residual full-length BAC DNA.

TABLE 7
Characterization Data for Residual BAC DNA

| | | | Total | Percent of | BAC |
|---|---|---|---|---|---|
| | | BAC | Viral | BAC DNA | DNA |
| | | DNA | Genomes/ | to Viral | ng/ |
| Vector | Material | copies/mL | mL | Genomes | dose^{a} |
| Vector 2 | MVS | 91,688 | 2.9E+09 | 0.0032% | 0.00038 |
| Vector 3 | MVS | 134,026 | 3.6E+09 | 0.0037% | 0.00056 |

^{a}The ng per dose were calculated based on a final titer of 1e+07 FFU/mL in the drug product and a clinical dose of 5e+06 FFU

To determine if full-length BAC DNA was present, junctional PCR primers were developed that amplified across the viral/BAC junctions at both the 5′ (US7) and 3′ (US8) regions (FIGS. 6A-6E). All materials tested resulted in positive junctional PCR reactions, showing that full-length BAC DNA is present in some percentage of viral genomes. While the actual percentage of full-length BAC present in viral genomes is unknown, worst case levels are extremely low, as shown in Table 7.

The residual BAC DNA data may be considered within the context of the FDA/WHO guidelines (and corresponding limits) on residual host cell DNA. Based on this guidance, the amount of host cell DNA should be less than 10 ng/dose and less than 200 bp in length. Although the size of the BAC DNA fragments may be much larger than 200 bp, the estimated amount of BAC DNA per Vector 2/Vector 3 dose is well below this limit. In order to assess the risk from residual BAC DNA in terms of oncogenicity, infectivity, and immunogenicity, the genes in the BAC DNA are summarized below:
Bacterial genes (sopA, sopB, sopC, repE, and resD) in addition to a chloramphenicol resistance gene are present and are under the control of bacterial promotors. These genes allow for the maintenance of the BAC while it is being produced in E. coli during manufacturing.
A Cre recombinase gene under control of the SV40 promoter whose expression drives the self-excision of the BAC cassette between two LoxP sites, leaving behind a single LoxP site between HCMV genes US7 and US8.

All genes under the control of bacterial promotors would not have the ability to be transcribed and translated in a human cell and pose no risk to patient safety. The Cre recombinase gene could potentially be expressed in a human cell using the SV40 eukaryotic promotor and would continue to remove the BAC DNA between residual LoxP sites from the vector genome.

Unlike host cell DNA that may contain oncogenic DNA sequences and/or potentially infectious viral DNA sequences from latent viruses, the BAC DNA contains no known oncogenes and/or infectious DNA sequences. In terms of immunogenicity, BAC DNA has the potential to trigger intrinsic host cell defenses rather than antigen-specific responses that would be expected to be elicited by a plasmid designed to express a protein for gene therapy or vaccination.

Based on the low level of residual BAC DNA per dose and given the known characteristics of the BAC DNA, this impurity does not pose any safety risk to the participating clinical trial subjects.

### List of Abbreviations

| Term | Definition |
|---|---|
| Δ | deletion of the following genes specified, eg ΔUL82 |
| ADIP | Antigen Delivery and Immune Programming |
| BAC | bacterial artificial chromosome |
| BAL | bronchoalveolar lavage |
| BM | bone marrow |
| CCR7 | C-C chemokine receptor 7 |
| cGMP | current good manufacturing practice |
| CMC | chemistry manufacturing and controls |
| CMV | cytomegalovirus |
| CPE | cytopathic effect |
| CS-5 | Cellstack-5 layer |
| CTM | clinical trial material |
| DAXX | death domain associated protein or death associated |
| | protein 6 |
| DNA | deoxyribonucleic acid |
| DP | drug product |
| dpi | days post infection |
| dpv | days post vaccination |
| EC₅₀ | half maximal effective concentration |
| EDL | early development laboratory |
| FIH | first-in-human |
| FBS | fetal bovine serum |
| ffu | focus-forming units |
| gag | gene encoding group-specific antigen of HIV or SIV |
| Gag | group-specific antigen of HIV or SIV |
| gB | glycoprotein B |
| GLP | good laboratory practice |
| HCMV | human cytomegalovirus |
| HCW | Health care worker |
| HF10 | HYPERFlask-10 layers |
| HIV | human immunodeficiency virus |
| HLA | human leukocyte antigen |
| HS12 | HYPERStack - 12 layers |
| HS36 | HYPERStack - 36 layers |
| ICS | Intracellular cytokine staining |
| IE | immediate early immunofluorescence assay |
| IND | investigational new drug |
| LLOD | lower limit of detection |
| MHC | major histocompatibility complex |
| MOI | multiplicities of infection |
| mRNA | messenger ribonucleic acid |
| MVS | master virus seed |
| NGS | next generation sequencing |
| NHPs | nonhuman primates |
| NZW | New Zealand White rabbit |
| OHSU | Oregon Health & Science University |
| OR | Oregon |
| PBMC(s) | peripheral blood mononuclear cell(s) |
| PCR | polymerase chain reaction |
| PHSA | Public Health Service Act |
| POC | proof-of-concept |
| pp71 | phosphoprotein 71 |
| pol | SIV polymerase gene |
| qPCR | quantitative polymerase chain reaction |
| RhCMV | rhesus cytomegalovirus |
| RM | rhesus macaque |
| RNA | ribonucleic acid |
| RSS | research seed stock |
| SC | subcutaneous |
| SAE | safety adverse event |
| SEM | standard error of the mean |
| SIV | simian immunodeficiency virus |
| siRNA | small interfering ribonucleic acid |
| SIV | simian immunodeficiency virus |
| SOC | standard of care |
| SRC | safety review committee |
| TB | tuberculosis |
| CMV vector | recombinant human cytomegalovirus vector containing |
| backbone | intact UL82, UL97, UL128-130 and UL146-147 |
| WBC | white blood cell count |
| WCB | working cell bank |
| WT | wild type |

While specific embodiments have been illustrated and described, it will be readily appreciated that the various embodiments described above can be combined to provide further embodiments, and the various embodiments described above can be combined to provide further embodiments.

All of the U.S. patents, U.S. patent application publications, U.S. patent applications, foreign patents, foreign patent applications, and non-patent publications referred to in this specification and/or listed in the Application Data Sheet, including U.S. Provisional Patent Application Nos. 63/239,298 filed Aug. 31, 2021 and 63/356,386 filed Jun. 28, 2022, are incorporated herein by reference, in their entirety, unless explicitly stated otherwise. Aspects of the embodiments can be modified, if necessary to employ concepts of the various patents, applications, and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A recombinant HCMV vector comprising a TR3 backbone and a nucleic acid sequence encoding a heterologous antigen, wherein:
(i) the vector does not express UL18, UL78, UL128, UL130, UL146, or UL147, or orthologs thereof;
(ii) the vector comprises a nucleic acid sequence encoding UL82, or an ortholog thereof; and
(iii) the heterologous antigen replaces all or part of UL78 and is operably linked to the UL78 promoter;
wherein the heterologous antigen is a HIV fusion protein comprising an amino acid sequence having at least 90%, at least 91%, at least 92%, at least 93%, at least 94%, at least 95%, at least 96%, at least 97%, at least 98%, at least 99%, or 100% identity to the amino acid sequence according to SEQ ID NO:3.

2.-4. (canceled)

5. The recombinant HCMV vector of claim 1, wherein the vector does not express a UL18 protein, UL78 protein, UL128 protein, UL130 protein, UL146 protein, or UL147 protein, resulting from the presence of one or more mutations in the nucleic acid sequence encoding UL18, UL78, UL128, UL130, UL146, or UL147.

6. The recombinant HCMV vector of claim 5, wherein the mutation in the nucleic acid sequence encoding UL18, UL78, UL128, UL130, UL146, or UL 147 is a point mutation, frameshift mutation, truncation mutation, or deletion of all of the nucleic acid sequence encoding the viral protein.

7. The recombinant HCMV vector of claim 1, wherein the vector further comprises a nucleic acid sequence encoding a microRNA (miRNA) recognition element (MRE), wherein the MRE contains a target site for a miRNA expressed in endothelial cells or a miRNA expressed in myeloid cells.

8-20. (canceled)

21. A recombinant HCMV vector comprising a nucleic acid sequence having at least 90%, 91%, 92%, 93%, 94%, 95%, 96%, 97%, 98%, 99%, or 100% identity to the nucleic acid sequence according to SEQ ID NO:7 or 9.

22-35. (canceled)

36. A pharmaceutical or immunogenic composition comprising the recombinant HCMV vector of claim 1 and a pharmaceutically acceptable carrier.

37. (canceled)

38. A method of generating an immune response in a subject, comprising administering to the subject the recombinant HCMV vector of claim 1.

39-41. (canceled)

42. A method of treating or preventing HIV in a subject, comprising administering the recombinant HCMV vector of claim 1 to the subject.

43. A method of treating or preventing HIV in a subject, comprising administering the nucleic acid sequence according to SEQ ID NO: 7 or 9 or pharmaceutical composition comprising the nucleic acid sequence according to SEQ ID NO:7 or 9 to the subject.

44.-59. (canceled)

60. The method of claim 42, wherein the subject is seropositive for HCMV.

61. The method claim 42, wherein the subject is seronegative for HCMV.

62. The method of claim 42, wherein the recombinant HCMV is administered in an amount of at least 1×10³ focus-forming units (ffu), about 5×10⁴ ffu, about 5×10⁵ ffu, about 5×10⁶ ffu, about 1×10³ ffu, about 3×10⁴ ffu, or about 1×10⁶ ffu.

63.-69. (canceled)

70. The method of claim 38, wherein the heterologous antigen is or comprises a HIV antigen and the disease is HIV infection.

71.-76. (canceled)

77. A method of generating CD8+ T cells that recognize MHC-E/peptide complexes, the method comprising:
(a) administering to a first subject the recombinant HCMV vector of claim 1 in an amount effective to generate a set of CD8+ T cells that recognize MHC-E/heterologous antigen-derived peptide complexes;
(b) identifying a first CD8+ TCR from the set of CD8+ T cells, wherein the first CD8+ TCR recognizes a MHC-E/peptide complex;
(c) isolating one or more CD8+ T cells from a second subject; and
(d) transfecting the one or more CD8+ T cells isolated from the second subject with an expression vector, wherein the expression vector comprises a nucleic acid sequence encoding a second CD8+ TCR and a promoter operably linked to the nucleic acid sequence encoding the second CD8+ TCR, wherein the second CD8+ TCR comprises CDR3a and CDR3B of the first CD8+ TCR, thereby generating one or more CD8+ T cells that recognize MHC-E/peptide complexes.

78.-81. (canceled)

82. The method of claim 77, wherein the first subject is seropositive for HCMV.

83. The method of claim 77, wherein the first subject is seronegative for HCMV.

84. (canceled)

85. A CD8+ T cell generated by the method of claim 77.

86. A method of treating or preventing a disease in a subject, the method comprising administering the CD8+ T cell of claim 85 to the subject.

87.-88. (canceled)

89. The pharmaceutical composition of claim 36, wherein the pharmaceutically acceptable carrier comprises a histidine trehalose (HT) buffer.

90. The pharmaceutical composition of claim 36, wherein the pharmaceutically acceptable carrier comprises a histidine trehalose (HT) buffer comprising about 20 mM L-histidine and about 10% (w/v) trehalose.

91. The pharmaceutical composition of claim 36, wherein the pharmaceutical composition is lyophilized.

92. The pharmaceutical composition of claim 36, wherein the pharmaceutical composition is in a solution.

93. The method of claim 42, wherein the pharmaceutical composition is administered subcutaneously.

94. The method of claim 42, wherein the pharmaceutical composition is administered in two doses.

95. The method of claim 42, wherein treating or preventing comprises:
(i) eliciting a CD8+ T cell response to at least one HIV antigen;
(ii) reducing viremia and/or detectable HIV load, including reducing detectable HIV load below the limit of detection by any suitable test (e.g., polymerase chain reaction (PCR));
(iii) containing HIV replication and/or mutation such that primary HIV infection is rapidly aborted; and/or,
(iv) averting sustained infection and disease such that life-long antiviral treatment (ART) is not required.

96. The method of claim 95, wherein sustained infection and disease comprises:
(i) detection of at least 10,000 HIV copies per milliliter of blood and/or
(ii) detection of HIV in blood samples for three or more consecutive weeks.
